(19) United States
(12) Patent Application Publication
(10) Pub. No.: US 2026/0224256 A1
(43) Pub. Date: Aug. 6, 2026

(54) IMPLANTS, SURGICAL TOOLS FOR EXPANDING IMPLANTS AND FOR ORTHOPAEDIC SURGERY

(71) Applicant: AM Solutions Holding B.V., The Hague (NL)

(72) Inventors: Seyed Mohammad Ahmadi; Banafsheh Sajadi; Sanne Aarts

(73) Assignee: AM Solutions Holding B.V., The Hague (NL)

(21) Appl. No.: 19/153,113

(22) PCT Filed: Jan. 22, 2024
(86) PCT No.: PCT/NL2024/050030

§ 371 Date: Aug. 1, 2025

(30) Foreign Application Priority Data
Feb. 21, 2023 (NL) 2034197

(51) Int. Cl. A61B 17/84; A61B 17/56; A61B 17/70; A61B 17/86

(52) U.S. Cl. CPC A61B 17/844; A61B 17/7074; A61B 2017/564; A61B 2017/8655

(57) ABSTRACT

An expandable intra-osseous implant for a bone, e.g. vertebra, comprises a support structure defining a longitudinal axis. First and second sets of movable parts are mounted to the support structure. Each movable part comprises a load resisting surface for resisting a load external to the implant. In an unexpanded state of the implant they are circumferentially spaced around the longitudinal axis from each other, with the second set off-set in a longitudinal direction from the first set. The movable parts are movable relative to the support structure in a radial direction away from the longitudinal axis. A transmission for actuating movement of each movable part of the first and second sets along a predetermined path from the first position to the second position is present. In the expanded state the movable parts are supported against the external load to maintain the respective movable part in its position in the expanded state.

### FIELD OF THE INVENTION

This invention relates to expandable implantable devices for a bone, such as for a vertebra, of a human or non-human mammal. In particular but not exclusively, the invention relates to intra-osseous implants, such as implantable devices suitable for use in percutaneous osteoplasty, such as vertebroplasty and kyphoplasty.

The invention further relates to kits of parts for assembling such an implant, packages with such implants and uses thereof, methods of orthopaedic surgery, surgical tools for expanding implants, surgical tool sets with such tools and kits for orthopaedic surgery comprising such surgical tools or surgical tool sets.

### BACKGROUND OF THE INVENTION

Trauma and other conditions, like osteoporosis or cancer, can lead to parts of bone tissue being weakened, and cause fracture or collapse of the bone. To stabilize the bone and transfer mechanical loads, percutaneous osteoplasty with the injection of bone cement into the bone can be used to stabilize and provide support to the bone. Various types of bones may need such stabilization and support.

For example, spinal fractures such as vertebral body compression fractures frequently result in severe and disabling back pain. Many patients may experience significant morbidity and decreased quality of life secondary to severe pain, prolonged immobilization, kyphosis, pulmonary deterioration, depression, and loss of independence. The most common cause is believed to be osteoporosis. More than 700,000 osteoporosis-related fractures are diagnosed each year in the United States alone. Other causes include primary and metastatic malignancies, trauma, hemangioma and osteonecrosis. In cases where medical therapy, such as exercise, physiotherapy, etc. does not provide sufficient (or any) results in alleviating the symptoms, surgery may be needed.

Vertebroplasty has become a widely used alternative surgical treatment for symptomatic treatment of vertebral compression fractures whose symptoms cannot be treated by medical therapy. Vertebroplasty is a minimally invasive image-guided procedure involving the injection of bone cement into a vertebral body fracture in an effort to reduce pain and improve stability of the fracture. Kyphoplasty is a similar procedure, which utilizes an inflatable balloon in an effort to reduce the fracture and to create a cavity, with the aim of providing a safer injection of cement into the fractured vertebral body.

In these procedures, it is known to use a vertebral body stent which is expanded in the cavity to prevent the vertebral body from collapsing until the bone cement has hardened. However, the commonly known stents are expandable mesh-wire tubular structures (similar to those used in angioplastic stents), and themselves are not capable of withstanding the compressive load acting on the vertebral column. For stabilization and support of the vertebra, the bone cement is thus required.

International patent application publication number WO2010103344 discloses, as an expandable structure alternative to the stent, an expandable implantable device which may be inserted inside a vertebral body, for maintenance and/or restoration of a cavity therein. During surgery, this implant is positioned in the cavity of the vertebra to be restored, and expanded. The device includes a top plate and a bottom plate which can be moved away from each other to expand the stent, and which provide a supporting surface to bear against the bone. A mechanical resistance prevents the expandable implantable device from contracting once it has been expanded. When the device is expanded, the plates thus bear against the bone and support the adjacent bone tissue against the vertical loads acting on the vertebra. Once the device is appropriately positioned, a filler material, such as a bone cement, is injected into the vertebra cavity to fill this void and surrounding bone structures.

A long known, common disadvantage of the existing solutions is that they are only suitable for very specific categories of factures in the vertebra.

For example, Verlaan, J. J., van de Kraats, E. B., Oner, F. C., van Walsum, T., Niessen, W. J., & Dhert, W. J. (2005): “The reduction of endplate fractures during balloon vertebroplasty: a detailed radiological analysis of the treatment of burst fractures using pedicle screws, balloon vertebroplasty, and calcium phosphate cement.” Spine, 30(16), 1840-1845, https://doi.org/10.1097/01.brs.0000173895.19334.e2, discloses that in balloon vertebroplasty a fracture reduction of a burst fracture in an endplate of the human vertebrae is not maintained after deflating the balloon. Verlaan et all. states that this reduction loss might also have been facilitated by the large defects under the endplate that often resulted after maximum balloon inflation and subsequent deflation. Thus, Verlaan evidences that the use of balloon kyphoplasty to treat burst fractures can result in additional defects that weaken the vertebrae, and thus that balloon kyphoplasty is only suitable a specific category of fractures.

Krüger, A., Oberkircher, L., Figiel, J., Floßdorf, F., Bolzinger, F., Noriega, D. C., & Ruchholtz, S. (2015): “Height restoration of osteoporotic vertebral compression fractures using different intravertebral reduction devices: a cadaveric study.” The spine journal: official journal of the North American Spine Society, 15(5), 1092-1098. https://doi.org/10.1016/j.spinee.2013.06.094 discloses an examination of the biomechanical behaviour and height restoration using a device similar to that disclosed in WO2010103344 compared with balloon kyphoplasty in osteoporotic vertebral compression fractures. Krüger et all. discloses that for an anterior vertebral wedge compression with a fractured anterior height of slightly below 70% of the initial height, balloon kyphoplasty only restores the height with about 1%.

Krüger discloses that the balloon expands according to the rules of least resistance and that, in most cases, the balloon touched the lateral wall of the vertebral body before height restoration in the sagittal plane was observed. Thus, use of the balloon is not suitable for fractures in which the part of the vertebra to be restored presents the least resistance and does not have sufficient elastic deformability, such as more complex compression fractures. This is in line with the teaching in Verlaan et al. that the balloon may result in large defects under the endplate. Krüger et. al thus confirms the findings of Verlaan et. all.

For an expandable device similar to that disclosed in WO2010103344, Krüger et all. discloses that the forces of the device work in a craniocaudal direction, that preoperative planning is crucial and that the implant has to be positioned below the upper end plate or above the lower end plate, depending on whether the fracture is a cranial or caudal compression fracture. It thus follows from Krüger that this expandable device is only suitable for fractures where a craniocaudal force can restore one of the vertebral end plates and which allows precise pre-operative planning.

Rotter, R., Schmitt, L., Gierer, P., Schmitz, K. P., Noriega, D., Mittlmeier, T., Meeder, P. J., & Martin, H. (2015): “Minimum cement volume required in vertebral body augmentation—A biomechanical study comparing the permanent SpineJack device and balloon kyphoplasty in traumatic fracture.” Clinical biomechanics (Bristol, Avon), 30(7), 720-725, https://doi.org/10.1016/j.clinbiomech.2015.04.015, equally confirms the findings of Verlaan et all.

In Rotter et al. a wedge compression of the anterior wall of a vertebra was created with a compression of the anterior vertebral edge of more than 40%. Rotter et al. discloses that loss of height is observed in balloon kyphoplasty, which Rotter et al. attribute to a mismatch in the cavity created by the maximally inflated balloon and followed by incomplete cement filling. It thus follows from Rotter et al. discloses that for balloon kyphoplasty does not work well for severe compression fractures.

For the device similar to the one described in WO2010103344, this has only been found to work equivalent to balloon kyphoplasty for patients having one or two painful vertebral compression fracture(s) between T7 and L4, aged <3 months, due to primary or secondary osteoporosis, see Noriega, D., Marcia, S., Theumann, N., Blondel, B., Simon, A., Hassel, F., Maestretti, G., Petit, A., Weidle, P. A., Mandly, A. G., Kaya, J. M., Touta, A., Fuentes, S., & Pflugmacher, R. (2019): “A prospective, international, randomized, noninferiority study comparing an implantable titanium vertebral augmentation device versus balloon kyphoplasty in the reduction of vertebral compression fractures (SAKOS study).” The spine journal: official journal of the North American Spine Society, 19(11), 1782-1795. https://doi. org/10.1016/j.spinee.2019.07.009.

### SUMMARY OF THE INVENTION

The present invention provides implants as described in the accompanying claims. The invention further provides kits of parts for assembling such an implant, packages with implants, uses of implants, methods of orthopaedic surgery, surgical tools for implants, surgical tool sets for orthopaedic surgery and kits for orthopaedic surgery as described in the accompanying claims.

Specific embodiments of the invention are set forth in the dependent claims.

These and other aspects of the invention will be apparent from and elucidated with reference to the embodiments described hereinafter.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 schematically shows cross-sectional views taken along the mid-sagittal plane, of a part of a spinal column of a human in which a first example of an implant is provided, in which:
(A) shows the part of the spinal column with the implant in a non-expanded state, and
(B) shows the part of the spinal column with the implant in a fully expanded state.

FIG. 2 shows various perspective views of a second example of an implant, in which:
(A) shows a perspective view of the implant,
(B) shows an exploded perspective view of the implant,
(C) and (D) show an exploded perspective view of movable parts of the implant, and
(E) shows a perspective view of the internal parts of the implant.

FIG. 3 shows perspective (A), side (B) and cross-sectional (C) views of an example of an implant in a non-expanded state.

FIG. 4 shows perspective (A), side (B) and cross-sectional (C) views of an example of an implant in a first expanded state.

FIG. 5 shows perspective (A), side (B) and cross-sectional (C) views of an example of an implant in a second expanded state.

FIG. 6 shows perspective (A), side (B) and cross-sectional (C) views of an example of an implant in a third expanded state.

FIG. 7 shows various views of a first example of a surgical tool set, in which:
(A) shows a perspective view of the tool set in an assembled state,
(B) shows a cross-sectional view of the tool set,
(C) shows a side view of a surgical driver tool for engaging with a transmission of an implant,
(D) shows a cross-sectional side view, taken along the line D-D in (C) and
(E) shows an enlarged sectional view, corresponding to the part within the circle 7E indicated in (D) and taken along the line E-E in (D), of an engaging end.

FIG. 8 shows a cross-sectional side view of the example of FIG. 2 with the surgical driver tool shown in FIG. 7 engaging thereon.

FIG. 9 shows various views of a second example of a surgical tool set, in which:
(A) shows a perspective view of the tool set in an assembled state,
(B) shows a cross-sectional view of the tool set,
(C) shows a side view of a surgical driver tool for engaging with a transmission of an implant,
(D) shows the engaging end of the driver tool in an enlarged side view, corresponding to the part within the circle indicated in (C), and
(E) shows the engaging end of the driver tool shown in (D), corresponding to the part within the circle 9D indicated in (C) and taken along the line E-E in (C).

FIG. 10 shows a cross-sectional side view of the example of FIG. 2 with the surgical driver tool shown in FIG. 9 engaging thereon.

FIG. 11 shows a flowchart of a method of orthopaedic surgery.

FIG. 12 shows views of a bone in which an example of an implant is anchored, in which:
(A) shows a perspective view of the bone with the implant in the non-expanded state,
(B) shows a top view of the bone of (A),
(C) shows a perspective view of a bone in which an example of an implant is anchored, with the implant in a fully expanded state and
(D) shows a top view of the bone shown in (C).

FIG. 13 shows cross-sectionals view of the bone of FIG. 12 taken along a sagittal plane in respective stages of a method of surgery, in which:
(A) shows the bone prepared for but prior to inserting the implant into the bone,
(B) shows the bone after inserting the implant and anchoring the implant but prior to expanding the implant, and
(C) shows the bone with the implant in an expanded state.

FIG. 14 shows various perspective views of a second example of an implant in which:
(A) shows a perspective view of the implant,
(B) shows an exploded perspective view of the implant, without the support structure which forms an implant body and provides a housing for the internal parts,
(C) shows a perspective view of the internal parts of the implant.

FIG. 15 shows perspective (A), side (B) and cross-sectional (C), taken along the line C-C in
(B), views of the example of FIG. 14 a non-expanded state.

FIG. 16 shows perspective (A), side (B) and cross-sectional (C), taken along the line C-C in
(B), views of the example of FIG. 14 in a first expanded state.

FIG. 17 shows perspective (A) and side (B) views of the example of FIG. 14 in a second expanded state.

FIG. 18 shows a perspective view of the example of FIG. 14 in a third expanded state.

FIG. 19 shows various views of a fourth example of an implant, in which:
(A) shows a perspective view of the implant,
(B) shows an side view of the implant,
(C) shows a cross-sectional view of the implant taken along the line C-C in B.

FIG. 20 shows various views of the example of FIG. 19 in a first expanded state, in which:
(A) shows a perspective view,
(B) shows a top view,
(C) shows a cross-sectional view along the line C-C in (D),
(D) shows a side view, and
(E) shows a cross-sectional view taken along the line E-E in (B).

FIG. 21 shows various views of the example of FIG. 19 in a second expanded state, in which:
(A) shows a perspective view,
(B) shows a top view,
(C) shows a cross-sectional view along the line C-C in (D),
(D) shows a side view, and
(E) shows a cross-sectional view taken along the line E-E in (B).

FIG. 22 shows a perspective view of the example of FIG. 19 in a third expanded state.

### DETAILED DESCRIPTION

Herein below, details will not be elucidated in any greater extent than that considered necessary for the understanding and appreciation of the underlying concepts of the present invention and in order not to obfuscate or distract from the teachings of the present invention.

Referring to FIG. 1, an example of an expandable intra-osseous implant 1 is shown implanted in a bone of a mammal, in this example a vertebra of a human. The implant can for example be a vertebral implant, such as a lumbar, a thoracic or a sacral vertebral implant. The implant 1 may be implanted in-vivo. Throughout this disclosure, the implant 1 may also be referred to as an “implantable device” or simply as a “device”. The device may be used to augment, treat, and/or reinforce a weakened bone to restore the bone tissue and/or to prevent bone collapse, which can occur as a result of osteoporosis or cancer, or heal a bone fracture, such as caused by trauma. In some cases, the bone may have been fractured by an external impact, e.g. by trauma, such as blunt trauma caused by a fall or by a traffic incident or otherwise be weakened and not be able to resist the load.

The shown example is dimensioned to be implanted in an intra-osseous cavity 106 of a bone of a human or non-human mammal, such as in a vertebra. The vertebra may for example be one of the group consisting of: Cervical vertebrae, Thoracic vertebrae, Lumbar vertebrae. The cervical vertebra may for example be one of the group consisting of: C1, C2, C3, C4, C5, C6, C7. The thoracic vertebra may for example be one of the group consisting of: T1, T2, T3, T4, T5, T6, T7, T8, T9, T10, T11, T12. The lumbar vertebra may for example be one of the group consisting of: L1, L2, L3, L4, L5. The cavity 106 may be a void present in the bone prior to surgery. Alternatively, the cavity 106 may be created by a medical practitioner specifically for the implantation of the implant, e.g. by expanding an existing void to be suitable for the implant or creating a completely new cavity. In order to stabilize and support the bone, the implant 1 may be provided in the intra-osseous cavity 106 at an area where the bone is weakened (e.g. fractured). For instance, a patient's bone may have been weakened due to a patient's condition, e.g. osteoporosis or cancer (such as a vertebral tumor or caused by treatment of a tumor outside the spine), and/or has collapsed under the loads that act from the outside on the bone.

The load may be, as in this example, a compressive load. In case of a vertebra this may be the compressive load which the spinal column exerts on the vertebral end plates, as indicated with the vertical arrows in FIG. 1B. The cavity 106 may e.g. be surgically made through a relatively small access passage having a diameter that corresponds to (e.g. is greater than or equal to) the diameter of the device 1 in a non-expanded state. As shown, in the expanded state the diameter of the implant 1 (in this example the dimension in the cranial-caudal direction) is noticeably larger than in the non-expanded state, and accordingly the implant 1 can be brought into the bone with a relatively low invasive surgery, e.g. percutaneously via a passage 104 (not shown in FIG. 1 but e.g. illustrated in FIG. 13) from the exterior of the bone into the cavity. In this example, the passage is a narrow diameter passage which has been created percutaneously. As shown in FIG. 1, that passage can be the same as the passage through which (prior to inserting the implant 1) the intra-osseous cavity 106 has been created but alternatively the implant may be inserted into the intra-osseous cavity 106 via a separate passage. The implant can be implemented to be inserted via a passage prepared before inserting the implant. Alternatively, the implant can be implemented to create the passage upon insertion, e.g. provided with a self-tapping screw body for instance in which the expandable structure 5 is provided.

The implant 1 may e.g. be implemented to be inserted and/or the cavity created via a transpedicular access passage. For example, the transpedicular access passage may extend from an entrance in the lamina or other part of the posterior part of the vertebral arch through the pedicle into the vertebral body. Alternatively, the implant can be implemented to be inserted extra-pedicularly via an extra-pedicular access passage, e.g. such that after implantation a part of the implant abuts, to or extend in a direction parallel to, the side of the pedicle. For example, the extra-pedicular access passage may be located, in circumferential direction of the vertebral body parallel to the vertebral disk, at a side of the pedicle opposite to the side of the pedicle defining the vertebral foramen. The extra-pedicular access passage may for example not pass through any part of the vertebral arch and have the entrance directly into the vertebral body or have an entrance at a side of the pedicle, located in the area between the lamina and the vertebral body and preferably outside the articular facets.

In this example, the implant 1 is a “floating implant” in the sense that it is not anchored to a part of the bone outside the cavity 106 and requires to be fixated in position inside the cavity. The shown example can for instance be placed in the cavity and be fixated in position by expanding the expandable structure 2 until the implant 1 exerts a desired pressure on opposite walls of the cavity sufficient to restrain the freedom of movement of the implant. In this example, the implant exerts the pressure to the upper wall and the bottom wall of the cavity 106. As a result of the expansion, the implant 1 is clamped between the opposite walls of the cavity 106 and thus maintained in position. Alternatively, the implant 1 may e.g. be glued to the walls of the cavity or maintained in position in another manner.

FIGS. 1A and 1B illustrate the implantable device 1 after placement in a vertebra. In FIG. 1A, the implant 1 is shown in a non-expanded state and is positioned close to the upper vertebral endplate 109. In FIG. 1B, the implant 1 is shown after having been transferred from the non-expanded state in an expanded state, in this example a fully expanded state. In this application of the device, by expanding the implant 1, as can be seen in FIG. 1B, the bone may be stabilized against collapse and supported by the implant 1. The vertebra 100 may be lifted to restore its height, at least to a certain extent but preferably completely.

Typical dimensions of the implant 1 (although other sizes are possible as well depending on the cavity in which the device is to be placed, the type of bone and the manner in which the implant 1 is to be implanted in the bone) may be as follows. Device 1 may have a length between 5 mm and 50 mm, such as between 25 mm to 65 mm. For example, the length can be 8 mm or more, such as 15 mm or more. The length can be less than 40 mm, for example less than 30 mm. A suitable range for the length is a length between 8 and 38 mm. A typical maximum non-expanded height, e.g. a diameter, may be less than 8 mm, such as less than 5 mm, such as 4 mm or less. Preferably, although not necessary, the non-expanded height, e.g. diameter, may be 1 mm or more, such as 1.5 mm or more, 2.25 mm or more, such as 3 mm or more. A typical maximum expansion of the device is for example between 1.5 and 4 times the non-expanded height (e.g. diameter). Other maximum expansions are likewise possible, and it is currently preferred that the maximum expansion is less than 5 times the non-expanded diameter which ensures a mechanical stable and reliable expansion.

In the shown example, when the device 1 is positioned in the cavity 106 and when expanded, as described below in more detail with reference to FIG. 2-13, load resisting surfaces 6a-6d may come to contact walls of the cavity 106, and can come to exert a pressure on, e.g. opposite, walls of the cavity 106. The load resisting surfaces 6a-6d are inhibited, in the example completely blocked, by the support structure 2 from moving back to their non-expanded position under the load pressure. Thus, the device 1 provides support to the bone material present between the outside of the bone on which the external load acts and the load bearing surface 6a-6d. Accordingly, collapse of the bone can be prevented. In this example the outside of the bone is the vertebral end plate 109 which interfaces with the inter-vertebral disc 103, and on which the spinal load acts. The device 1 thus supports the vertebra, specifically the vertebral body, to resist the spinal load and to prevent collapse of the vertebra. In some implementations, such as in the examples, the load resisting surfaces 6a-6d can be moved back to their non-expanded position or a less expanded position by operating the device 1 but in an alternative implementation, after expansion the device 1 cannot be collapsed by the operator.

In some implementations, the implant 1 may be expanded until the load resisting surfaces 6a-6d deform and/or displace one or more of the walls of the cavity 106, and e.g. push the bone material forming the wall outwards, in the direction of expansion. Thereby, the outer shape of the bone, in the examples the vertebra 100 can at least partially or completely be restored. In the shown example, the expansion may e.g. partially or completely restore the vertebral height in some or all of the regions of the endplates 109. Alternatively or additionally, for example the implant 1 may be expandable in the direction of the side wall(s) of the vertebral body, such as the anterior part of the side wall, a lateral part of the side walls and/or a posterior part of the side walls of the vertebral body, such as in the examples of FIGS. 14 and 19.

The expandable intra-osseous implant 1 may e.g. be dimensioned for percutaneous placement in a vertebra, such as for placement of the expandable structure in the vertebral body, between the vertebral endplates 108 and 109. For example, the device 1 may have a length in the range of 10 to 70 mm, a width in the range of 3 to 15 mm, a minimum thickness (e.g. when the device 1 is in a non-expanded state) in the range of 3 to 15 mm and a maximum thickness (e.g. when the device 1 is in a maximally expanded state) in the range of 5 to 30 mm.

The intra-osseous cavity 106 can be located close to the surface of the bone on which the external load acts, in this example the vertebral endplate 108,109. For example 5 mm or less, such as 4 mm or less, such as 3 mm or less of bone tissue may be present between a top or bottom load resisting surface 6a,6b; 6c,6d of the implant 1 and the endplate 108,109. This allows an elastic or plastic deformation of this tissue, by a load resisting surface 6a-6d of the implant 1 pushing upon expansion from inside the cavity against the vertebral endplate 108,109. This accordingly allows to reduce the risk of bone fracture or collapse when expanding the implant 1 (e.g. to partially or completely restore the vertebral height). For instance, 1 mm or more, such as 2 mm or more, for example 3 mm of tissue may be present between the (expanded) top side 110 of the implant and the upper (cranial) vertebral endplate 109 and/or between the (expanded) bottom side 111 of the implant and the lower (caudal) vertebral endplate. This reduces the risk that the implant 1 pierces through the tissue and becomes exposed during expansion or post-surgery.

Additionally, as illustrated, the implant 1 may be positioned close to the a side wall, in this example the anterior wall 105 of the vertebra 100. For instance, the implant 1 may be positioned such that there is 1 mm or more, such as 2 mm or more, such as 3 mm or more of space, e.g. with spongy bone material, left between the implant 1 and the wall. Preferably, this space is 10 mm or less, such as 6 mm or less, for example 5 mm or less. This reduces the risk of piercing of the anterior wall 105 by the implant 1. The position of the implant 1 may be determined prior to expansion, for instance, via imaging techniques well known in the art. For example the position may be determined so as to ensure the expandable part of the device is fully inside the vertebral body 102 and e.g. is not in the pedicle, to ensure that an anchoring part is in the pedicle or that the implant is in a desired transpedicular position or an extrapedicular position, for instance. In case the implant 1 is expandable in the direction of the lateral walls of the vertebra to fixate the implant, the distance may be higher to ensure a strong fixation.

The open structure of the implant 1 allows to incorporate the implant 1 in the vertebra 100, i.e. ingrowth of vertebra 100 matter inside the implant 1 can be obtained in addition to bone on-growth on interfaces between the implant 1 and the vertebra 100. More specifically, the solid parts of the structure provide a seed surface for bone material, and, after implantation, form a substrate on which osteoblasts and stem cells can grow. Without wishing to be bound to theory, it is currently believed that the solid parts initially form a seed layer for a cell growth substrate. The cell growth substrate can for example be formed by substances adsorbed to the surface of the solid parts, like proteins, water molecules and/or lipids. Also, the substrate may comprise substances attached to the solid parts of the bulk block, like blood platelets. After formation of the growth substrate, the bone tissue may grow onto the substrate. For example, in case of osseo-integration, osteoblasts or their progenitors, such as osteochondro-progenitor cells or mesenchymal stem cells, will grow thereon and subsequently form the bone matrix in the pores, thus creating an intimate bond between the bone and the implant.

FIG. 2 shows a second example of an expandable intra-osseous implant. Like the first example of FIG. 1, the second example comprises a support structure 2, two or more sets 3,3′ of movable parts 3a, 3b; 3c,3d and a transmission system 7 (not shown in 2A but visible in 2B and 2E). In the following, the implant and its operation are described with reference to the second example, but it will be apparent that this description equally applies to the first example of FIG. 1 and that the expansion of the example of FIG. 1 functions in the same manner. The second example is similar to that of FIG. 1 and differs in that in the example of FIG. 2 instead of being a floating implant the implantable device 1 can be anchored to the bone to fixate the implant in position, as is described below in more detail. More specifically, in addition to an expandable structure 5 to be admitted in the intra-osseous cavity 106, the support structure 2 comprises an anchor body 4, such as a pedicle screw, to anchor the implant 1 in a part of the bone outside the cavity.

In the following, reference is made to the expansion of movable parts 3a-3d. Although strictly speaking a movable part does itself not expand in these examples, a movable part is said to be “expanded” when it is transferred from a first position in the non-expanded state of an implant to a second position in the expanded state of the implant, and when in the second position the movable part projects relative to the shape of the implant in the non-expanded state. A movable part may e.g. be movable along a predetermined path for the movable part, which path extends in a direction of expansion of the implant, from a first location to a second location further away from a centre of the implant. In the examples, the second location is further away from a centre of the implant than the first location and the implant expands by displacement of one or more of the movable parts, e.g. by moving the movable part in a radial direction away from a lateral axis of the support structure. The movable part is therefore in these examples maximally expanded when the second location is the location on the path most remote from the centre.

As explained below in more detail, the implant 1 may be expanded in several ways, which makes the implant suitable for various types of fractures. The implant may for example be used in a patient with for example a spinal condition, such as a thoracolumbar or other spinal fracture. The spinal fracture may e.g. fall one or more categories in the group consisting of: compression injuries, distraction injuries, torsion injuries. More specifically, the patient may have a compression injury which falls one or more categories in the group consisting of: impaction fractures, endplate impaction, wedge impaction, vertebral body collapse, split fractures, frontal split fracture, sagittal split fracture, pincer fracture, burst fractures, incomplete burst fracture, burst split fracture, complete burst fracture, pincer, flexion, axial. Additionally or alternatively, the patient may have a distraction injuries which falls one or more categories in the group consisting of: predominantly transligamentous flexion-distraction injury, predominantly osseous flexion-distraction injury, anterior disruption through the disc. The predominantly transligamentous flexion-distraction injury may for example be with a transverse disc disruption (with or without a flexion subluxation and/or an anterior dislocation and/or with or without fractures of the articular processes, with type A vertebral body fracture. The predominantly osseous flexion-distraction injury may for example be a transverse bi-column fracture, a posterior osseous disruption with transverse disc disruption (e.g. through the pedicles and/or through the interarticular portions (flexion spondylolysis)), with type A vertebral body fracture (e.g. through the pedicles or through the isthmus), anterior disruption through the disc (with or without hyperextension-subluxation, hyperextension-spondylolysis) and/or a posterior dislocation. C: Also, the patient may have a torsion injury which falls one or more categories in the group consisting of rotation-compression injury, rotation-distraction injury, rotational shear injury. A rotation-compression injury may for example be of the impaction type, the split type or the burst type. A rotation-distraction injury may e.g. be with transligamentous flexion-distraction and/or with trans-osseous flexion-distraction and/or with hyperextension-distraction.

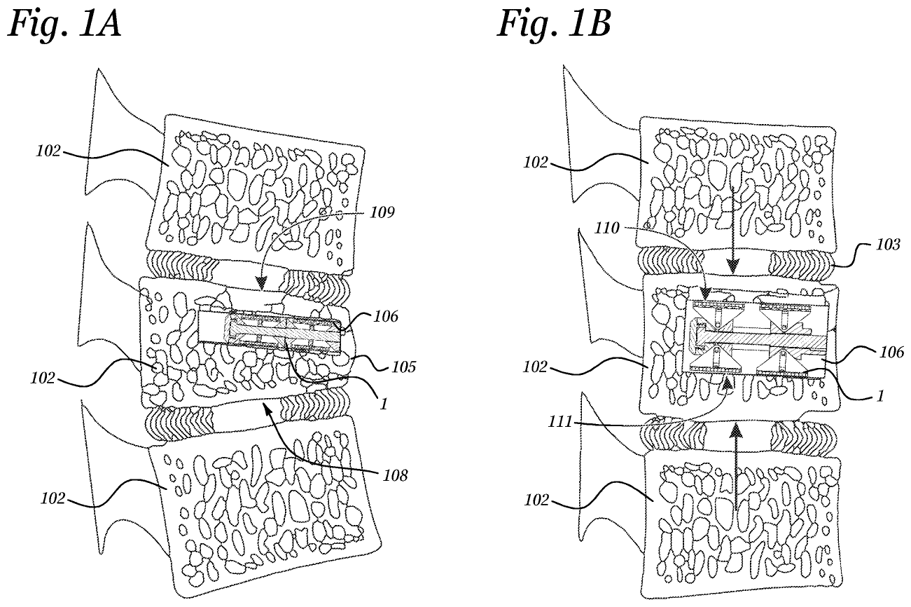

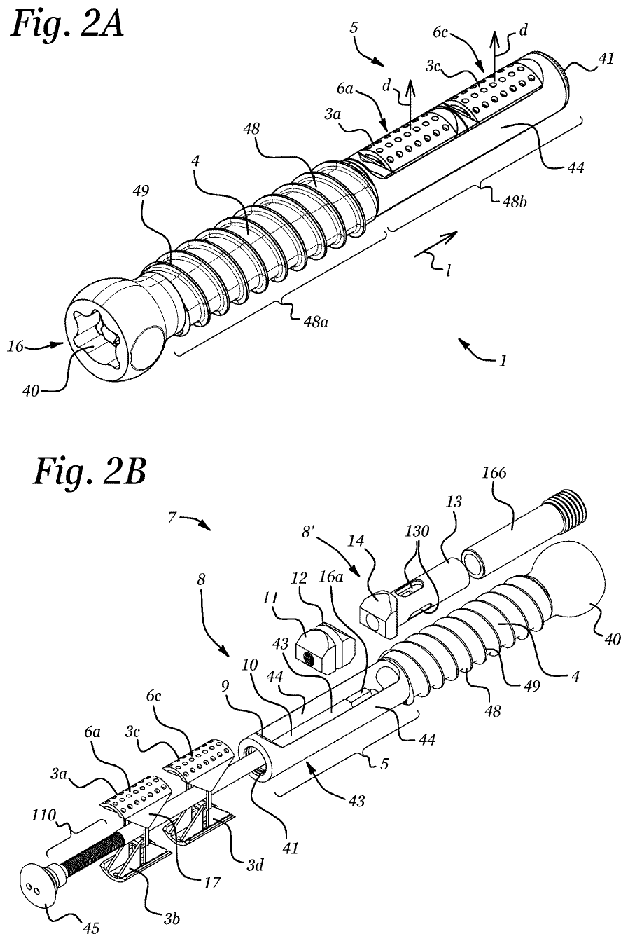

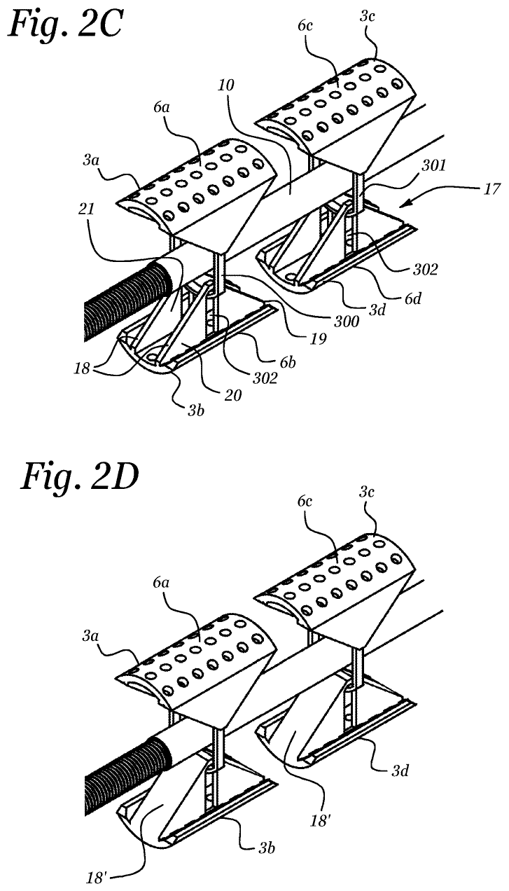

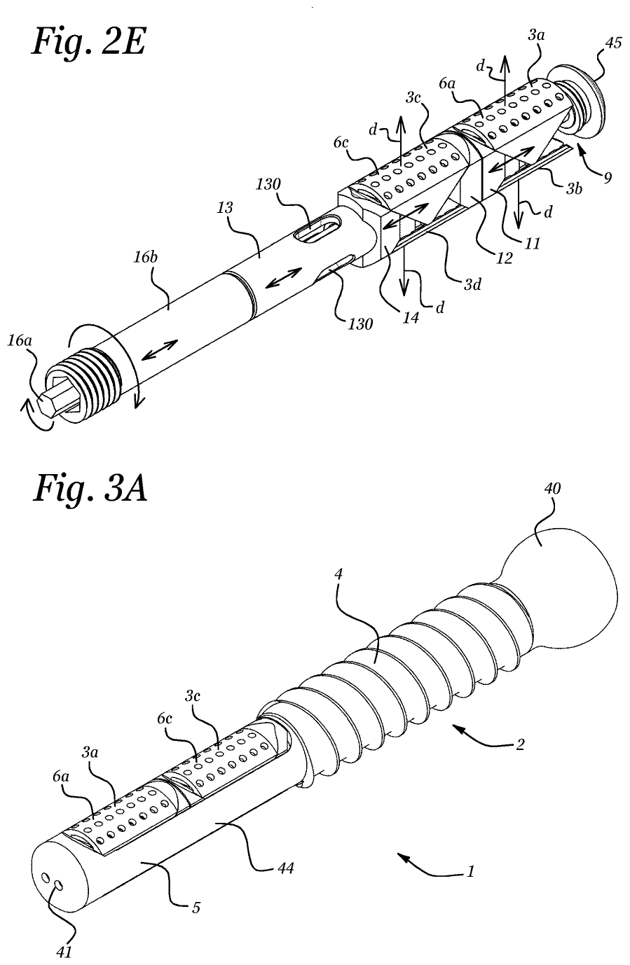

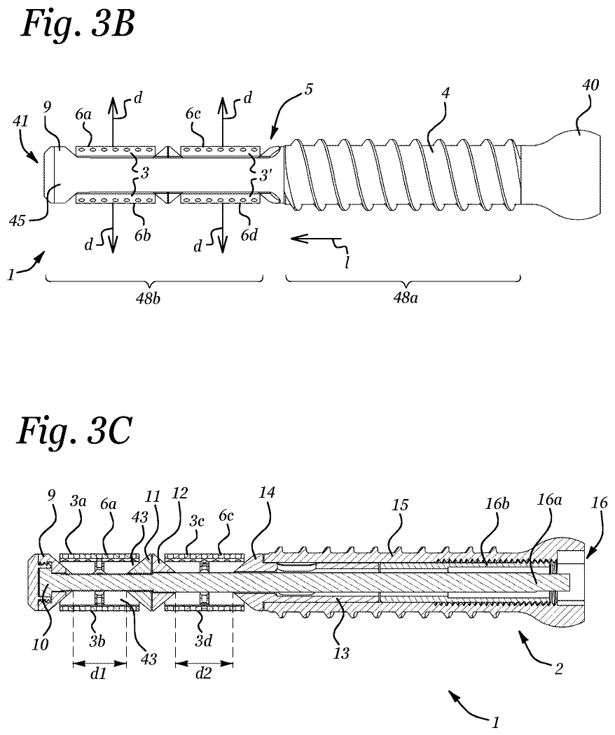

Post-surgery, the implant 1 will normally remain in the expanded state in which the implant has been put by the practitioner, and preferably remains expanded to the extent in which it has been put. That is, post-surgery, the implant 1 remains in that expanded state to stabilize and support the bone, without collapsing under the load. As will be elucidated further with reference to FIGS. 3-6, various expanded states are possible, and the location of movable parts of the device may be adjusted e.g. to have some movable parts at the same location as in the non-expanded state and some of the movable parts moved away relative to the non-expanded state. For example, instead of all, only some of the movable parts may be expanded.

The implant 1 comprises two or more sets 3,3′ of movable parts 3a,3b; 3c,3d. The sets 3,3′ of movable parts may be expanded to different extents. The amount of expansion, as well as which movable parts are moved, may be determined by the healthcare practitioner (e.g. a surgeon performing the surgery), such as based on the patient anatomy and/or the nature of the cause, size and/or location of the weakening in the bone being treated. In this example, the expandable structure 5 has a non-expanded state and one or more expanded states.

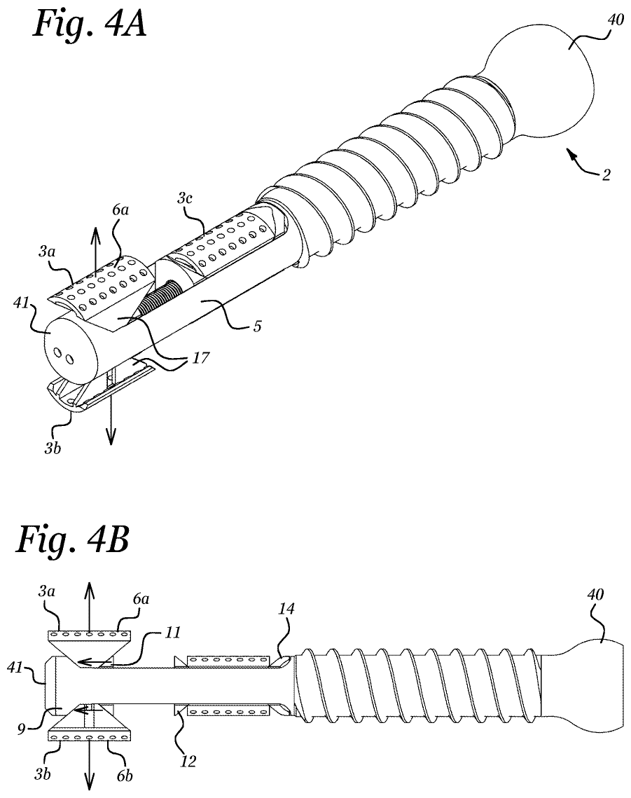

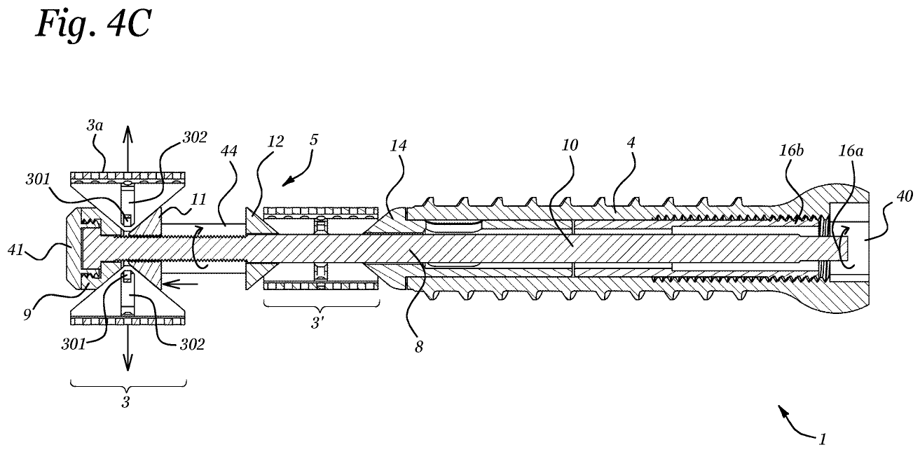

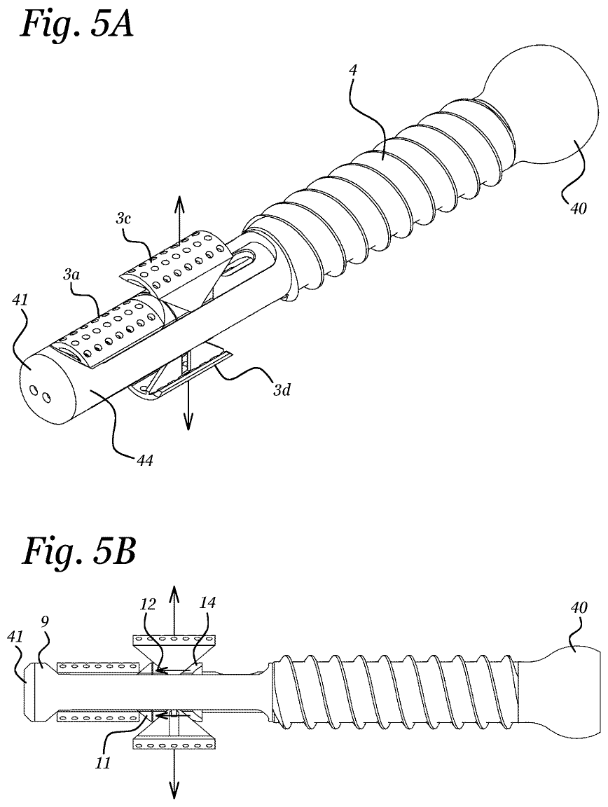

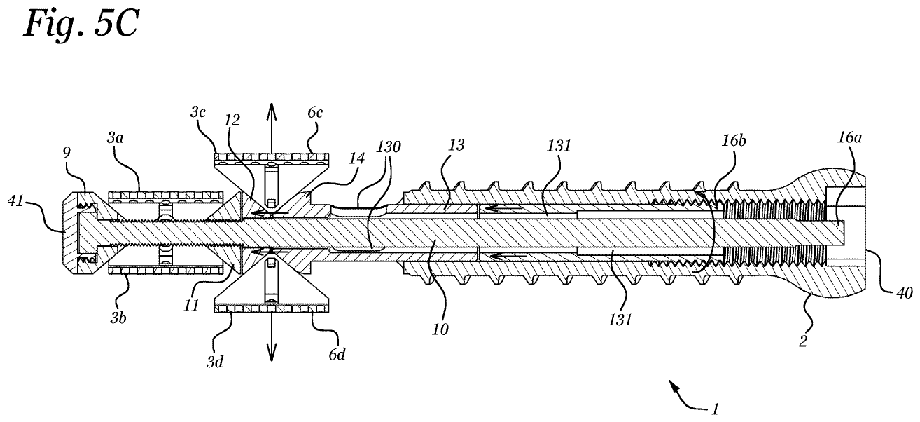

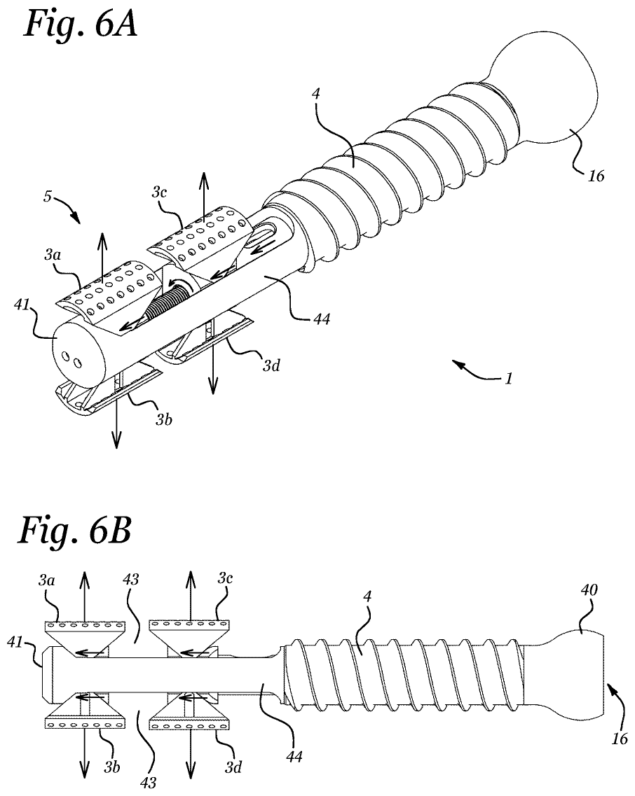

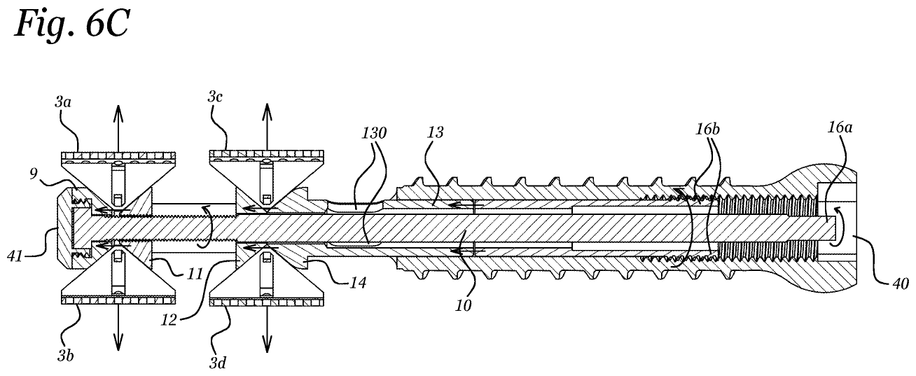

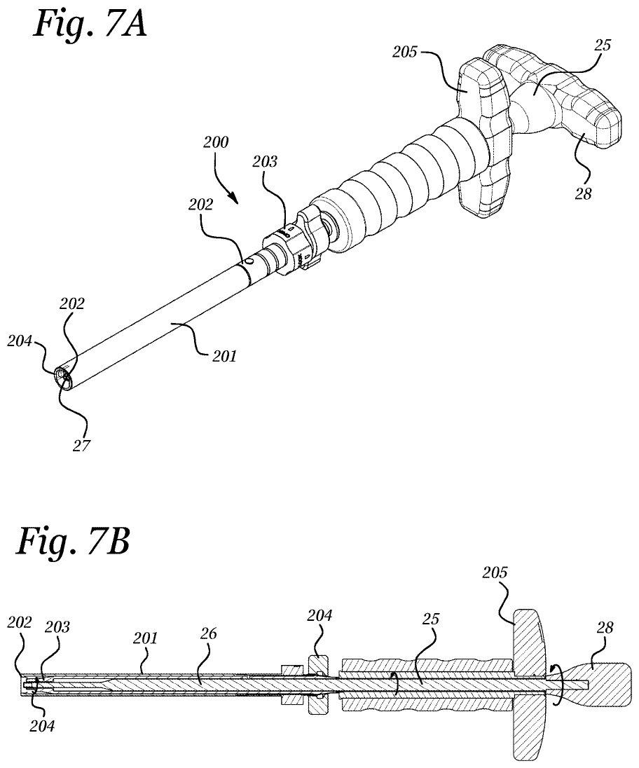

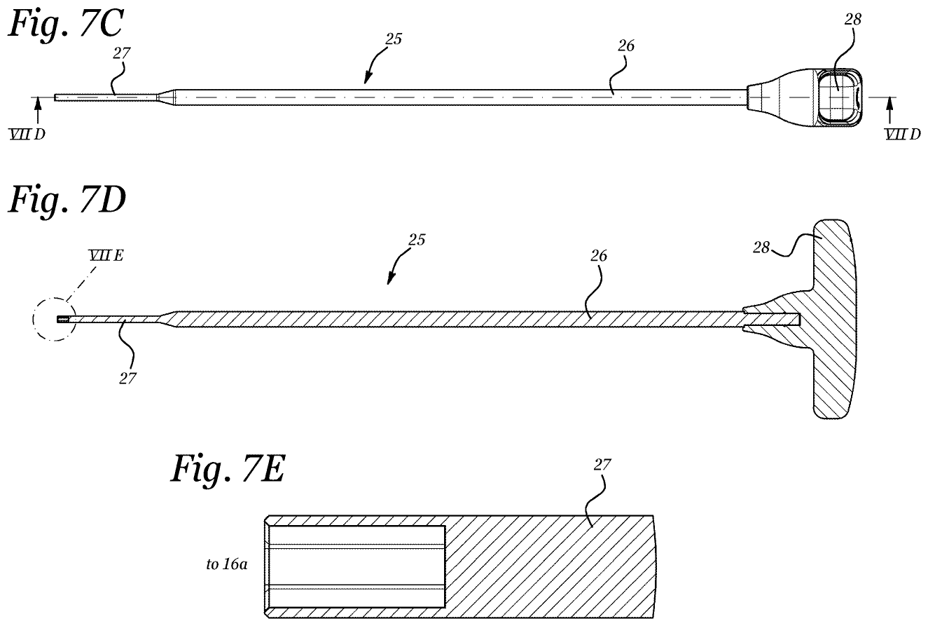

The sets 3,3′ are in this example selectively expandable. The implant 1 may have a fully expanded state, in which all movable parts 3a-3d of all sets 3,3′ are moved to a position in which the movable part maximally projects relative to the shape of the implant in its non-expanded state. The implant 1 may have one or more partially expanded states in which only a subset of the sets 3,3′ is expanded. For example, in some implementations, one set 3 of movable parts may be expanded (e.g. as shown in FIG. 4) and a second set 3′ of movable parts may remain in a non-expanded state or visa-versa (e.g. as shown in FIG. 5) or two or more, such as all, sets may be expanded (as shown in FIG. 6). In an expanded set, for instance, all movable parts of that set may be expanded.

Although the first, second and third expanded state illustrated in FIG. 4-6 are states in which one or more of the sets 3,3′ of the implant 1 are maximally expanded, the movable parts 6a-6d of a set 3,3′ of the implant 1 do not need to be expanded to their maximum expansion in an expanded state. As explained below, each set 3,3′ of the implant may have other expanded states between the non-expanded state and the state with maximal expansion of that set. For instance in the examples in the illustrated expanded states the movable parts of the expanded set or sets 3,3′ are shown at their maximum distance, away from a centre of the implant (formed in this example by the longitudinal axis of the elongate support structure 2). The medical practitioner may deemed a lesser degree of expansion for the chosen expanded state suitable to provide the stabilization and support considered necessary and set the implant accordingly. Depending on for instance the characteristics of the bone, the support required and the load conditions, a healthcare practitioner may elect to move one or more of the movable parts of the implant 1 to its maximally expanded position or to a lesser expanded position. In the examples, the movable parts 3a-3d are only movable in one direction, here radially, away from a longitudinal axis of the implant and maximally expanded when they are in at the location radially most remote from the longitudinal axis. In this example for instance, one or more of the movable parts 3a-3d may be placed at a location closer to the longitudinal axis than the radially most remote location and the set 3,3′, and thus be expanded to a degree larger than no expansion and less than maximal expansion.

One or more, e.g. each, set 3,3′ may be infinitely adjustable, that is have a continuous range of expanded states between the non-expanded state and its maximum expansion in which one or more of the movable parts of the implant 1 are partially expanded, but not to the extent required to provide the stabilization and support considered necessary by the healthcare practitioner. In such a case, the device will pass through intermediate states in the transition from the non-expanded state and the expanded state with the degree of expansion of chosen by the practitioner. Alternatively, set 3,3′ of the implant 1 may have a discrete number of expanded states and e.g. be configured to maintain a movable part 3a-3d against the load in position only at spaced apart locations on the predetermined path.

The expandable intra-osseous implant 1 has a non-expanded shape in the non-expanded state and an expanded shape in each of the expanded states of the implant. The expanded shape projects compared to the non-expanded shape of the implant in one or more directions of expansion. A side of the implant 1 at which the implant 1 is expandable and at which in the expanded state projects relative to the non-expanded shape is hereinafter referred to as an “expansion side”. In this example for instance, the support structure 2 defines a longitudinal axis and the implant 1 is provided with movable blocks in some regions of the lateral sides. The movable blocks are movable in a direction extending away from the lateral sides of the implant body formed by the support structure 2. The implant 1 has a proximal end and a distal end, which respectively coincide with the proximal end 40 and distal end 31 of the anchor body 4 in this example. The expansion side lies between the proximal end 40 and the distal end 41, and the direction of expansion extends perpendicular to a proximal-distal direction from the proximal end 40 to the distal end 41, in the radial direction. The implant 1 may have one, or more than one, such as two or four expansion sides, at which the implant 1 can have an expanded shape which projects compared to the non-expanded shape of the implant in a direction of expansion extending away from the expansion side. In some implementations, the implant 1 may for example have at least two expansion sides facing away from each other, e.g. such that the direction of expansion of the first expansion side is opposite, or substantially opposite, to the direction of expansion of the second expansion side. As can be seen in FIGS. 3C, 4C and 5C for instance, in the second example the implant has two expansion sides. The second example is expandable in two first expansion directions extending in opposite, but in this example parallel, directions. In addition or alternatively, in some implementations, the support structure 2 may comprise at least two expansion sides which are perpendicular, or substantially perpendicular to each other, e.g. such that a first direction of expansion of the first expansion side is orthogonal, or substantially orthogonal, to a second direction of expansion of the second expansion side. The implant can e.g. be expandable in two second expansions direction extending away from the first expansion direction. In the third and fourth example for instance the implant is in addition to the first expansion direction, expandable in two second expansion directions extending in opposite, but in this example parallel, directions which extend away from, e.g. are perpendicular to, the first expansion directions.

In some implementations, the direction of expansion may be perpendicular to a longitudinal axis of the device 1. In the shown example, the device 1 is for instance cylindrical or tubular (or substantially cylindrical or tubular) in shape and the directions of expansion extend in radial direction away from the longitudinal axis of the cylindrical or tubular implant body. In some implementations, the direction of expansion may be perpendicular, or substantially perpendicular, to the outer surface of the implant body in the expansion area. In the shown implementations, for instance, the longitudinal axis of the implant extends when properly positioned more of less parallel to the vertebral endplates. The implant is expandable in a vertical direction, towards the vertebral endplates to push the bone material outwards and support the vertebral endplates against the load acting on the spine of the mammal, (e.g. to partially or completely restore the vertebral height) as is elucidated below in more detail with reference to FIGS. 12-13. Said differently, in such implementations, the implant 1 can expand when correctly positioned in a human vertebra in the cranial-caudal direction (at the side shown in the FIGs. as the bottom side 111 of the implant 1) and in the caudal-cranial direction (at the side shown in the FIGs. as the upper side 110 of the implant).

The support structure 2 may be implemented in any manner suitable for the specific implementation. When the implant is in the expanded state, this support structure 2 supports each of the movable parts 3a-3d against the load to maintain the respective movable part 3a-3d at its second location. Said differently, the support structure 2 maintains the implant 1 in expansion against an external load.

Still referring to FIG. 2, in the second example, the support structure 2 forms the implant body. Although other shapes may be used, in the present example, the body of the device 1 has a generally circle cylindrical shape, but alternatively this may be e.g. a non-circular elliptical cylindrical or other suitable cylinder, such as a hexagonal or other polygonal cylinder, and generally another elongated shape, such as a fusiform, cigar-shape or lemon-shape or a rectangular cuboid. Preferably the shape has chamfered or fillet edges. A first length of the body, from the proximal end 40 to a certain point at a distance from the proximal end 40 and the distal end 41, is provided with the bone anchor 4. In this example, the first length extends from the proximal end 40 to the distal side end of a profiled area 48 provided on the outside of the body 4. A second length of the body from the distal end 41 to a certain point, in the direction towards the proximal end 40 at distance therefrom, is provided with the expandable structure 5. In an alternative implementation, e.g. as illustrated in FIG. 1, the first length may be absent and the implant 1 without bone anchor for instance. For example, the profiled area may be omitted.

The support structure 2 is provided with an expandable structure 5 to be admitted in the intra-osseous cavity. In this example, the support structure 2 is shaped as a housing with a hollow, substantially tubular inside. The tubular inside may also be referred to as a bore. In this example the tubular inside has a round shape but in other implementations e.g. square, rectangular or other hollow passages may be used. The outside has a substantially similar cross-sectional shape in this example, and the support structure 2 may be referred to as a pipe or tube. The expandable structure 5 is provided in the housing and expandable in the direction of expansion through slots in the housing. In this example, each slot forms an opening which connects the hollow inside with the outside of the housing, as can be seen in FIGS. 3C,4C,5C for example. In this example, the slots are radial passages in the longitudinal wall of the tube-shaped housing. Although other implementations are possible, in this example in the hollow inside of the housing first and second transmissions 8,8′ of a transmission system 7 are admitted. In this example the transmission system 7 extends through the tubular body, more specifically in the longitudinal direction from an opening at the proximal end 40 towards the distal end 41. The support structure 2 holds the transmission system 7 in position. In this example, the transmission system 7 in turns holds the movable parts 3a-3d in position.

The expandable structure 5 may comprise at least two sets 3,3′ each comprising one, two or more movable parts 3a-3d, such as a plate or a bulk block, each with a load resisting surface 6a,6b; 6c,6d for supporting a wall of the intra-osseous cavity against a load acting on the bone. The movable part 3a-3d is movable away from the support structure, in this example from the housing, in a direction of expansion d. This direction of expansion d is in this example perpendicular to the longitudinal direction I, from the distal end 41 towards the proximal end 40. The displacement of thae movable part 3a-3d brings its load resisting surface 6a,6b; 6c,6d from an initial position in the non-expanded state, shown in FIGS. 2 and 3 to an expanded position in an expanded state, such as shown in FIGS. 4-6. When correctly positioned in the cavity, the load resisting surface 6a,6b; 6c,6d then abuts to the wall of the intra-osseous cavity and supports the bone matter from which the wall is made, e.g. against a load acting thereon from outside the bone, such as a compressive load acting on an outside surface of the bone in a direction opposite to the direction of expansion. The implant has a maximal expansion but does not need to be expanded to the fullest extent. In the second example, the displacement of the load resisting surface 6a,6b; 6c,6d is constrained to a limited range, the limited range being between the initial position in the non-expanded state and a maximally expanded position, and the position is infinitely adjustable between the initial position and the maximally expanded position by a suitable actuation of the movement, as will be apparent from the below.

At the expansion side, the support structure 2 may have an expansion area, the expansion area being a portion or region of the support structure 2. That is, at the at least one expansion side the expandable structure 5 may be provided with one or more movable parts 3a, 3c. In some implementations, the housing may at the expansion side have a curved or flat surface with one or more openings or recesses in which a plurality of movable parts 3a, 3c is received, e.g. partially or completely recessed in the structure 2 in the non-expanded state, as shown in FIG. 2A for instance. For example, in implementations in which the expandable structure 5 is in the non-expanded state cylindrical, tubular, substantially cylindrical or substantially tubular, at expansion side a recess, a cut-out or groove may be provided within an outer wall of the housing formed by the support structure 2. In FIG. 2A, for instance, openings 43 extend longitudinally along support structure 2 and span a fraction of the circumference of the device 1, e.g. extend in the circumferential direction around the longitudinal axis over less than 90 degree, e.g. less than 60 degrees, e.g. less than 30 degrees.

Still referring to FIG. 2, the device 1 comprises at least two sets 3,3′ of movable parts. Each set 3,3′ comprises at least one movable part 3a,3b; 3c,3d. As elucidated below in more detail, each movable part 3a,3b; 3c,3d of a set 3,3′ can be actuated by the transmission system 7 to move relative to the support structure 2 and to relative the movable parts of the other set(s). Thereby, the expansion of the implant can be adapted to different types of fractures by moving selected movable parts, in an order and to an extent deemed suitable by the medical practitioner.

The sets may be implemented in any manner suitable for the specific implementation. The implant may comprise two or more sets 3,3′. Each set 3,3′ may comprise one, or more than one movable parts. For example, each set may have two, three or more movable parts. Each set may have a movable part at an expansion side which is also an expansion side of another movable part of the other set and/or a movable part at an expansion side which is not an expansion side of another movable part of the other set. For example, the movable parts may in the circumferential direction overlap, and a movable part may have a direction of expansion which extends in a direction parallel to that of that other movable part.

In the simplest implementation, each set 3,3′ comprises a single movable part and both sets are located at the same side of the support structure. In that case the implant 1 has two blocks at the same side of expansion which are movable relative to each other. For example, the implant may have a single expansion side provided with the movable parts of the sets 3,3. In the second example though, the implant 1 comprises two or more sets 3,3′ of movable parts of which each set 3, 3′ comprises two, or more than two, movable parts 3a,3b; 3c,3d. Here, seen in the circumferential direction around the longitudinal axis, the movable parts of a set are located at a different position. That is, in each set 3,3′ the movable parts 3a,3b; 3c,3d are circumferentially spaced around the longitudinal axis of the support structure 2. There may be an overlap in the circumferential direction around the longitudinal axis between successive movable parts, e.g. in case the movable parts of a set are distributed in the longitudinal direction. However, in the examples there is in the circumferential direction a spacing between the movable parts of each set and the movable parts of the set are spaced apart in that direction. The movable parts of a set overlap in this the longitudinal direction and more specifically are located at the same position from the distal end 40. In the shown examples, seen in the proximal-distal direction, also referred to as the longitudinal direction I, each set 3,3′ of movable parts is located at a different distance from the proximal end 41. That is to say, each set 3,3′ of movable parts is longitudinally offset relative to the other set.

In some implementations, there is at least one movable part from each of the sets 3,3′ located at each of two opposite expansion sides. In the first and second example, two sets 3,3′ are present and each set comprises a top-side movable part 3a,3c and a bottom side movable part 3b,3d. If the implant is correctly positioned, the top-side movable part 3a,3c faces the upper vertebral endplate 109, and is expandable towards the upper endplate 109, while the bottom side movable part 3b,3d faces the lower vertebral endplate 108 and is expandable towards the lower vertebral endplate 108, in a direction opposite to the direction of expansion of the top-side movable parts 3a,3c.

In some implementations, the direction of expansion of the movable part in one set extends in the same direction, e.g. is parallel to, as the direction of expansion of the movable parts of the other set(s). In the second example, two sets 3,3′ are present, with a first set 3 having directions of expansion parallel to the directions of expansion of the other set 3′ but in alternative implementations the angle may be more than 0 degrees, and preferably less than 45 degrees. In some implementations, the direction of expansion of the movable part in one set 3 extends away from, e.g. is perpendicular to, the direction of expansion of the movable parts of the other set(s) 3′. In the third and fourth example, two sets 3,3′ are present, with a first set 3 having directions of expansion perpendicular to the directions of expansion of the other set 3′ but in alternative implementations the angle may be less than 90 degrees, and preferably more than 45 degrees. In a currently preferred implementation, the implant 1 has expansion sides which comprise or consist of at least two opposite first sides of expansion, and optionally at least two opposite additional sides of expansion perpendicular to the first sides.

The sets 3,3′ may comprise a first set 3 of one, or more than one, load bearing movable part 3a and one, or more than one, shielded movable part 3b. In the second example, the sets comprise a second set 3′ of one, or more than one, load bearing movable part 3c and one, or more than one, shielded movable part 3d. When implanted, the load resisting surfaces 6a,6c of the load bearing movable parts 3a,3c face towards the load, which in this example acts in the cranial-caudal direction due to gravity. Their load resisting surfaces resist the load, and therefore shield the bone and the movable parts which in the direction of the load are further way from the outside of the bone on which the load acts, in this example “further way” is below the load bearing movable parts. The shielded movable parts in turn have the load resisting surfaces oriented facing away from the load and resist the counter force induced by the load and thereby support the implant 1. In addition to supporting the bone, the movable parts 3a-3d resist a load and thus allow to reduce the load pressure on the material in the cavity 106. This can shield the cavity 106 and help healing of the bone because by this shielding bone regrowth in the cavity can be improved. Thus, the load resisting surfaces support the bone matter against the load, and shield the volume of the cavity separated from the bone matter by the load resisting surfaces, from the load. Alternatively or additionally, such as in the third and fourth example, the sets 3,3′ may comprise a second set 3′ of one, or more than one, side-wards movable part 3c, 3d which is movable in a direction perpendicular to the load bearing and shielded movable parts of the first set 3. The side-wards movable parts can e.g. resist a load in the side-wards direction or be used to clamp the implant 1 in the cavity, either by themselves or in cooperation with the bone anchor 4.

As indicated with the arrows in the FIGs, the movement of the movable parts 3a-3d can be a translational movement, and more specific a rectilinear movement,. In alternative implementations the movement can be a translational-rotational movement or a curvilinear movement. As illustrated with the arrows, the paths along which the movable parts move are parallel in the shown examples. However, in an alternative implementation, for instance, a first part may in addition to moving in the direction of expansion move towards the distal end 41 and/or another part may in addition to moving in the direction of expansion move away from the distal end 41, and the paths e.g. diverge in the direction of expansion. For example in the third example, as can be seen in FIG. 21C, the movable parts of each set more both in the radial direction and the longitudinal direction of the implant when expanded. However, other non-parallel paths are also possible, such as curvilinear paths which respectively curve towards and away from the distal end.

A movable part may be implemented in any manner suitable for the specific implementation. Some or all of the movable elements 3a-3d may be made of a deformation resistant material. That is, the movable element 3a-3d may be rigid (e.g. sufficiently rigid to resist deformation under the load), e.g. shape-retaining under the load. Alternatively or additionally, some or all movable elements 3a-3d may bend, such as elastically deform (that is flex) under the load. For example, load resisting surface 6a-6d of that movable element 3a-3d may bend, e.g. flex under the load. For instance, the load resisting surface 6a-6d may bend, e.g. flex, under the load around an axis parallel to the load resisting surface 6a-6d and non-parallel to the longitudinal direction of the implant 1. Instead of flexing, bending with a plastic deformation is likewise possible.

In the examples, each movable part 3a-3d comprises a load resisting surface 6a-6d for resisting a load extraneous to the device 1. Each movable part 3a, 3b is movable to position its load resisting surface 6a, 6b, relative to the support structure 2 and the other movable parts of the set 3 of movable parts. That is, each movable part may be movable along a predetermined path to bring its load resisting surface to a desired position (e.g. a position as determined by a healthcare practitioner operating the device 1, e.g. surgeon). Each movable part 3a, 3b may be maintained in a particular position and/or location, for example by a transmission (described below) and the support structure. Each movable part 3a, 3b may be maintained in a position against the load.

The load may be a load acting in a direction opposite to the direction of expansion. The direction of expansion of a movable part 3a-3d may be perpendicular or substantially perpendicular to the plane of the respective load resisting surface 6a,-6d of the movable parts 3a-3d. The load resisting surface 6a-6d may be a surface of a movable part 3a-3d facing the direction of expansion of the respective part, in the examples facing radially outward from the support structure 2.

As is best seen in FIG. 2C and D, each of the load resisting surfaces 6a-6d may have an elongated shape which extends in its longitudinal direction from a first transversal end to a second, opposite transversal end, with lateral edges between the transversal ends defining the lateral sides of the load resisting surface 6a-6d. However, other shapes (e.g. elliptical or other) are also possible. The load resisting surface 6a-6d may be coplanar with the surface of the support structure 2 in the non-expanded state of the implant 1.

In some implementations, the load resisting surface 6a-6d may be an outer surface of the movable part 3a-3d. As can be seen in FIGS. 2A,14 and 19A for instance, the movable parts 3a,3c, may be placed with their load resisting surfaces 6a,6c forming in the non-expanded state a continuation of the outside of the body of the device 1, in circumferential direction around a longitudinal axis of the body of the device 1. In the shown example, in the non-expanded state the load resisting surfaces 6a,6b; 6c,6d each form an exposed region at the outer surface of the body of the device 1. In FIG. 2 the body is formed by the support structure 2 and the exposed regions are located outside the anchor body 4, in the region between the anchor body 4 and the distal end 41. The load resisting surfaces are in the non-expanded state part of the expandable structure 5 comprised in the support structure 2. When the respective movable part 3a-3d is expanded the load resisting surface 6a-6d is located in the, in this example radial, direction of expansion at a distance from the expandable structure 5.

In the shown implementations, the load resisting surface 6a,6b; 6c,6d is a non-planar surface, and more specifically is a bend surface. Each surface can e.g. be convex and be curved to define a concave space with the open side facing away from the direction of expansion, in the example the concave space faces towards the longitudinal axis. The load resisting surface may e.g. be curved in a circumferential direction around the longitudinal axis of the support structure 2, which corresponds to the transversal direction of the load resisting surface 6a,6b; 6c,6d in this example. In the example, the surfaces are flat in the direction of the longitudinal axis. Alternatively, the load resisting surface 6a,6b; 6c,6d may have another dome-shaped curvature, like a cap (such as an ellipsoid cap) and for example be curved in two directions, e.g. in the longitudinal direction as well as the circumferential, or be partially flat, for example have chamfered edges or be provided with a flange, or be completely flat, just to name a couple of examples.

The load resisting surfaces 6a-6d of two or more movable parts 3a-3d may be parallel. That is, the load resisting surfaces of two, or all, movable parts in a set may be parallel and/or load resisting surfaces of two, or all, movable parts in a set may be parallel to load resisting surfaces of two, or all, movable parts in another set. In this respect, in case of curved surfaces, the term “parallel” is understood to mean that the axes around which they are curved are parallel and a tangential line exists which for all surfaces is perpendicular to the axes and to the directions of expansion. In some implementations, load resisting surfaces 6a, 6b of movable parts 3a, 3b within a set 3 of movable parts may be non-parallel. That is, the load resisting surfaces of two, or all, movable parts in a set may be non-parallel. In some implementations, the angle of a load resisting surface 6a-6d relative to the direction of expansion may be adjusted, e.g. prior to insertion into a bone, or after insertion into a bone. For instance, the movement of the movable parts may be a translational-rotational movement or a curvilinear movement.

The load resisting surface 6a-6d of a movable part 3a-3d may be porous. That is, the load resisting surface may be provided with one or more holes, or pores, as can e.g. be seen in FIGS. 2C and 2D. Although the surface may in addition be provided with blind pores, in the implementation the load resisting surface 6a-6d is only provided with through-pores, which are in fluid communication with a space shielded from the load by the load resisting surface in the expanded state of the implant, in the examples between the surface 6a-6d and the axis of the support structure. Such an open structure facilitates incorporation of the implant 1 in the bone, e.g. vertebra 100. That is, ingrowth of vertebra 100 matter inside the implant 1 can be obtained in addition to bone on-growth on the load resisting surface 6. In this respect, although the Figs. show an enlarged view, it in reality the device 1 will be dimensioned to fit into a bone of a mammal and may be sized to be provided through a minimal invasive procedure. Accordingly the openings in the load resisting surface will have such a small pitch that they act as pores. Thereby the device 1 can operate as a scaffold for the bone tissue. In this respect, the device 1 can be osteoconductive and/or osteoinductive. The load resisting surface may have an openness which is more than 0%, such as at least 10%, such as at least 20%, such as at least 30%, such as at least 40%, for example at least 50%, such as at least 55%. Currently most preferred is an openness in the range of 50% to 80%. The average distance between pores can e.g. be between 50% and 200% of the average pore diameter. The average distance can e.g. be isotropic or anisotropic.

Specifically referring to FIG. 2C and D, as shown, one or more, e.g. each, movable part 3a-3d may comprise a base 17. In the example the base 17 extends from the load resisting surface 6a-6d in a direction opposite to the direction of expansion, here towards the longitudinal axis of the support structure 2. The movable part 3a-3d may be arranged within the implant body, in this example the support structure 2, with the base 17 in the non-expanded state being partially or completely recessed in the body and extending from the load resisting surface towards the inside of the body. In the example, the load resisting surface 6a-6bd is in the non-expanded state flush with the cylindrical body and the base completely recessed. The base 17 may have lateral walls 20,21, which for instance may be triangular or substantially triangular (e.g. truncated or having curved sides) in shape with base proximal to the load bearing surface and the apex distal from the load bearing surface 6a-6d. The base 17 may comprise sides extending between the walls 20,21, from the top towards the bottom, with a second side 19 being an opposite side to a first side 18. In the examples the walls 20,21 extend in the longitudinal (axial) direction and the sides 18,19 extend in the radial direction perpendicular to the longitudinal direction. The first side 18 and/or the second side 19 may be inclined and e.g. be flat or be curved and have a convex or concave shape.

The base 17 may be hollow or substantially hollow, e.g. a frame. The base 17 may have an openness, i.e. the aggregate volume of the base occupied by the structure of the base relative to the total volume of the base of at least, or equal to one of the group consisting of: 70%, 80%, 90%, such as less than one of the group consisting of: 95%, 85%, 75%. In some implementations, the base 17 may be solid, e.g. not hollow, e.g. a block. The base 17 may for example comprise an open, inner cavity. For instance, base 17 may taking the load resisting surface 6a-6d as its, closed, top, be open at the bottom. In FIG. 2C for instance, spaced apart walls 20,21 extend from the top and define an open, inner cavity which is open at the bottom, and which in the implementation of FIG. 2C is open at the sides 18,19 as well. In an implementation, the distance between the walls 20,21 differs between movable parts, such that first wall 20 and second wall 21 of a first movable part fit in the open, inner cavity of a second movable part of the same set or of another set. In FIG. 2C for instance, the width of the load bearing movable part in one of the sets is equal to, or less than the width of the shielded movable parts in the set. Said differently, the load bearing movable parts 3a,3c and the shielded movable part 3b,3d slide into each other, such that their walls overlap in the radial direction in the non-expanded state. This allows to obtain a reduction in diameter of the implant in the non-expanded state and therefore to reduce the invasiveness of the procedure required to place the implant into the mammalian body in-vivo.

A second implementation of the movable parts is illustrated in FIG. 2D. The implementation shown in FIG. 2D differs from that shown in FIG. 2C in that the first side 18 and/or the second side 19 of the shielded movable parts 3b,3d has a closed surface 18b′18d′, e.g. the space between the walls 20,21 may be filled and the base 17 be solid. The distance between the walls 20,21 of the load bearing blocks is such that they can slide over the base 17 of the shielded movable parts 3b,3d.

As can be seen more clearly in FIGS. 3C,4C and 5C, the implant 1 comprises a transmission system 7 for actuating movement of each movable part 3a,3b; 3c,3d of the sets 3,3′ from the first position to the second position, with the movable parts supported in the expanded state against the external load to maintain the respective movable part in its second position. As will be apparent from the below, the implant 1 has a relative low risk of failure because the mechanical construction is relatively simple. The associated risk of movable parts being jammed during insertion or expansion is therefore reduced as well.

The movement of a movable part may be relative to the support structure 2 and/or relative the movable parts of the other sets 3,3′ and/or other movable parts of the same set. In the shown examples, the movements of the movable parts in the same set 3,3′ are coupled to each other and all movable parts in that set will expand to the same extend. The movement may be coupled such that the movable parts of a set expand either in the same or parallel but opposite directions. Alternatively, the transmission system 7 may be implemented such that within a set of movable parts, each movable part may be expanded to a different extent, e.g. the movable parts in a set of may be expanded independent from each other or the transmission system 7 may drive different movable parts in the same set with a different transmission ratio but still dependent on each other. Once expanded, the state of the implant 1 may be finalized, e.g. by securing the parts of the implant 1 moved to expand the implant 1, e.g. by gluing them to the walls of the cavity.

The transmission system 7 may be configured such that the sets 3,3′ can be selectively moved to control the shape into which the implant 1 expands. The shape of the implant can thus be adapted to various types of fractures, as deemed suitable by a healthcare practitioner for the type of bone, condition of the bone and/or type of fracture for instance. For example, in a first expanded state the first set 3 is expanded and the other sets remain unexpanded. In a second expanded state, a second set 3′ is expanded and the other sets remain unexpanded. It will be apparent that this concept may be extended to e.g. three or more sets. In a third expanded state multiple, e.g. all, sets are expanded and the implant is then in a mixed expanded state which e.g. comprises both the first expanded state and the second expanded state. In case three or more sets are present, the implant may have multiple mixed expanded states in which some sets are expanded, and a fully expanded state in which all sets are expanded.

The transmission system 7 may be implemented in any manner suitable for the specific implementation. The transmission system 7 may comprise a first transmission 8 for actuating movement of one or more movable parts 3a, 3b of a first set 3 of movable parts. The transmission system 7 may further comprise a second transmission 8″ for actuating movement of one or more movable parts 3c,3d of a second set 3′ of movable parts. A transmission can for example be a linear spreader or a scissor jack or other a linear transmission which can drive the movement of the movable parts away from, and optionally back towards, their first position in the non-expanded state, such as a rotary-to-linear transmission or hydraulic or pneumatic transmission. For example, the implantable device 1 can use a classic wedge transmission, a parallel bar and linkage transmission, a screw jack, a cam system, a balloon and bellows system, a longitudinal deformation/crush system (in which longitudinal contraction creates vertical expansion), or a stacking system, to name a few. A transmission 8,8′ may be operable independently from the other transmission to selectively drive a set of movable parts. For instance, as explained below in more detail, in the examples the operation of the first transmission 8 is decoupled from the operation of the second transmission 8′ and the first set 3 can be expanded separately and independently from the expansion of the second set 3′.

As explained in detail with reference to FIGS. 3-6, the transmission system 7 may extend between a tool interface 16, more specifically between first and second drive parts 16a, 16b, on the one side and the expandable structure 5 on the other side. On each the drive parts 16a, 16b a force may be exerted to move that drive part, e.g. a torque, compressive or tensile force. The transmission system 7 engages on the load resisting surface 6a,6b; 6c,6d to transfer at least a part of the force exerted on a respective drive part 16a,16b to the load resisting surface 6 via the first and second transmissions 8,8′ of transmission system 7. In doing this, the transmission system 7 actuates in the second example a displacement of selected load resisting surfaces 6a,6b; 6c,6d in the direction of expansion. More specifically, the movement of the drive part 16a, 16b causes a series of movements of the movable parts of the transmission system 7 which is transferred to one, or more but not all, of the sets of movable parts 3a-3d. In this example the movement is a displacement along the path controlled by the transmission system 7 of the movable part 3a-3d. For instance, the transmission system 7 can transfer the force exerted on the drive part relative to the proximal end 40 of the implant body into a force doing positive work in the direction of expansion d, which force acts on the movable part 3a-3d. The transmission system 7 can for example change the direction of the force, e.g. when the force on the drive part 16a, 16b is not in the direction of expansion and/or change the magnitude of the force exerted to a magnitude suitable to expand the implant against the loads acting thereon. The transmission system 7 can e.g. change the movement of the drive part 16a, 16b, rotational in this example, into a push-out of the movable part 3a-3d outwards from the implant body. In the example, the push-out pushes the respective movable part away from the lateral axis of the support structure, in a direction extending radially away from the lateral axis.

The movable parts 3a, 3b of a set 3 of movable parts may be form-closedly connected to each-other. In the example for instance, each movable part in a set is form-closedly connected to another movable part of the same set. As can be seen in FIG. 2C and D in more detail, the form-closedly connected movable parts of a set lie in the longitudinal direction of the implant at the same location and have their load resisting surface facing in the opposite direction. The connection is formed by respective ring-shaped members 300,301. In this example, each member 300,301 comprises a closed loops which extends through slots 302 in the connected movable parts. The loops are in this example rigid and not elastic, shape-retaining. The slots extend in the direction of expansion, and thus determine together with the closed loops the maximum extent to which the connected movable parts can be moved away from each other.

The first transmission 8 may comprise elements which are movable relative to each other to push the movable parts 3a,3b of the first 3 set outwards, in the direction of expansion along its path. In some implementations, the first transmission 8 may comprise one or more pairs of first elements 9,11, each pair of first elements comprising a first fixed element 9 and a first movable element 11. Referring to FIGS. 2B and 3C, in the shown example the first transmission 8 comprises a first fixed element 9, fixated in position relative to the support structure 2 and in this implementation an integral part thereof, and a first movable element 11 which is movable relative to the support structure 2. The movable parts 3a, 3b of the first set 3 may be arranged, seen in the longitudinal direction of the implant, between a pair of first elements, e.g. between the first fixed element 9 and the first movable element 11. The movable parts 3a, 3b may be at least partially supported by the first fixed element 9 and the first movable element 11. In a non-expanded position of movable part 3a, 3b, the load resisting surface 6a, 6b of the movable part 3a, 3b is at a first location at a distance from the elongate shaft 10.

The first fixed element 9 may be located at a first position and the first movable element 11 may be located at a second position distanced from the first fixed element 9 by a first distance d1, with the distance d1 being adaptable by moving the first movable element 11. As the first movable element 11 is moved towards the first fixed element 9, this pushes the movable part 3a, 3b to be displaced in a direction perpendicular, or substantially perpendicular, to the longitudinal axis of the elongate shaft 10, e.g. the direction of expansion. This will be described in more detail with reference to FIGS. 4A-C. As can best be seen in FIGS. 3C and 4C, the first side 18 of the base 17 of the movable parts may touch the first fixed element 9 and the second side 19, may touch the first movable element 11. By moving the elements 9,11 towards or away from each other, their inclined surfaces slide over the base 17 and the sides 18,19 are pushed outwards in the direction of expansion. In some implementations, such as those in which the base is hollow or substantially hollow, the first side 18 and the second side 19 may comprise edges of the frame.

Each of the first fixed element 9 and the first movable element 11 may comprise an inclined surface. Each of the first fixed element 9 and the first movable element 11 may comprise for instance a conic region, such as a cone or a truncated cone For example, the first fixed element 9 may comprise one or more fixed wedges, and/or the first movable element 11 may comprise one or more movable wedges. The inclined surface of the first fixed element 9 may e.g. face towards or away from the inclined surface of the first movable element 11. In some implementations, such as those described above with reference to FIGS. 2C and 2D, the incline of each of the first side 18 and the second side 19 of the base 17 may be arranged to correspond to an incline of the first fixed element 9 and an incline of the first movable element 11, respectively.

The first transmission 8 can be implemented in any manner suitable to change the distance between the first fixed element 9 and the first movable element 11. In the second example, the first movable element 11 is mounted on an elongate shaft 10 which extends through the support structure 2. The shaft 10 can be fixated in position in the longitudinal direction of the support structure 2, for instance. In this example, the movable element 11 has a longitudinal, threaded, through-hole through which a threaded region 110 of the shaft 10 extends and which engages with the thread of the hole. Said differently, in this example the first movable element 11 and the shaft 10 form a moving nut-spindle system of which the shaft 10 acts as a power screw and the first movable element 11 engaging with the treaded region of the shaft 10 forms a moving nut, which is movable along the power screw, that is shaft 10, in the axial direction of the power screw. The movable element 11 is translationally movable along the shaft 10 by rotating the shaft 10 around its longitudinal axis. In this, the support structure 2 inhibits rotating of the movable element 11 around the axis of the power screw and thus ensures that movable element only moves translationally along the axis of the power screw, parallel to the longitudinal direction.

The threaded connection between the elongate shaft 10 and the first movable element 11 provides mechanical stability and reliability to the implant 1 and may prevent unintentional motion of the first movable element 11. For example, once the operator (e.g. surgeon) has rotated the elongate shaft 10 until the load resisting surface 6a, 6b of the first movable parts 3a, 3b of the first set 3 are in a desired position (e.g., until the first movable element 11 is in a corresponding position along the elongate shaft 10), the threading on the elongate shaft 10 and the first movable element 11 may prevent the first movable element 11 from moving forward or backward along the elongate shaft 10 when the elongate shaft 10 is not rotated. Thereby the implant 1 is maintained in the elected expanded state to the degree of expansion, even when the load acting on the movable parts is transferred onto the transmission 8.

The first fixed element 9 and/or the first movable element 11 may be concentrically arranged around the elongate shaft 10. In the second example, the elements 9,11 are shaped as cones or truncated cones which share their axis with the axis of the elongate shaft 10. The default position, that is in the non-expanded state, of the first movable element 11 may be a position in which the first movable element 11 is maximally distanced from the first fixed element 9.

The second transmission 8′ may also comprise elements which are movable relative to each other to push movable part(s) 3c,3d of the second set 3′, on which the second transmission acts, outwards in the direction of expansion along the respective path of the movable part. The second transmission 8′ may comprise a second fixed element 12, which is fixated in position relative to the support structure 2, and a second movable element 14 which is translationally movable relative to the support structure 2. The second movable element 14 and/or the second fixed element 12 may be shaped similarly to the first movable element 11 and/or the first fixed element 9 of the first transmission 8 and be movable to expand the second set in a manner similar to the pair of elements 9,11 of the first transmission 8.

The second fixed element 12 may be positioned at a third location along the elongate shaft 10. The second movable element 14 may have a default position, that is in the non-expanded state, at a fourth location along the elongate shaft 10. In some implementations, the second fixed element 12 may be located at a location, seen from the distal end 41 in the longitudinal direction of the implant, further away from the first fixed element 9 of the first transmission 8 than the first movable element 11 of the first transmission 8. The third location may be fixated relative to the elongate shaft 10. In the shown implementations, the elongate shaft 10 extends through a passage in the second fixed element 12, and is freely rotatable around its longitudinal axis in the passage without engaging on the second fixed element 12.

The second transmission 8′ can be implemented in any manner suitable to change the distance between the second fixed element 12 and the second movable element 14. In some implementations, the second movable element 14 may be a part, or region of a telescopic actuator, and the transmission 8′ be implemented as a telescope actuator which extends through the implant body 1 from the proximal end 40 to the location of the second set 3′. In the examples, the second movable element 14 is the end of a tubular element 13. The tubular element 13 is slideably mounted in a bore in the implant body 1, in this example the element 13 is slideably mounted in the tubular inside of the support structure 2, to telescopically project out of the bore. The element 13 has an outer shape conforming to the interior shape of the bore. In this example, the element has in cross-section perpendicular to the sliding direction, a round outer shape but alternatively this may be elliptical, square or rectangular. The bore extends in this example in the longitudinal direction of the implant. In the examples, the tubular element 13 extends inside the bore from an end of the bore at which the movable element 14 is located towards the opposite end of the bore, at proximal end 40, up to a location at a distance from the opposite end.

Actuation, e.g. rotation, of a second drive part 16b relative to the support structure 2 causes the tubular element 13 to slide telescopically inside the bore and project more or less out of the bore. As the tubular element 13 is moved longitudinally, the second movable element 14 moves towards the second fixed element 12 and the increasing proximity of the inclined surfaces of the second fixed element 12 and the second movable element 14 causes the movable part 3c, 3d to be displaced in a direction perpendicular, or substantially perpendicular, to the longitudinal axis of the elongate shaft 10, e.g. the direction of expansion. This will be described in more detail with reference to FIGS. 5A-C.

In this example, the second drive part 16b is arranged such that a rotational movement thereof around the longitudinal axis of the bore translates into a translational movement in the direction of that axis. Although various alternative implementations are possible, in this example the second drive part 16b extends inside the bore between the opposite end of the bore, at the proximal end 40 of the implant body 1, and the tubular element 13. The second drive part 16b can be driven to move in the longitudinal direction of the bore and to push against the tubular element 13. Although the second drive part may be driven in another manner, in this example as the second drive part 16b is rotated in the bore with respect to the support structure 2, the second drive part 16b moves longitudinally. The second drive part 16b has an outer shape conforming to the interior shape of the bore. In this example, the element has in cross-section perpendicular to the sliding direction, a round outer shape which allows the drive part to rotate in the bore. However, for instance in case of a non-conforming shape the second drive part may e.g. have an elliptical shape instead.

Said differently, the tubular element 13 and the second drive part 16b can be regarded as a segmented cylinder, segmented in cylinders in the bore, with a slideable but not rotatable cylinder formed by the tubular element 13 and a driving cylinder formed by the second drive part 16b which is rotatable but not freely slidable. The driving cylinder is rotatable in the bore to translationally move in the bore. The second drive part 16b comprises a region which engages with the wall of the bore to transfer the rotating in the translational movement and thereby push the slideable section to slide in the bore. For example, said region may be threaded and engage with a corresponding threaded section of the wall of the bore, such that rotation of the second drive part 16b with respect to the support structure 2 effects longitudinal movement of the second drive part 16b.

In this example, the second drive part 16b is a hollow element and the elongate shaft extends from the proximal end 41 towards the distal end 41 through the second drive part 16b and the tubular element 13 to project out of the tubular element, at the location of the second movable part 14. The tubular element 13 and the second drive part 16b are slidable over the elongate shaft 10, and in this example do not engage thereon. Thus, a rotational movement of the shaft 10 is not transferred on the second transmission 8′ and vice versa, rotational and/or translational movements of the tubular element 13 and the second drive part 16b are not transferred to the first transmission 8. The operation of the first transmission is therefore decoupled from the operation of the second transmission. The position of movable parts of different sets, i.e. at different longitudinal positions along the device 1 can therefore be adjusted independently, such that a movable part 3a of a first set 3 may be expanded to a different extent than movable part 3c of another set 3′.

One or more, preferably all of the elongate shaft 10, the tubular element 13, the second drive part 16b, and the bore in the supporting structure 2 may have a parallel axis, e.g. may be coaxial. This allows a relatively simple construction of the transmission system 7. For instance, the second drive part 16b and the elongate shaft 10 may be rotatable around the same axis, which may be the longitudinal axis of the bore in the support structure 2. Also, the tubular element 13 and the second drive part 16b may be translationally movable in the direction parallel to the longitudinal axis of the bore in the support structure 2. However, more complex transmission systems are also possible, for example with two radially offset power screws that engage via a gear box on the respective movable parts, for instance.

The device 1 may further comprise a tool interface 16, also referred to as a drive part 16, for a surgical tool. The tool interface 16 may enable a tool, such as a surgical tool, to engage with the transmission to actuate the movement of the movable parts with the tool. The implant can thus be brought from the non-expanded state into an expanded state by using the surgical tool, engaged with the tool interface 16, to drive the transmission system 7 and actuate a movement of one or more movable parts 3a-3d in one or more of the sets 3,3′ of movable blocks the implant 1. In the second example, the tool interface 16 enables the tool to selectively engage with the first transmission 8 and/or the second transmission 8′ of the transmission system 7. To that end, the tool interface 16 comprises a first drive part 16a for a first transmission 8 of transmission system 7, and the second drive part 16b for a second transmission 8′ of transmission system 7. This will be described in more detail with reference to FIGS. 7-10. The surgical tool can be any type of drive tool compatible with the interface which, when engaged with the interface 16, drives the transmission system 7. Various drive tools are known in the art. The surgical tool may be a manually, hydraulically, pneumatically, electrically, electro-magnetically, magnetically or mechanically driven tool, and/or may use a suitable type of mechanical transducer to drive the movement. The tool may e.g. be disposable. The surgical tool may be a medically acceptable, e.g. sterile and biocompatible, tool. An example of a suitable tool will be described in further detail with reference to FIGS. 7-10.

The tool interface 16 can be arranged to drive expansion of the expandable structure at a selected point in time, in the second example after anchoring the implant 1 in the bone. For instance, in the implementations, each drive part 16a,16b drives a different set of movable parts, i.e. by engaging on a different transmission 8,8′. In some implementations, tool interface 16 may be located at, or on, an end of the support structure 2. The tool interface 16 is in the second example located at the proximal end 40 of the anchor body 4 but this may be at another location of the implant accessible during surgery after positioning the implant 1 in the cavity.

While placing the implant, the movable parts 3a-3d and the load resisting surfaces 6a-6d are positioned and oriented as deemed suitable in the cavity by a medical practitioner. Thus, prior to expansion, the implant 1 is already in position and oriented, with the expandable structure 5 in the non-expanded state oriented and positioned to support the bone after expansion as deemed suitable by the medical practitioner. The drive parts 16a, 16b of the tool interface 16 are movable relative to the proximal end 40 of the implant This movement of the respective drive part 16a, 16b of the tool interface 16 drives an expansion of the expandable structure 5, in this example a movement of one or more of the movable parts 3a-3d along its predetermined path. In some implementations, a drive part can be rotated relative to the support structure 2, in these implementations relative to the proximal end 40. The use of rotational motion to drive the expansion of one or more movable parts may reduce or avoid further damage to the bone during placement but the drive part may alternatively be implemented to be pushed towards or pulled away from the support structure for instance.

As mentioned before, the implant 1 may comprise a bone anchor. Referring to FIG. 2A, the implant may in some implementations comprise an anchor body 4 with a distal end 41 and a proximal end 40, at a distance from the distal end 41. Here, the term “distal” is used in the sense that after implantation the distal end 41 lies the deepest into the bone, and the proximal end 40 is then closer to, preferably flush with, or projecting outwards from, the bone surface. Although the implant can be implemented to be anchored into cancellous bone, in the following implementations are described in which the implant is constructed to be anchored into cortical bone. In this example, the implant 1 is a vertebral body implant which can be anchored into the pedicle, with the expandable structure 5 of the implant located in a cavity in the vertebral body, to provide support to the vertebral body against external loads acting on the vertebrae. The support may be provided in a direction parallel to the cranial-caudal plane and the loads may be acting on the vertebral end-plates, for instance.

The anchor body 4 serves to anchor the implant 1 to or in a part of a bone, e.g. in the pedicle 107. When anchored, the anchor body 4 may resist forces in the distal-proximal direction of the anchor body 4. The anchor body 4 then inhibits or blocks translational movements of the implant 1 relative to the bone part to which the anchor body is anchored, at least in the distal-proximal direction, and, optionally, inhibits or blocks rotational movements around an axis extending in the distal-proximal direction as well. In addition, the bone part may surround the anchor body 4 such that translational forces perpendicular to the distal-proximal direction or rotational forces around an axis perpendicular to the distal-proximal direction are resisted as well. The expandable structure 5 has in this example no degrees of freedom relative to the anchor body 4 and the expandable structure 5 is unmovable relative to the anchor body 4. A movement of the anchor body 4 thus moves the expandable structure 5 and a movement of the anchor body 4 leads to the same movement of the expandable structure 5, which provides a direct handling and facilitates positioning and orientation by the medical practitioner. As elucidated hereinafter in more detail, the expandable structure 5 is fixated to the anchor body 4, more specifically to the distal end 41. This allows a precise positioning and orientation of the expandable structure 5 in the cavity.

In this example, a movement of the tool interface 16 relative to the support structure 2 does not result in a change in position or orientation of the expandable structure 5 relative to the anchor body 4. It will be apparent though, that adjustments may be made in the position or orientation of the expandable structure 5 after (partial) expansion, e.g. by reducing the expansion and then manipulating the implant 1 to e.g. extend deeper or less deep into the bone and/or to rotate the implant 1 around a longitudinal axis to change the orientation of the expandable structure 5.

Typical dimensions of the implant 1 with a bone anchor (although other sizes being possible as well depending on the cavity in which the device is to be placed) can be a length between 25 mm to 65 mm. For example, the length can be 25 mm or more, such as 35 mm or more. The length can be less than 65 mm, for example less than 60 mm, for instance 40 mm or less. A currently preferred range for the length is a length between 40 and 60 mm. A typical maximum, non-expanded diameter (that is the dimension in the direction of expansion, in this example perpendicular to the longitudinal direction of the implant) can for example be less than 10 mm, such as less than 8 mm, such as 5 mm or less. Preferably, but not necessarily, that diameter is 1 mm or more, such as 1.5 mm or more. 2.25 mm or more, such as 3 mm or more. A typical maximum expansion of the device is for example between 1.5 and 4 times the non-expanded diameter. Other maximum expansions are likewise possible, and it is currently preferred that the maximum expansion is less than 5 times the non-expanded diameter which ensures a mechanical stable and reliable expansion.

The anchor body 4 may be implemented in any manner suitable for the specific implementation. In the shown example, the anchor body 4 is a monolithic body made in one piece, but alternatively it may be composed of several separate pieces which are e.g. screwed onto each other. As elucidated below in more detail, in a currently preferred example, the anchor body is a non-porous structure, but alternatively the anchor body may have a porous outside and/or partially or completely porous inside. For example, the anchor body may be provided with through pores from the outside to the bore extending inside.

The anchor body 4 may have any suitable shape. The anchor body 4, can, for example, have a smooth shape, i.e. the cross-section may be constant or vary, monotonically or not, (e.g. tapers) along the longitudinal direction, either locally or over the whole length. For example, the distal end 41 may be tapered whereas from the proximal end to the location of the expandable structure 5 the cross-section may be constant. In this example, though the diameter is constant over the whole length.

The anchor body 4 may be an elongate body, e.g. rounded or not rounded. In this example, the anchor body 4 has for example rounded shape, more specifically a cylindrical shape, and although in this example this is a circular cylindrical shape, other cylindrical shapes such as elliptical cylinders may also be suitable, as well as other rounded shapes such as a cuboid (or other polyhedrons) with chamfered or fillet lateral edges, for instance. The anchor body 4 may, as in this example have a longitudinal axis a parallel to the longitudinal direction I of the implant, around which one or more of the drive part 16a, 16b may be rotatable relative to the anchor body 4, for example, as indicated in the FIGs with the arrow.

The anchor body 4 can be provided with a bore, such as a cannula, extending from the proximal end 40 towards the distal end 41. In such a case the transmission system 7 can extend through the bore, and thus be embedded inside the anchor body 4. This reduces the risk that e.g. during insertion in the bone the transmission system 7 is damaged or gets stuck, such as due to bone fragments or chips getting stuck between the transmission system 7 and the anchor body. Although the bore can be provided in differently shaped bodies, in this example the anchor body 4 has a tubular shape, and the bore has an open end at at least one, and preferably a both, of a side facing the proximal end 40 and a side facing the distal end 41, through which the transmission system 7 projects. The bore is the bore in the supporting structure 2 described before through which the first and second transmission 8,8′ extend. The bore extends in this example from the proximal end up 40 up to the location of the expandable structure 5. In this example, at that location the second movable element 14 is located in the non-expanded state of the implant.

The outside of the anchor body 4 can have a friction enhancing profile for holding the implant 1 in the part of the bone 10. The anchor body 4 can for example, as indicated in FIG. 2A, have an outer surface 48 extending in the lateral direction, which may be unprofiled, partially profiled or completely profiled. The profile may for instance be ribbed, fluted and/or provided with helical threads. In this example, the outer surface 48 has a profiled area 48a where the outer surface is provided with a profile that extends circumferentially around the anchor body 4 and extends in the lateral direction from the proximal end 40 up to an unprofiled area 48b, the unprofiled area 48b extending extends up to the distal end 41. In this example, the expandable structure 5 is located in the unprofiled areas 48b. Thus, the anchoring force is not exerted on the bone in the area of the expandable structure 5, where the bone will typically be relatively weak and hence susceptible to further damage. Accordingly, despite being a single mechanical system still a spatial separation of the forces exerted on the bone can be obtained.

In this example, the outside of the anchor body has an elongate shape which is provided (in the profiled area 48a) with ridges extending at an angle relative to the longitudinal direction. Although the ridges may e.g. all parallel (each ridge forming a closed loop), in this example the ridges are connected and form a helical thread 49. The tread 49 may be sufficiently sharp and rigid that upon rotational insertion in a pre-drilled cannula in the bone, the anchor body forms a thread in the cannula, complementary to the thread 49 of the body, and the anchor body 4 may thus be a thread forming screw body. In the shown example, this screw body is not self-tapping and accordingly is inserted in a pre-drilled cannula 104, as illustrated in FIG. 12-14. However, alternatively the anchor body 4 may be self-tapping and e.g. at the distal end 41 be provided with a sharp point, and along the outside surface be provided with a self-tapping thread which extends from the point towards the proximal end 40.

The expandable structure 5 is in the second example attached relative to the anchor body 4 as follows. The part of the anchor body 4 in which the expandable structure 5 is located may, as in the example, be shaped as a slotted tube. In the initial, that is non-expanded state of the implant, a movable part 3a-3d is admitted e.g. at least partly, in this example complete recessed in the slotted tube. The load resisting surface 6a,6b; 6c,6d may then for example be flush with, or below, the outer surface of the anchor body 4. As can best be seen in FIG. 2C, for example the distance between the longitudinal axis and the load resisting surface 6a,6b; 6c,6d may in the radial direction be less than the diameter of the implant body, in this example the tubular body formed by the anchor body 4. Alternatively, the distance may be equal or larger than the diameter. For instance, the movable part 3a-3d may project in the radial direction (preferably slightly) beyond the outer surface.

In this example, a space 42 in which the expandable structure is located is formed by a slot of the slotted tube. The slot has an opening 43 extending parallel to the longitudinal direction. A respective movable part 3a-3d is movable through the opening 43 to the expanded position, as can be seen in FIG. 4. The slotted tube has in this example two, opposite openings 43 facing each other, such that the anchor body has a fork-shaped distal end 41 with prongs 44 extending in the longitudinal direction. As shown, the space 42 is formed between the prongs 44, and the expandable structure 5 is located therein. In this example, at each expansion side the opening 43 is large enough to allow the movable parts of both sets 3,3′ to pass through the same opening. As can be seen in FIG. 2B for instance, the length of the opening is as large, or larger than two movable parts. In an alternative implementation for instance, each movable part has its own opening which is separated from the movable parts by e.g. a closure formed in the tubular body.

The prongs 44 are at one end thereof attached to each other by the anchor body 4, and at the other end by a cap 45 which forms the tip of the implant and thus the distal end 41 in this example. The cap 45 may be implemented in any manner suitable for the specific implementation. The cap 45 may be cone-shaped, as in this example. The cap 45 and the prongs can be a single piece, such as integrally formed together or joined together after forming. Alternatively, as in this example, the cap 45 may be a separate piece fixated to the prongs 44. In this example, the prongs 44 are joined at the tip side end by a bridging end part of the anchor body which bridges the ends of the prongs and on which the cap 45 can be mounted. As can be seen in FIGS. 3C,4C,5C and 6C for instance, the end part may for example be provided with a threaded bore in which the screw part of a screw cap can be screwed. Alternatively, for example, the screw cap can be welded or otherwise jointed to the anchor body 4. As more clearly shown in FIG. 6, for example, the screw cap 45 may comprise a hole 46, in this example a blind hole which closes of the distal end 41, and in which a terminating part of the transmission system 7 can be mounted, e.g. the distal end of the elongate shaft 10 in this example. The screw cap 45 further comprises a cone-shaped part 47 which is oriented with the apex towards the distal end 41, thus forming a pointed tip of the implant.

Although in this example the expandable structure 5 is thus located in the anchor body 4, at the proximal side of the distal end 41, alternatively the expandable structure 5 may be located between the distal end 41 of the anchor body 4 and the tip of the implant 1. In such a case, the prongs 44 may for example be implemented as an integral part of the cap and e.g. be screwed or otherwise attached to the anchor body 4.

The implant may be provided in an assembled state. Alternatively, a kit of parts for assembling an implant 1 may be provided. Such a kit may, as illustrated in FIG. 2B, comprise the anchor body 4, (if present) the components of the expandable structure 5 (either already assembled or as separate parts), the transmission system 7 and, optionally other parts. The shown example may be assembled by first positioning the expandable structure 5 as well as the transmission system 7 in the space 42, with these elements aligned on the axis a of the anchor body 4. Subsequently, the elongate shaft 10 may be inserted from the distal end 41, such that it passes through the space 42 and respective ring-shaped links that link the elongate shaft 10 to the movable parts 3a-3d, into the bore. The cap 45 may then be placed on the distal end 41 to secure the elongate shaft 10 relative to the anchor body 4. The expandable structure 5 is put in the non-expanded state by moving the transmission system 7 in the corresponding position and pushing the movable parts 3a-3d towards the axis, for example until they are maximally interdigitated. Prior to this, or thereafter, the load resisting surfaces 6a-6d may be mounted as elucidated earlier

The kit or the implant 1 can be provided in a, preferably sterile, package, either alone, together with other components of an implant system (such as a pharmaceutical formulation to be applied, bone cement compositions, a surgical toolset, or otherwise) and/or with other medical devices. The package may be labelled or provided together with instructions to use the implant 1 in a type surgery, and/or for the treatment of a condition, selected from the group consisting of: vertebral fracture, collapse of vertebral end-plates, vertebral height restoration, trauma fracture, or in-vivo implantation in at least one selected from the group consisting of: non-human animal, human, domestic animal, pets, livestock. Other examples may be: internal skeletal fixation, external skeletal fixation, posterior fixation, in combination with pedicle screws and rods system, Lumbar interbody fusion (LIF), Anterior LIF (ALIF), Transforaminal LIF (TLIF), Lateral LIF (LLIF), Posterior LIF (PLIF). Examples of such conditions are: Degenerative disc disease, Spondylolisthesis, Spinal stenosis, Scoliosis, Spinal disc herniation, Discogenic pain, Spinal tumor, Kyphosis, Lordosis. The label may for example indicate use in a patient with for example a spinal condition such as a vertebral tumor.

Also, the label may for example indicate use in a patient a thoracolumbar or other spinal fracture. The spinal fracture may e.g. fall one or more categories in the group consisting of: compression injuries, distraction injuries, torsion injuries. More specifically, the patient may have a compression injury which falls one or more categories in the group consisting of: impaction fractures, endplate impaction, wedge impaction, vertebral body collapse, split fractures, frontal split fracture, sagittal split fracture, pincer fracture, burst fractures, incomplete burst fracture, burst split fracture, complete burst fracture, pincer, flexion, axial. Additionally or alternatively, the patient may have a distraction injuries which falls one or more categories in the group consisting of: predominantly transligamentous flexion-distraction injury, predominantly osseous flexion-distraction injury, anterior disruption through the disc. The predominantly transligamentous flexion-distraction injury may for example be with a transverse disc disruption (with or without a flexion subluxation and/or an anterior dislocation and/or with or without fractures of the articular processes, with type A vertebral body fracture. The predominantly osseous flexion-distraction injury may for example be a transverse bi-column fracture, a posterior osseous disruption with transverse disc disruption (e.g. through the pedicles and/or through the interarticular portions (flexion spondylolysis)), with type A vertebral body fracture (e.g. through the pedicles or through the isthmus), anterior disruption through the disc (with or without hyperextension-subluxation, hyperextension-spondylolysis) and/or a posterior dislocation. C: Also, the patient may have a torsion injury which falls one or more categories in the group consisting of rotation-compression injury, rotation-distraction injury, rotational shear injury. A rotation-compression injury may for example be of the impaction type, the split type or the burst type. A rotation-distraction injury may e.g. be with transligamentous flexion-distraction and/or with trans-osseous flexion-distraction and/or with hyperextension-distraction.

Referring now to FIGS. 3-6, although other implementations may likewise have such states, various states of the implant are elucidated using the implementation of an implant of FIG. 2 as an example.

FIGS. 3A-C show the implant in a non-expanded state. The non-expanded state may refer to a state of device 1 in which all of the movable parts 3a-3d are at the location on the path closest to the centre of the implant 1. The dimensions of the implant may be at their minimum when the device 1 is in a non-expanded state, e.g. when none of the movable parts 3a-3d are expanded. For example, the implant may be expandable in a direction perpendicular to the direction in which the implant is moved into the bone, and e.g. the thickness, width or diameter of the device 1 may be at its respective minimum. In some implementations, the load resisting surfaces 6a-6d may in the non-expanded state be recessed within the support structure 2 or be flush (or substantially flush) with an outer surface of the support structure 2. When the movable part 3a, 3b is in its default position, that is in the position of the non-expanded state, for example base 17 may be retracted, or mostly retracted, and admitted into the support structure 2. As illustrated in FIG. 3B, the implant may comprise multiple expansion sides at which the implant 1 is expandable in a respective direction of expansion, here in a radial direction away from a longitudinal axis of the support structure 2. For example, the movable parts 3a, 3b of the sets 3,3′ at the first expansion side may be movable in a first direction of expansion away from the expansion side, whilst the movable parts 3b, 3d of the sets 3, 3′ at the second expansion side may be movable in a second direction of expansion away from the second expansion side, the first direction of expansion being opposite or substantially opposite to the second direction of expansion.

As can be seen in FIG. 3C, in the non-expanded state, the transmission system 7 is its initial setting, with the first transmission 8 and the second transmission 8′ in their initial position. When the transmissions 8,8′ are in the initial position, he first movable element 11 and the second movable element 14 are in their respective initial positions. The movable parts 3a-3d are then at an initial location, and the device 1 may have a minimal thickness (in case of a tubular device body this may also be referred to as the diameter of the device 1) in the direction of expansion. As shown, here in the non-expanded state, movable parts 3a-3d are recessed in the implant body 1.

In one or more expanded states, the movable parts 3a-3d may be transferred, from their initial location in the non-expanded state of the implant, to a second position in which the movable part projects relative to the shape of the implant in the non-expanded state. In an expanded state, the expanded shape of the implant 1 projects compared to the non-expanded shape of the implant in one or more directions of expansion. The device 1 may for example have an expanded thickness, larger than the minimal thickness, equal to or less than the maximum expanded thickness. In the shown example, the first set 3 of movable parts 3a,3b may be moved, independent from the second set 3′ of movable parts 3c, 3d, by actuating the first transmission 8, such as by a surgical tool engaging on the drive part 16a of the first transmission 8. This will be described in further detail with reference to FIG. 4. The second set 3′ of movable parts 3c,3d may be moved, independent from the first set 3 of movable parts, by actuating the second transmission 8′, such as by a surgical tool engaging on the drive part 16b of the second transmission 8′. This will be described in further detail with reference to FIG. 5. By simultaneously or sequentially actuating the first transmission 8 and the second transmission 8′, the implant 1 may be brought into a fully expanded state, also referred to as a maximally expanded state. This will be described in further detail with reference to FIG. 6. In these figures, movements of the respective parts are indicated with arrows.

FIGS. 4A-C show an expandable intra-osseous implant in a first expanded state. To facilitate understanding of the operation, in the shown first expanded state only the first set 3 of movable parts 3a, 3b is expanded, while the other sets of movable parts are shown in their default position. For conciseness, only the transmission and configuration relating to the first set 3 of movable parts 3a, 3b are explained, although it will be readily appreciated that the transmissions and configurations described herein will apply also to an expanded state in which multiple (e.g. both) sets 3,3′ of movable parts 3a-3d are expanded by the same transmission, such as the third expanded state illustrated in FIG. 6. Furthermore, the first set 3 of movable parts 3a, 3b is shown expanded to the maximal extent but may be expanded to any extent between the non-expanded state and the maximal extent. As described above, the first set 3 of movable parts 3a, 3b may be expanded within a continuous range between its initial position in a completely non-expanded state to a maximally expanded position. Although FIG. 4A illustrates a set 3 with a single pair of movable parts, the movable parts of each pair being opposite to each other and parallel to all other sets, it is to be understood that, more than two movable parts may be used. For example, there may be two or more pairs of movable parts. In other implementations, the set may for instance have a single movable part which is located with a longitudinal or transverse offset at the same expansion side as a movable part of another set.

As illustrated in FIG. 4B, in the first expanded state the first set 3 of movable parts has been expanded. The movable parts 3a, 3b have been moved in respective expansion directions away from the expansion side. The respective expansion directions may be perpendicular to a longitudinal axis of the device 1, e.g. in this example corresponding to the longitudinal axis of the elongate shaft 10. In this example, in the first expanded state, the load resisting surface 6a, 6b of movable part 3a, 3b protrudes, in the direction of expansion from the support structure 2. As the movable part 3a, 3b is expanded, base 17 has emerged, e.g. protrudes, from the support structure 2, in this example in the radial direction, as can be seen in FIG. 4B. Although a movable part 3a,3b may be expanded to remain partially recessed, in this example the movable parts are moved completely out of the space 42 in the implant body.

In this example, movement of movable parts 3a, 3b may be achieved through the use of the first transmission 8 of the transmission system 7. Referring now to the cross-sectional view of device 1 in FIG. 4C, in the first expanded state, the distance between elements 9,11 is changed, in this example reduced. The first movable element 11 has been moved from a first location shown in FIG. 3C to a second location as shown in FIG. 4C. The first movable element 11 has been brought as close as possible to the first fixed element 9, such as to abut against the first fixed element 9. It is to be understood, however, that the first movable element 11 may be moved to any intermediary location from the continuous range between the first location and the abutting location.

As the inclined surface of the first movable element 11 approaches the inclined surface of the first fixed element 9, e.g. as the distance between the first movable element 11 and the first fixed element 9 decreases, the load resisting surface 6a, 6b may be expanded in the direction of expansion. In this example, as the first movable element 11 moves towards first fixed element 9, the first movable element 11 applies a force against the base 17a, 17b of the movable part 3a,3b, causing a displacement of the load resisting surface 6a,6b in the direction of expansion (e.g. perpendicular to the longitudinal axis of the elongate shaft 10). The displacement of the load resisting surface 6a,6b may thus be adjusted to any desired location within the continuous range between a non-expanded position of the load resisting surface 6a,6b and a maximally expanded position of the load resisting surface 6a,6b, corresponding to the continuous range of the displacement of the first movable element 11 from its default position (e.g. first location as shown in FIG. 3C) to its maximally displaced position (e.g. second location as shown in FIG. 4C). When the first movable element 11 has been maximally displaced from its default (e.g. first) location, e.g. when the first movable element 11 is as close as possible to the first fixed element 9, the load resisting surface 6a of movable part 3a is maximally expanded in the direction of expansion.

The first movable element 11 may be moved longitudinally towards the first fixed element 9, for instance along the elongate shaft 10 by rotating the elongate shaft 10 within the device 1 around its longitudinal axis. In this example, the threaded surface of the first movable element 11 may engage with the threaded region 110 of the elongate shaft 10. The elongate shaft 10 may rotated, for example by using a tool such as a surgical tool. For example, the tool interface 16 may comprise an engageable head of the elongate shaft 10, such as a head of a screw or other drive part 16a allowing to drive the movement of the elongate shaft 10 with a surgical tool engaged on the drive part 16a. The engageable head of the elongate shaft 10 may be shaped to engage with the surgical tool. For example, the head may be a cross-shaped (e.g. a Phillips-head), slot-shaped (such as a flat-head or slot drive), a hexagonal shape, star-shaped (e.g. a Torx head or hexalobular socket drive), a double hexagonal shape, or square-shaped (e.g. a Robertson head). As can be seen in FIG. 4C, in this example relative to the support structure 2 the elongate shaft has only rotated around its rotational axis and not been moved in the longitudinal direction. Equally, the fixated element 9 has not moved in the longitudinal direction. Furthermore, the second transmission 8′ has not been operated by the rotation of the elongate shaft 10.

It will be apparent that as in alternative implementation, wedges may be used which face away from each other, and increasing the distance between them may result in expansion in the direction of expansion.

FIG. 5 shows an expandable intra-osseous implant in a second expanded state. The second expanded state is similar to the first expanded state and differs in the following. In the shown second expanded state only the second set 3′ of movable parts 3c, 3d is expanded, while the other sets of movable parts are shown in their default position. For conciseness, only the transmission and configuration relating to the second set 3′ of movable parts 3c, 3d are explained, although it will be readily appreciated that the transmissions and configurations described herein will apply also to an expanded state in which multiple (e.g. both) sets 3,3′ of movable parts 3a-3d are expanded by the same transmission, such as the third expanded state illustrated in FIG. 6. Furthermore, the second set 3′ of movable parts 3c, 3d is shown expanded to the maximal extent but may be expanded to any extent between the non-expanded state and the maximal extent. As described above, the second set 3′ of movable parts 3c, 3d may be expanded within a continuous range between an initial position in a completely non-expanded state to a maximally expanded position. Although FIG. 5A illustrates a set 3′ with a single pair of movable parts 3c,3d, the movable parts of each pair being opposite to each other and parallel to all other sets, it is to be understood that, more than two movable parts may be used. For example, there may be two or more pairs of movable parts. In other implementations, the set may have a single movable part and located with a longitudinal or transverse offset on the same expansion side as a movable part of another set for instance.

As illustrated in FIG. 5B, the second movable parts 3c, 3d have been expanded in respective expansion directions away from their respective expansion sides 5,5′. In this example, the expansion sides 5,5′ are the same for the first and second expanded state. The directions of expansion of the second set are parallel to the directions of expansion of the first set 3 illustrated in FIG. 4. The respective expansion directions may be perpendicular to a longitudinal axis of the device 1, e.g. the longitudinal axis of the elongate shaft 10. When the movable part 3c, 3d is in its default, e.g. non-expanded, state, base 17 of movable part 3c, 3d may be retracted or mostly retracted into the support structure 2. As the movable part 3c, 3d is expanded, e.g. by movement of the second movable element 14 of the transmission towards the second fixed element 12, the base 17 may emerge, e.g. protrude, from the support structure 2. Although a movable part 3c,3d may be expanded to remain partially recessed, in this example the movable parts are moved completely out of the space 42 in the implant body.

To put the implant in the second expanded state, the transmission system 7 has been used to actuate movement of the set of movable parts 3c and 3d. More specifically, the second transmission 8′ has been actuated to drive the expansion of the second set 3′. Referring now to the cross-sectional view of device 1 in FIG. 5B, the transmission system 7 may be configured to actuate movement of movable parts 3c, 3d as described above with reference to FIG. 2. That is, movement of movable parts 3c, 3d may be achieved through the use of the second transmission 8′ of the transmission system 7.

In this example, in the second expanded state, the distance between second elements 12,14 is changed, in this example reduced. The second movable element 14 has been moved from a first location shown in FIG. 3C to a second location as shown in FIG. 5C. The second movable element 14 has been brought as close as possible to the second fixed element 12, such as to abut against the second fixed element 12. It is to be understood, however, that the second movable element 14 may be moved to any intermediary location from the continuous range between the first location and the abutting location.

As the inclined surface of the second movable element 14 approaches the inclined surface of the second fixed element 12, e.g. as the distance between the second movable element 14 and the second fixed element 12 decreases, the load resisting surface 6c, 6d may be expanded in the direction of expansion. In this example, as the second movable element 14 moves towards the second fixed element 12, the second movable element 14 applies a force against the base 17 of the movable part 3c,3d, causing a displacement of the load resisting surface 6c,6d in the direction of expansion (e.g. perpendicular to the longitudinal axis of the elongate shaft 10). The displacement of the load resisting surface 6c, 6d may thus be adjusted to any desired location within the continuous range between a non-expanded position of the load resisting surface 6c, 6d and a maximally expanded position of the load resisting surface 6c, 6d corresponding to the continuous range of the displacement of the second movable element 14 from its default position (e.g. first location as shown in FIG. 3C) to its maximally displaced position (e.g. second location as shown in FIG. 5C). When the second movable element 14 has been maximally displaced from its default (e.g. first) location, e.g. when the second movable element 14 is as close as possible to the second fixed element 12, the load resisting surface 6a of movable part 3a is maximally expanded in the direction of expansion.

The second movable element 14 may be moved longitudinally towards the second fixed element 12, by telescopically sliding the tubular element 13 in the bore, to make the end of the tubular element 13 provided with the second movable element 14 project further out of the bore.

In this example, the second drive part 16b has been moved, e.g. rotated. The transmission 8′ transforms the motion of the second drive part 16b into a movement of the movable parts 3c,3d in the direction of expansion, in this example radially away from the axis of rotation of the drive part. In this example via a transformation of the rotating motion of the second drive part 16b into a linear motion of the second movable element 14. In an alternative implementation this transformation may be direct, but in the shown example intermediate movements are used, the rotating motion is transformed into a linear motion of the second drive part 16b itself, which results in a linear motion of tubular element 13 (which is blocked from rotating around the axis which extends in the direction of motion) and the linear motion of tubular element 13 moves the second movable element 14 rectilinearly. In some implementations, second movable element 14 can be a portion of the tubular element 13 or be mechanically coupled to the tubular element 13 and spaced apart from the second drive part 16b by the tubular element 13.

More specifically, the motion of the second drive part 16b results in this example in a motion of the second movable element 14 as follows. The second drive part 16b is in this implementation rotatable relative to the support structure, and as explained above, by rotating will move translationally, in the longitudinal direction of the implant body. The longitudinal movement of the second drive part 16b pushes the tubular element 13 to slide in the bore, which in this example is placed co-axially to the second drive part 16b, and is likewise tubular, which in turn pushes the second movable element 14 to the other element 12. The tubular element 13 is in this implementation translationally movable only, and blocked from rotating around its longitudinal axis relative to the support structure. As illustrated, in this example the tubular element 13 is pushed towards the second fixed element 12, out of the bore in the anchor body 4. For example, as the second drive part 16b is rotated (e.g. by the surgical tool), tubular element 13 may move longitudinally with respect to the support structure 2 and the elongate shaft 10. In this example, the second movable element 14 is immovably fixated relative to the tubular element 13 and therefore likewise movable translationally only.

FIG. 6 shows an expandable intra-osseous implant in a third expanded state. In the shown third expanded state, the implant is in a mixed expanded state which comprises both the first expanded state and the second expanded state. The implant 1 is fully expanded and all movable parts 6a-6d of all sets 3a-3d have been moved from their first position to the position most far away from the support structure to maximally project outwards from the implant, relative to the shape thereof in the non-expanded state. However, the third expanded state may refer to any state in which movable parts 3a-3d of both sets 3, 3′ of movable parts are expanded, either partially or maximally. The movable parts 3a-3d are illustrated in FIGS. 6A-C as being expanded to an equal extent and FIG. 6 illustrates each of movable parts 3a-3d in a maximally expanded state. It is, however, to be appreciated that movable parts 3a-3d need not be maximally expanded, or equally expanded. For example, the set 3 of movable parts 3a, 3b may be expanded to a greater or lesser extent than another set 3′ of movable parts 3c,3d. For example, neither of the subset of movable parts 3a, 3c of the sets 3, 3′ of movable parts may be maximally expanded.

In this example, as can be seen in FIG. 6A in the third expanded state, both sets 3,3′ are expanded in the same direction and the load bearing surfaces 6a-6d are in the longitudinal direction of the implant parallel.

FIG. 6C shows a cross-sectional view of the device 1 in the third expanded state. As shown, both the first transmission 8 and the second transmission 8′ have been actuated as explained with reference to FIG. 4 for the first state and with reference to FIG. 5 for the second state. However, in some implementations, the first transmission 8 and the second transmission 8′ may be actuated independently of each other, enabling the expansion of the first set 3 of movable parts 3a, 3b of a set 3 and second set 3 of movable parts 3c, 3d to be achieved to different extents.

An implant may be expanded with a surgical tool for engaging with a transmission of an expandable intra-osseous implant. FIGS. 7-10 show surgical tools 25,29 for engaging with a transmission of an expandable intra-osseous implant, such as described above. Referring to FIGS. 7C-E and 9C-E, there may be at least two surgical tools. The tools may for instance comprise a first surgical driver tool 25 for engaging with the first transmission 8 to drive movement of a first movable part of a set of movable parts. In the example of FIG. 7 such a surgical tool is present, and is shown as a stand-alone in FIG. 7C. The surgical tools may comprise a second surgical driver tool 29 may engaging with the second transmission 8′ to drive movement of one or more second movable parts. In the example of FIG. 9 such a surgical tool is present, and is shown as a stand-alone in FIG. 9C.

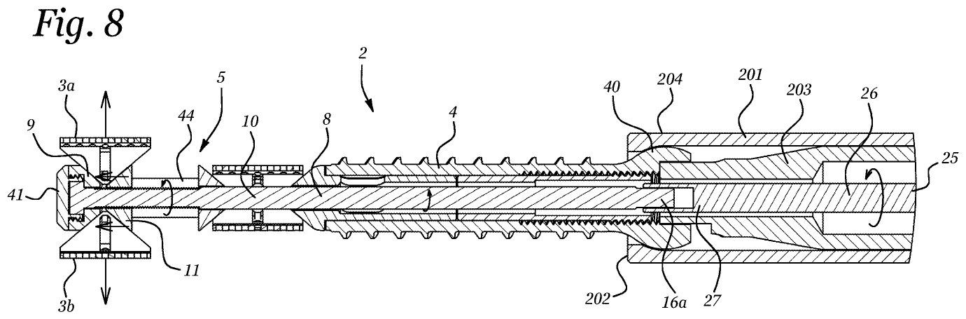

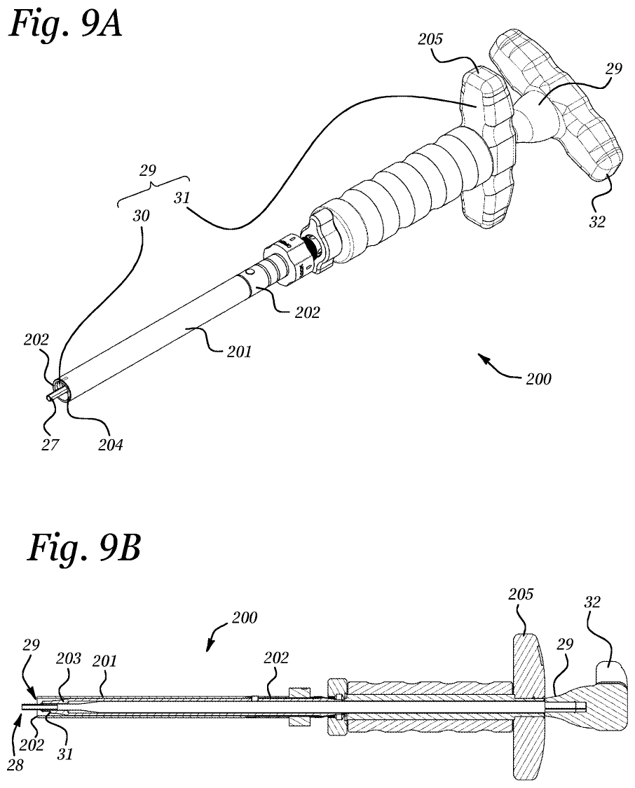

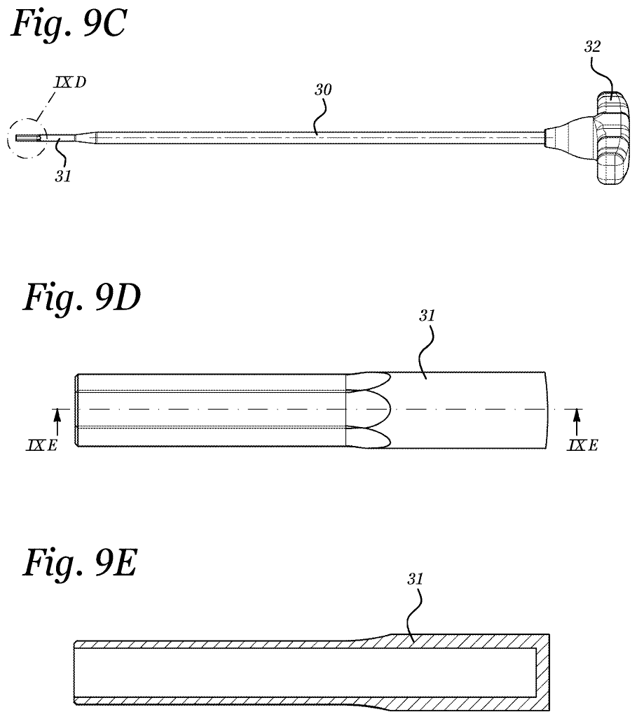

Referring to FIG. 7C-E, the first surgical driver tool 25 may engage with a tool interface 16 of device 1, in this example with drive part 16a. The first surgical tool 25 may comprise a first elongate part 26. In some implementations, the first elongate part 26 may engage with the first transmission 8 of the device 1, without engaging the second transmission 8′. First elongate part 26 may extent to a first engaging end 27. The first engaging end 27 may directly or indirectly couple or mate with elongate shaft 10 of the device 1, in this example by mating the end 27 with first drive part 16a, as illustrated with the arrows in FIG. 8. Rotation of the first engaging end 27, when coupled with elongate shaft 10, may cause rotation of elongate shaft 10. In some implementations, the first engaging end 27 may have a recess with a shape conforming to the head of the elongate shaft 10, and may be placed over the head. For example, first engaging end 27 may comprise a hex-shaped recess, which may engage with a hex-shaped head (e.g. hex-shaped drive) of elongate shaft 10 or tool interface 16, as illustrated in FIG. 7D and E. A first handle 28 is mechanically or electrically coupled to first engaging end 27, such that rotation of the first handle 28 (e.g. by the hand of an operator of the tool) causes a rotation of the first engaging end 27. In this example, the elongate part 26 connects the first handle 28 to the first engaging end 27 to transfer torque from the handle 28 to the first engaging end 27.

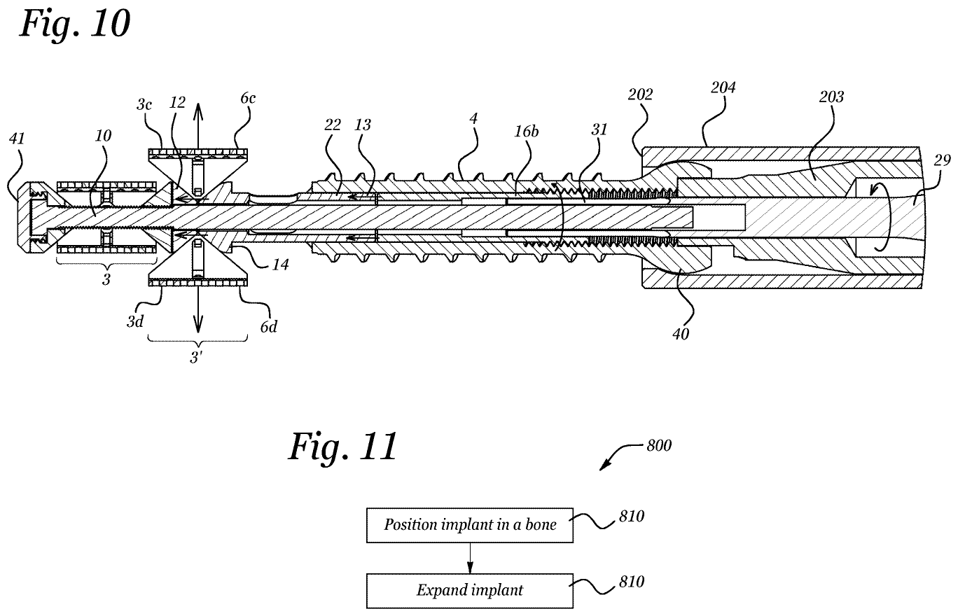

Referring to FIG. 9C-E, the second surgical driver tool 29 may engage with a tool interface 16 of device 1, in this example with second drive part 16b. The second surgical tool 29 may comprise a second elongate part 30. In some implementations, second elongate part 30 may engage with second transmission 8′ of the device 1, without engaging the first transmission 8. Second elongate part 30 may extent to a second engaging end 31. A second handle 32 is mechanically or electrically coupled to second engaging end 31, such that rotation of the second handle 32 (e.g. by the hand of an operator of the tool) causes a rotation of the second engaging end 30. The second engaging end 31 may directly or indirectly couple or mate with tubular body 13 of the device 1, via second drive part 16b. As illustrated with the arrows in FIG. 10, rotation of the second engaging end 31, when coupled with tubular element 13, may cause a translational movement of tubular element 13, to telescopically slide in the bore and move the end of the tubular element 13 on which the second movable element 14 is provided towards or away from the second fixated element. In this example, rotation of the second engaging end 31 causes a rotation of the second drive part 16b which, due to the threaded coupling with the bore, is translated in a translational movement of the second drive part 16b in the longitudinal direction. The second drive part 16b lays in the bore abutting to, but not engaging on, an end of the tubular element 13 and thus pushes the tubular element 13 to telescopically slide in the bore without transferring the rotational movement on the tubular element 13. In some implementations, the second engaging end 31 may be configured to fit into a recess in a head of the elongate shaft 1 tubular element 13, and may be shaped to correspond with a shape of said recess. For example, as illustrated in FIG. 9D, second engaging end 31 may comprise a hex-shaped or other not circle-cylindrical portion, which may engage with a hex-shaped or other not circle-cylindrical recess (e.g. hex-shaped drive) of tubular element 13 or drive part 16b. As illustrated in FIG. 9 E, the second engaging end 31 may have a blind-hole in which the first drive part 16a fits without engaging with the drive part 16a, while the outside of the second engaging end 31 engages with the recess in the tubular element 13. In this example, the blind-hole has a cylindrical shape with a diameter which is larger than the drive part 16a. The second engaging end 31 can therefore rotate the drive part 16b and drive the second transmission 8′ without rotating the drive part 16a (and without driving the first transmission 8).

In these example, the surgical tools 25,29 are shown as part of a surgical tool set 200 for orthopaedic surgery but in an alternative they may be provided separately. FIG. 7A-7B illustrate the tool set 200 with a first surgical driver tool 25 which can engage wit the first transmission 8. FIG. 9A-B illustrate the tool set 200 with a second surgical driver tool 29 which can engage wit the second transmission 8′.

Referring specifically to FIGS. 7A,7B and 9A,9B, in addition to the surgical tools 25,29, each of the surgical tool sets 200 comprises a hollow tubular body 201 with open ends 202, for providing the surgical driver tool 25 access to the outside of the bone and to the tool interface 16. In an implementation, the tubular body 201 is a tubular retractor. The open ends 202 may be located at opposite longitudinal ends of the hollow tubular body 201. At one of the open ends 202 the surgical tool may engage with the drive tool interface 16 of device 1, as is illustrated in FIGS. 8 and 10 respectively. The elongated part 26,30 may fit into the hollow tubular body 201. The surgical driver tool 25,29 may positioned to extend through the hollow tubular body 201. The engaging end 27,31 is when the surgical tool is in an engaged state, located at a distal end of the hollow tubular body 201 and engages with the transmission 8,8′ of the implant 1.

The tool set 200 may comprise a sleeve 204 with an internal shape conforming to an outer shape of the anchor body 4 at a projecting end of the anchor body 4. In this example the projecting end is the proximal end 40 of the implant. When the implant 1 is placed in the bone, the projecting end projects out of the vertebra 100. Both the internal shape and the outer shape have an un-round cross-section perpendicular to a longitudinal direction of the anchor body. Thus, by positioning the sleeve 204 over the projecting end, a force can be exerted on the body of the implant 1, which in these example is formed by the anchor body 4. In the shown examples, the sleeve 204 is formed by a longitudinal end of the hollow tubular body 201. As can be seen in FIG. 2A, the proximal end may for example have flattened side planes and have a cross-sectional shape (taken perpendicular to the longitudinal direction of the anchor body 40) with non-circular segments. The internal shape of the sleeve 204 may have a similar shape.

As illustrated, at the other open end 202 of the hollow tubular body 201 other parts of the surgical tool set 200 may be provided and/or inserted into the hollow tubular body 201 and/or mounted onto the hollow tubular body 201. In this example, a retainer 203 is for example inserted in the hollow tubular body. As explained in more detail with reference to FIGS. 8 and 10, the retainer 203 has a retaining state in which the retainer 203 engages on the implant 1 to hold the implant in position when the surgical driver tool 25,29 engages on the transmission 8,8′ and drives the movement of the movable part. The retainer 203 is fixated onto the hollow tubular body 201 such that it is unmovable relative to the tubular body. When the engaged retainer 203 is held unmovably in position relative to the bone, e.g. against rotational forces, both the implant 1 as well as the hollow tubular body 201 are held unmovably in position relative to the vertebra 100. In this example, the retainer 203 is mounted on the hollow tubular body 201 and fixated by a bayonet-mount at the other open end 202 but other fixations are likewise possible.

The retainer 203 may for example engage on the head or proximal end 40 of the anchor body 4. As illustrated in FIG. 2A, the head may a shape which can mate with the end of the retainer 203. In this example, the head is shaped as a torx-screw head and the retainer 203 has a torx-screw, but other shapes, such as recesses with a non-circular cross-section in which the end of the retainer 203 can be positioned may alternatively be used. As illustrated in FIGS. 8 and 10, the retainer 203 may be placed in the hollow tubular body 201 with the retainer 203 end mating with the head or proximal end 40, while a surgical driver tool 25,29 engages on the transmission. This allows to maintain the head in position while expansion of the implant is driven by the surgical driver tool 25,29.

The retainer 203 has a head on a grip 206 can be mounted. As shown, the grip 206 comprises a sleeve with a retainer handle 205 which can be taken into the hand of an operator of the device to exert a counterforce that maintains the retainer 203, and hence the implant 1, in position against the forces acting on the implant while the surgical driver tool 25,29 is driving expansion of the implant 1. As shown, the grip is mounted on the retainer had by a clamp 207 can be provided, in this example the head has an threaded part on which the clamp can be screwed.

As can be seen in e.g. FIGS. 7B and 9B, the retainer 203 may have a hollow tubular inside which extends, when the retainer 203 is positioned in the hollow tubular body 201, parallel to the hollow tubular body 201, inside the hollow tubular body 201. The hollow tubular inside has at each longitudinal end an opening The surgical driver tool 25,29 fits with the engaging end 27,31 positioned at the distal end in the hollow tubular inside. In an implementation, when the surgical driver tool 25,29 is engaged with the transmission, the tool 25,29 is movable relative to the retainer 203, to drive the expansion of the implant 1, with the engaging end 27,31 engaged with the transmission 8,8′. In the implementation shown, as indicated with the arrows in FIGS. 7B and 9B the surgical driver tool 25,29 is rotatable relative to the hollow tubular body 201 (and the retainer 203) to drive the expansion, but in other implementations this may e.g. be a translational movement, depending on the type of transmission and drive part on which the engaging end engages.

The elongate part 26,30 of a surgical driver tool 25,29 may be coaxially arranged with the hollow inside of the retainer 203 and/or with the hollow tubular element 201. That is, first elongate part 26 may share a central longitudinal axis with the retainer 203 and/or with the hollow tubular element 201. In particular, elongate part 26,30 may be arranged to rotate independently around the shared axis relative to the retainer 203 and/or the hollow tubular element 201. That is, rotation of the elongate part 26,30 (e.g. manually by rotating the handle 28,32) need not cause rotation of the retainer 203 and/or cause rotation of the hollow tubular element 201. Similarly, rotation of the retainer 203 and/or with the hollow tubular element 201 (e.g. by manual manipulation of the handle 205) need not cause rotation of the elongate part 26,30.

The surgical tool or tool set may be provided in a kit for orthopaedic surgery. Such a kit may comprise an implant, such as described above, and a surgical tool or a surgical tool set.

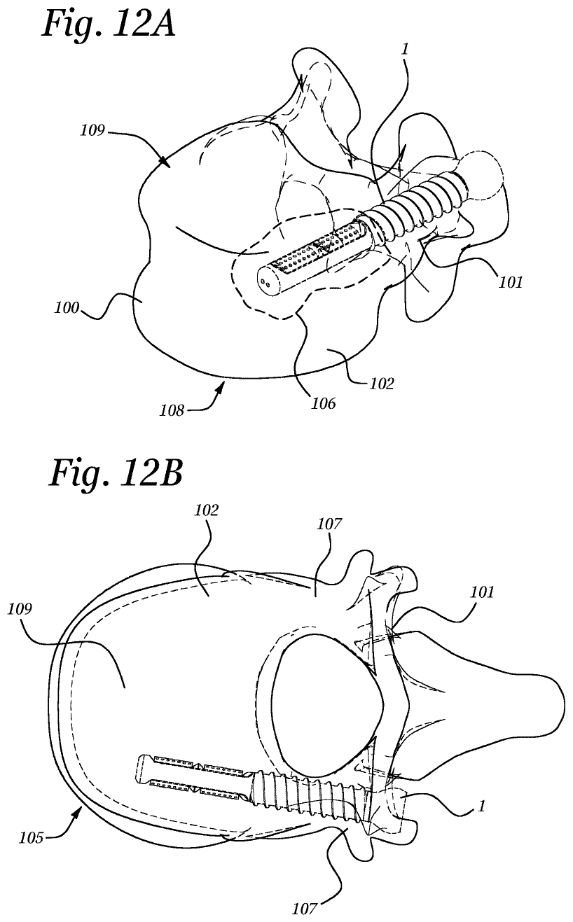

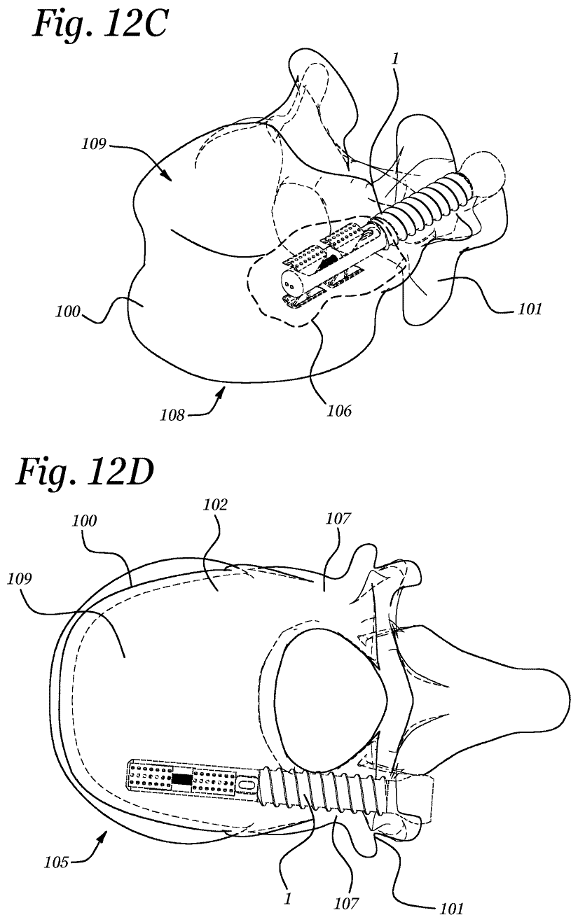

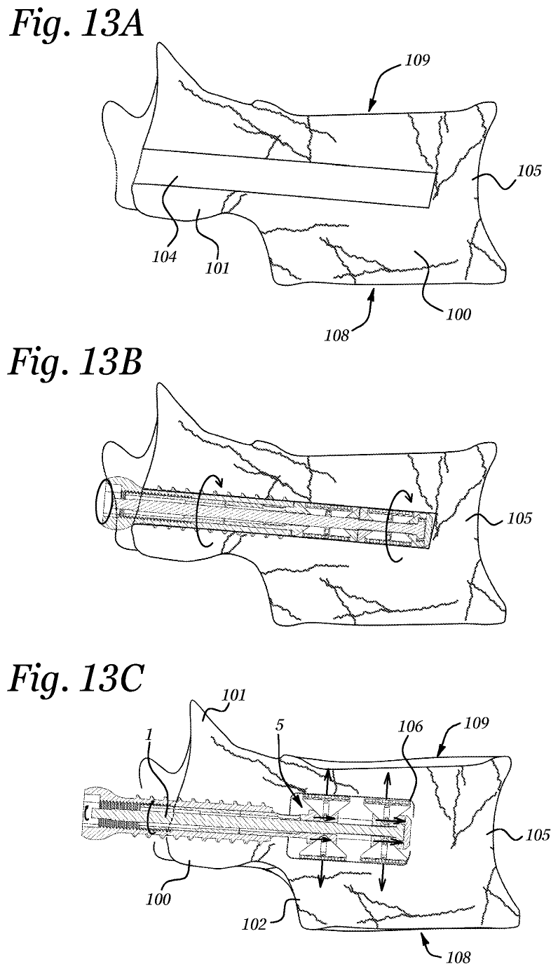

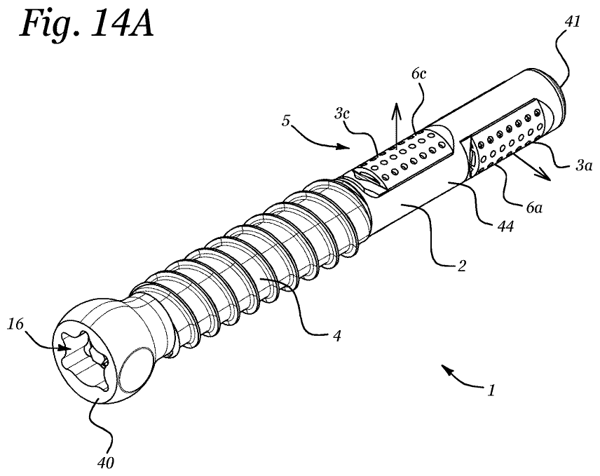

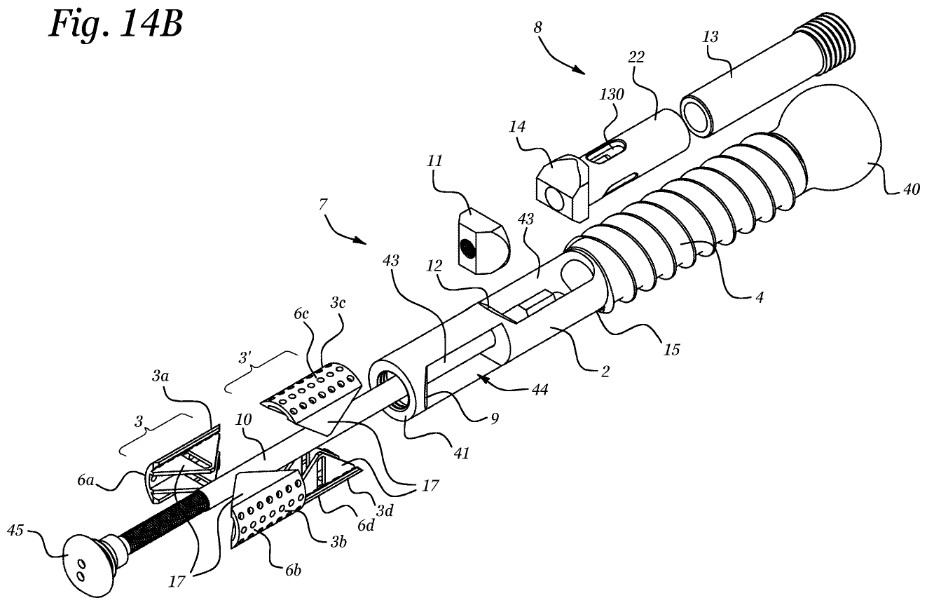

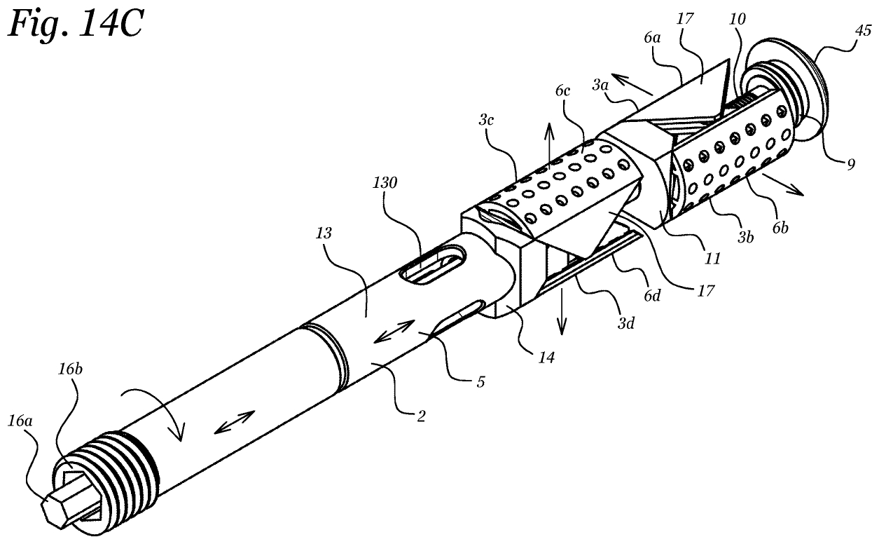

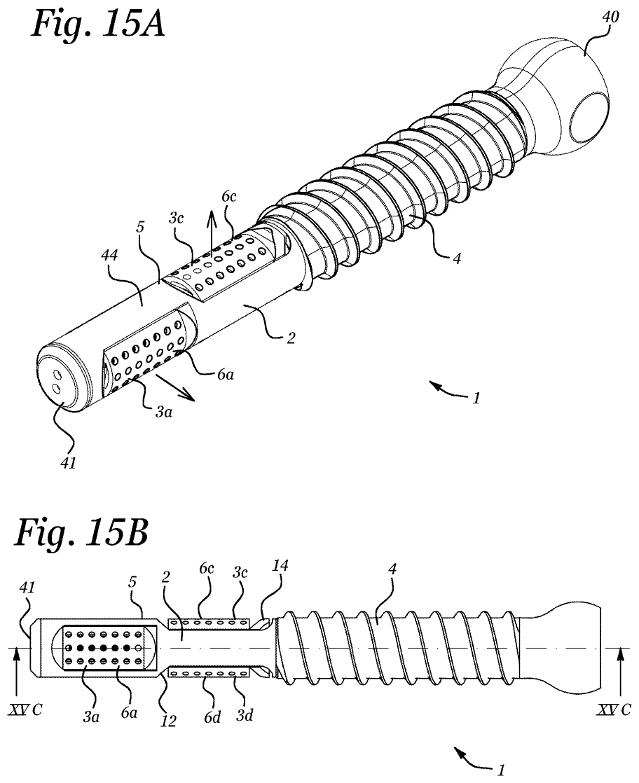

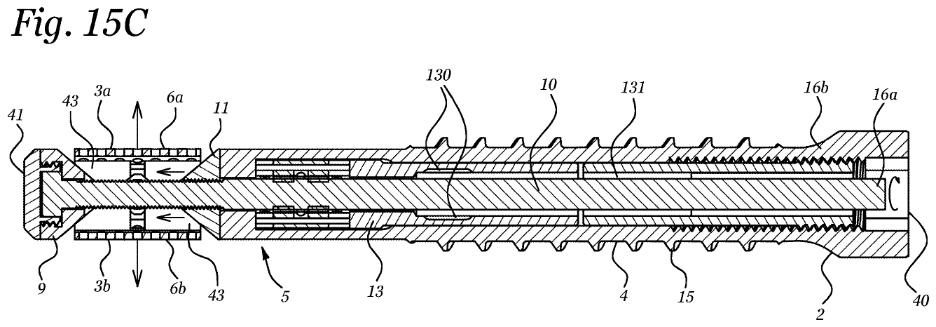

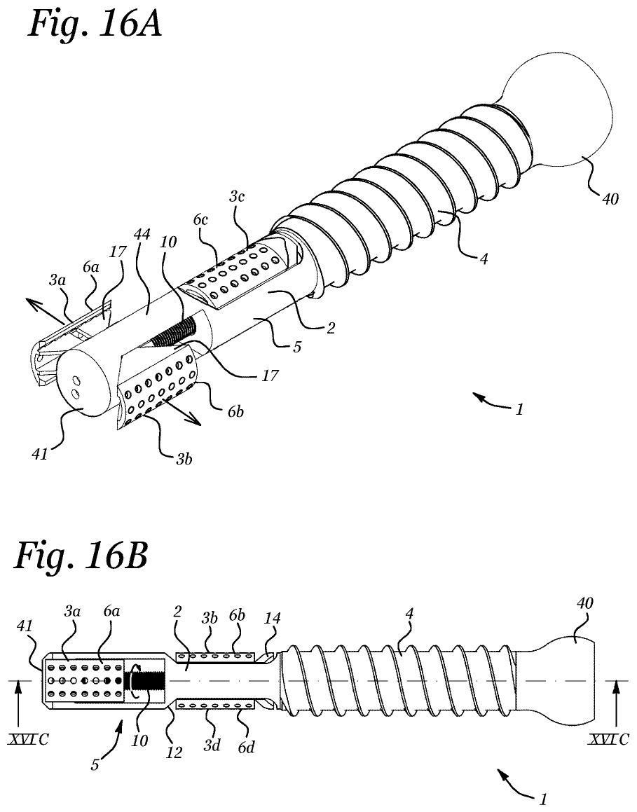

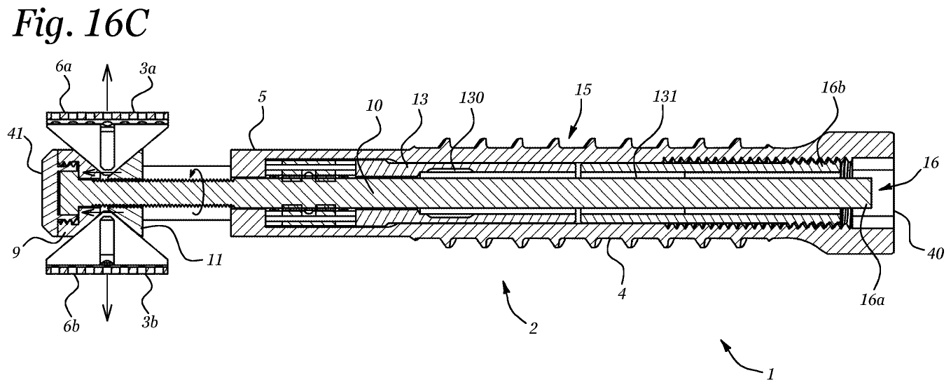

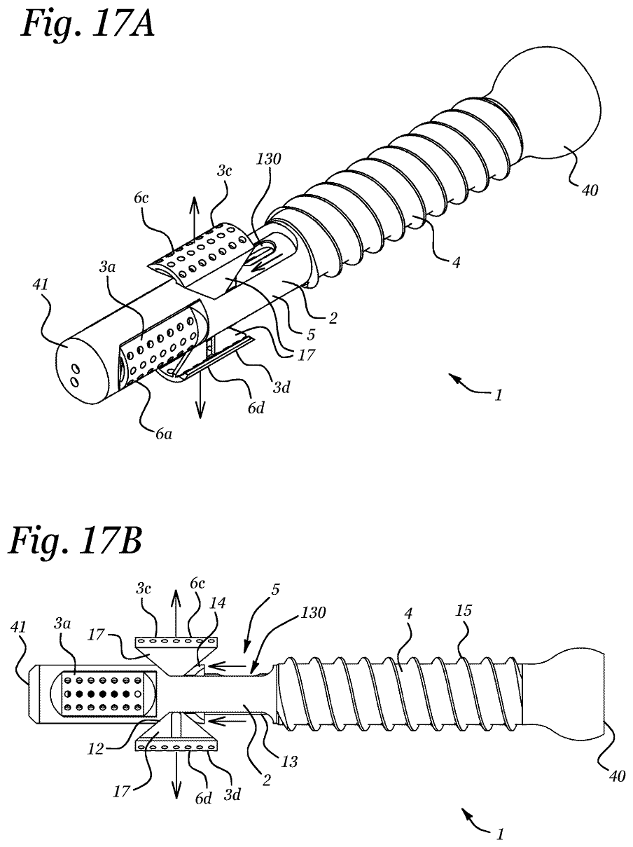

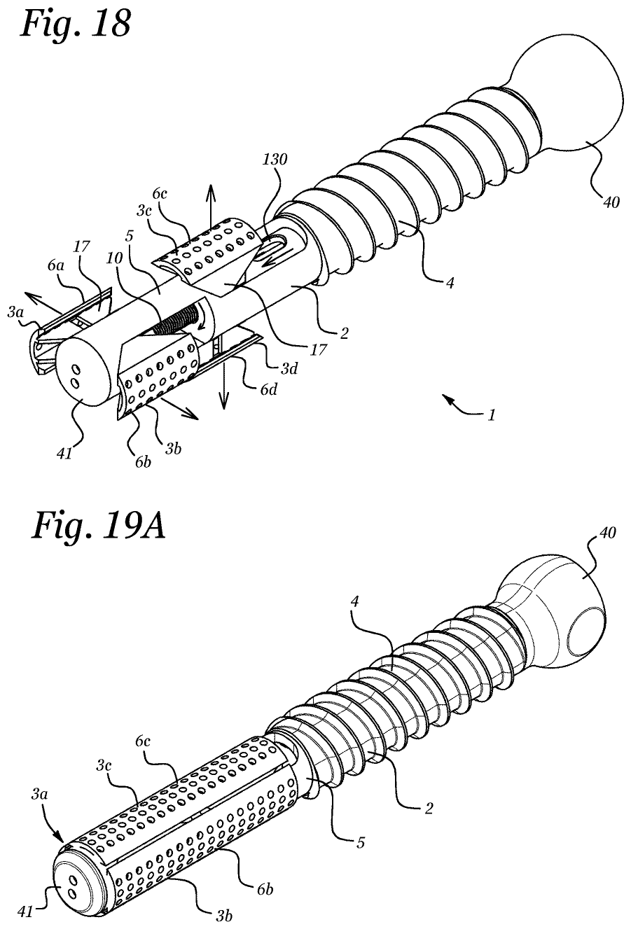

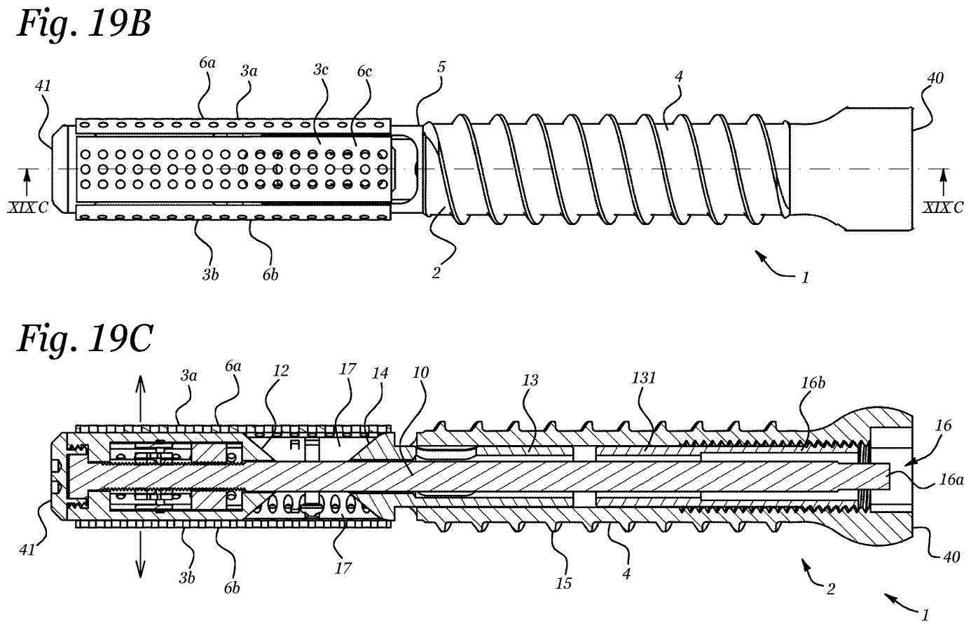

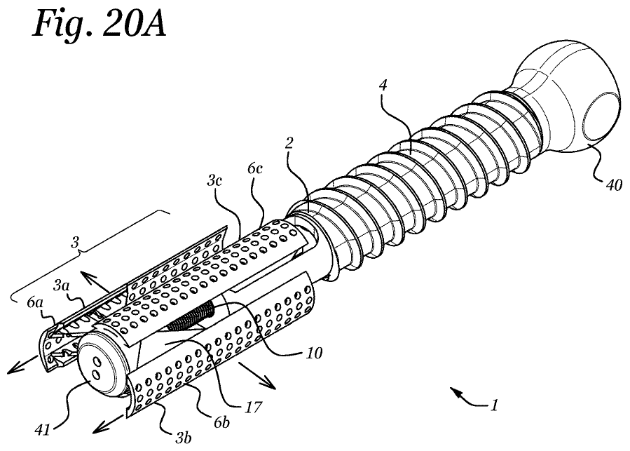

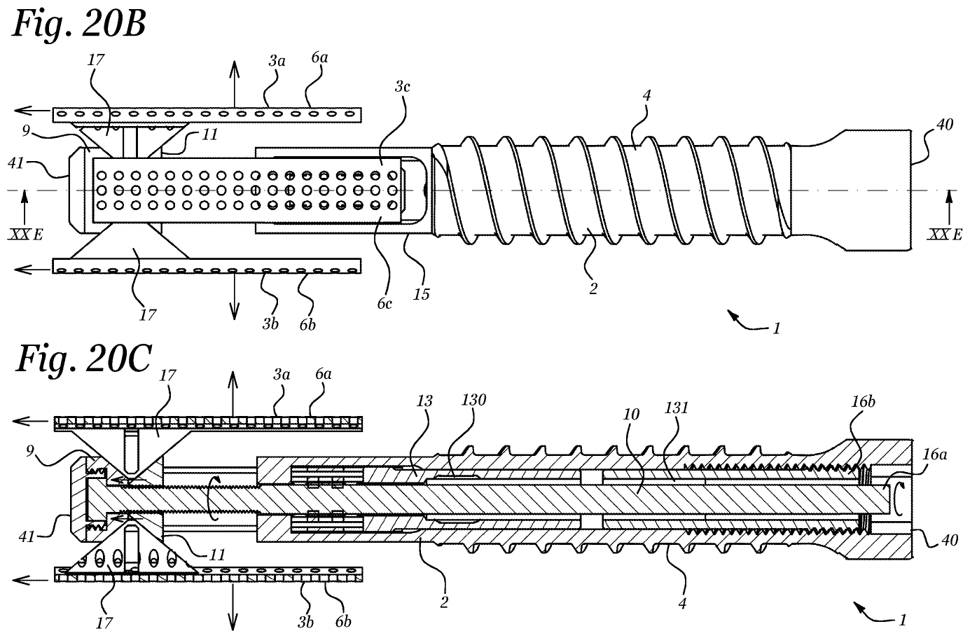

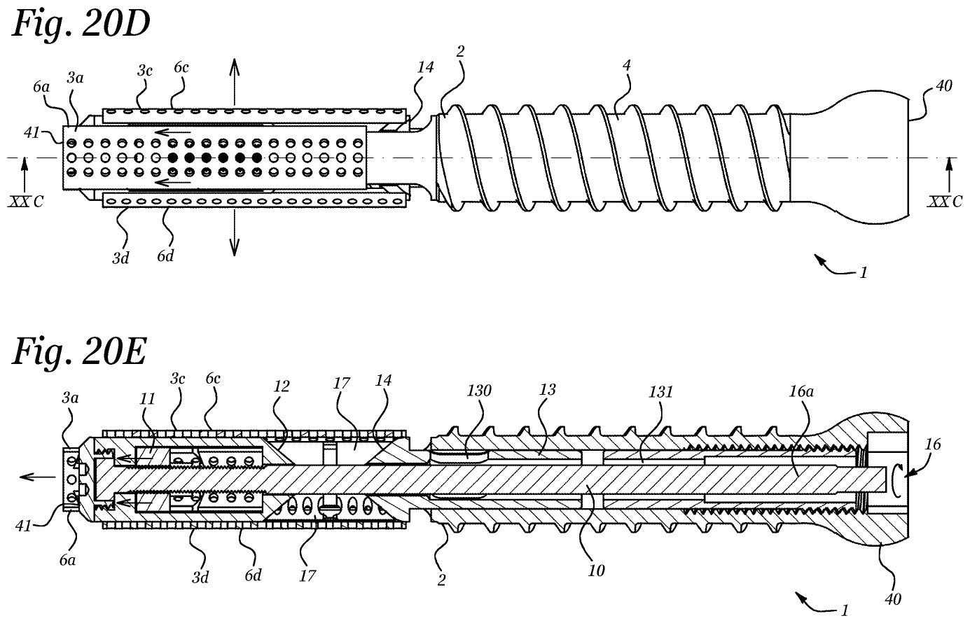

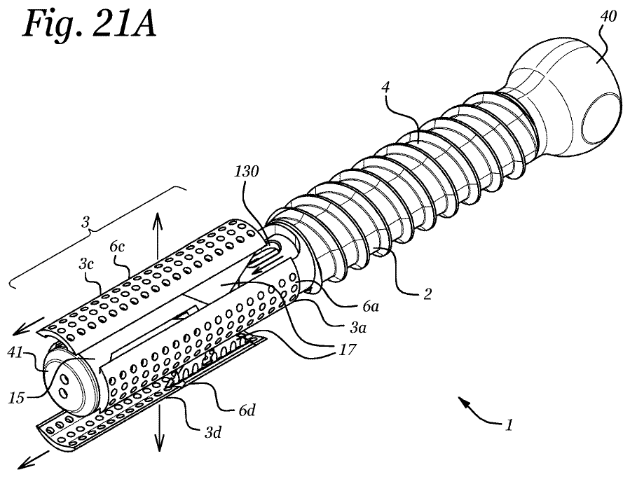

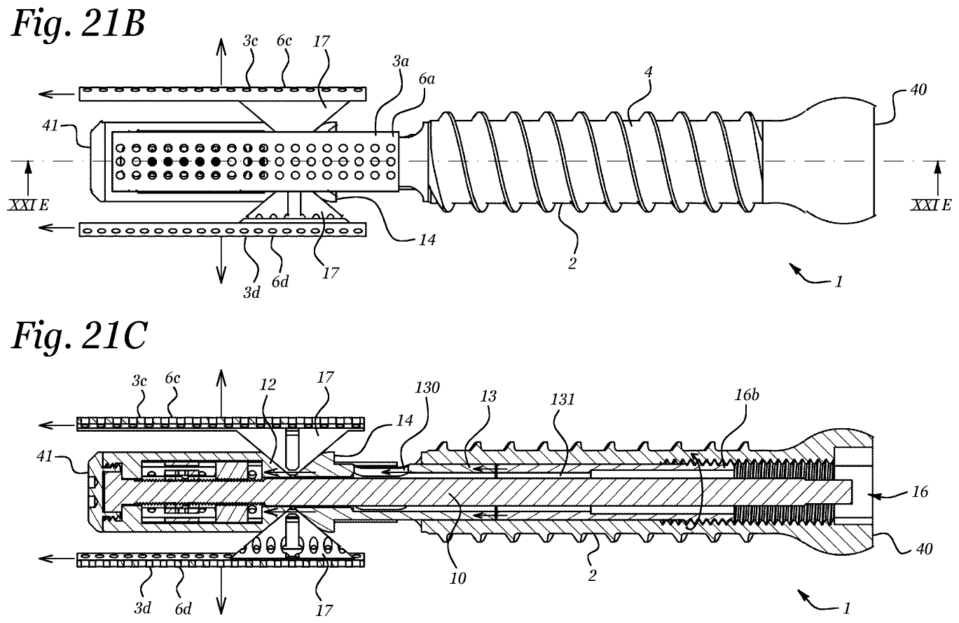

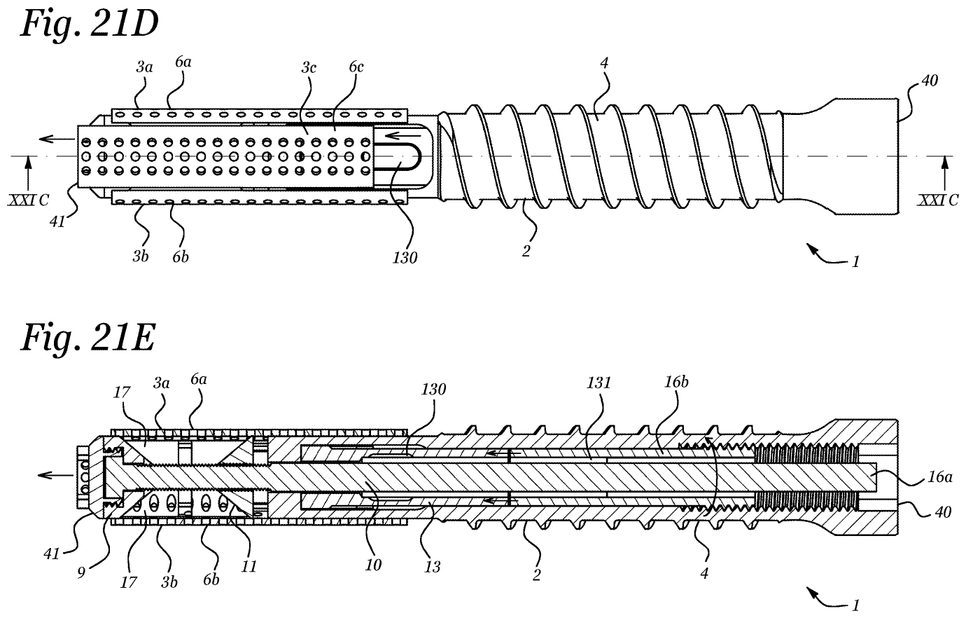

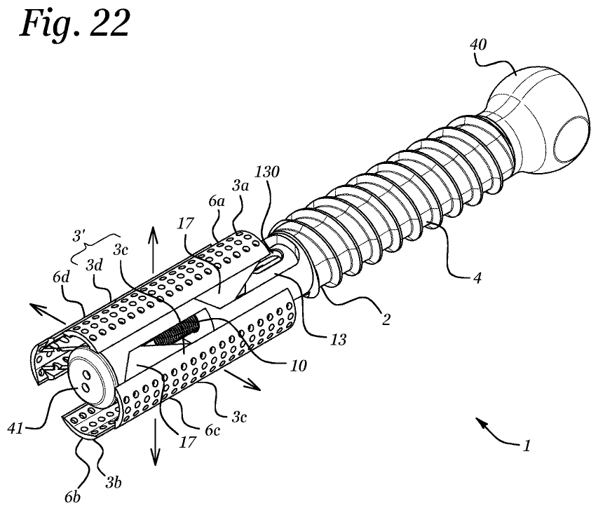

Referring to FIGS. 11-13, the implant may be used in a method of orthopaedic surgery of a living, human or non-human, mammalian body. FIG. 11 shows a flowchart of an example of a method 800 of orthopaedic surgery. The method may be performed by a surgeon or other qualified healthcare practitioner operating an implant 1, such as the example of FIG. 2 or of FIG. 14 or of FIG. 19. The orthopaedic surgery may be to treat a condition of the vertebra, such as a fracture or weakness, for instance a compression fracture, wedge fracture, burst fracture and/or flexion-distraction fracture, e.g. as described above with reference to FIG. 2 for example. The vertebra may be fractured in an anterior part, or region, and/or a posterior part, and/or a central part of the vertebral body for instance.

The method 800 may comprise, as illustrated in the flow chart with the block 810 “POSITIONING IMPLANT IN BONE”, positioning an implant (e.g. device 1) in a bone, such as in a vertebra, for example a human vertebra. In some implementations, positioning the device 1 may be preceded by preparing the bone for placement of the implant. For example, an intraosseous cavity may be made inside the bone. For example, a tubular retractor may be placed after the incision has been made, to provide surgical access to the bone and prepare the bone. During the positioning operation, the device 1 may be in a non-expanded state, or in a substantially non-expanded state.

To position the implant, for instance an incision may be made in the mammalian body or other access to the bone be created and the implant 1 be inserted in the bone. Positing the device 1 may comprise orienting the implant 1 in addition to placing the implant 1 at a desired location in the bone. For instance, a healthcare practitioner operating the implant 1 may have determined the part of the bone to be supported by the implant and selected a set of movable parts best located to support that part. When positioning, the implant may be oriented such that the respective load bearing surfaces of the selected set 3,3′ face towards the load acting on that part. E.g. in the third example, the surgeon or other healthcare practitioner may e.g. deem a peripheral part of the vertebral end plate requiring support, such as in case of a vertebral wedge compression, and orient the first set 3 to face the vertebral endplates with the second set 3′ facing side-wards. Vice versa, the surgeon or other healthcare practitioner may deem a more centrally located part of the vertebral end plate requiring support, such as in case of a collapse of the superior vertebral end plate, and orient the second set 3′ to face the vertebral endplates with the first set 3 facing side-wards.

The method 800 may further comprise expanding the implant, as indicated with block 820 “EXPANDING IMPLANT. Expanding the device 1 may comprise actuating a movement of one or more movable parts 3a-3d of the sets 3 of movable parts as described above. Generally speaking, expanding the implant 1 may comprise transferring the implant from the non-expanded state, such as illustrated in FIG. 3, to a desired final state. The final state can e.g. be the first expanded state, such as illustrated in FIG. 4, the second expanded state, such as illustrated in FIG. 5, or a mixture of those, such as illustrated in FIG. 6.

In some implementations, expanding the device 1 may comprise expanding each movable part 3a, 3b of at least one set 3 of movable parts to an extent determined by the surgeon. A first movable part 3a of the set 3 of movable parts may be expanded to a lesser or greater extent than a second movable part 3b of the set 3 of movable parts, or the movable parts 3a, 3b of the set 3 of movable parts may be expanded to an equal or substantially equal extent. The device 1 may be expanded within the cavity 106 such that the load resisting surface of the movable parts 3a, 3b of at least one set 3 of movable parts abut, e.g. push, against the wall(s) of the cavity within the vertebra, in the direction of expansion.

In some implementations, expanding the device 1 may comprise first actuating a movement of a first movable part 3a of a first set 3 of movable parts, thereby expanding the first movable part 3a, and subsequently actuating a movement of a first movable part 3c of a second set 3′ of movable parts, thereby expanding the movable part 3c, or vice versa. For example, expanding the device 1 may comprise expanding each movable part 3a and 3c in turn, for example in a step-wise approach. That is, first movable part 3a may be first partially expanded (e.g. to partially support the load), the second movable part 3c may subsequently be partially or fully expanded (e.g. expanded to a desired final position), and the first movable part 3a may then be expanded to a desired final position. Such a step-wise method of expansion may improve how the load is distributed and may reduce stress on the bone (e.g. vertebra) during the orthopaedic surgery. Alternatively, for instance when using an implant as in the third or fourth example, for instance a first set may be expanded to the final position to fixate the implant in position, e.g. by a side-wards expansion and subsequently expanding a second set to a final position to support the bone.

Referring to FIGS. 12-13, as an example of a bone, the implant 1 is shown anchored in a human vertebra 100. The vertebra 100 comprises a vertebral arch 101 and a vertebral body 102. The vertebral arch 101 comprises pedicles 107 and the vertebral body 102 has endplates 108, 109. Further indicated in those FIGs. is an intra-osseous cavity 106 in the vertebral body 102. The cavity is located close to the surface of the bone on which the external load acts, in this example close to the vertebral endplate 108,109. For example, 6a-6d mm or less, such as 4 mm or less, such as 3 mm or less of bone tissue may be present between the top or load resisting surface 6a-6d of the implant and the endplate 108,109. This allows an elastic or plastic deformation of this tissue by the load resisting surface 6a-6d pushing against the vertebral endplate 108,109 upon expansion and accordingly allows to reduce the risk of bone fracture or collapse when expanding the implant 1 (e.g. to partially or completely restore the vertebral height). For instance, 1 mm or more, such as 2 mm or more, for example 3 mm of tissue may be present between the top surface 30 and the vertebral endplate. This reduces the risk that the implant 1 pierces through the tissue and becomes exposed during expansion or post-surgery.

Additionally, as illustrated, the distal end 41 of the implant 1 can be positioned close to the anterior wall 105 of the vertebra 100. For instance, the implant 1 may be positioned such that there is 1 mm or more, such as 2 mm or more, such as 3 mm or more of space, e.g. with spongy bone material, left between the distal end 41 and the anterior wall. Preferably, this space is 8 mm or less, such as 6 mm or less, for example 6a-6d mm or less. This allows to avoid piercing of the anterior wall by the implant 1.

The implant 1 can be placed with the expandable part of the implant 1 in the cavity. The expandable structure can then be expanded by exerting a moving force on the drive part 16a, 16b. The position of the implant 1 may be determined prior to expansion, for instance, via imaging techniques well known in the art, so as to ensure the expandable part of the implant 1 is fully inside the vertebral body 102 and e.g. is not in the pedicle, to ensure that an anchoring part is in the pedicle or that the implant is in a desired transpedicular position or an extrapedicular position, for instance. In case the implant 1 is provided with a bone anchor, the distal end 41 may be inside the cavity while the proximal end 40 may be outside the cavity and the anchor body 4 is anchored in the part of the vertebra 100 outside the cavity prior, during or after the distal end 41 is positioned in the cavity.

As illustrated in FIG. 13A, for example a cannula 104 may extend through the vertebra via which the implant 1 is placed in the bone, as illustrated in FIG. 13B and C. In this example, the cavity 106 is located at the end of the cannula and has been expanded by expansion of the implant. Thus, in this example, an initial cavity was prepared through the cannula and subsequently expanded, but the cavity may be prepared in another manner. For instance, prior to placing the implant the dimension of the cavity may have been increased using suitable orthopeadic instruments or the cavity may be prepared via another access than the cannula through which the implant 1 is placed.

As illustrated in FIG. 13B, the implant can for example be anchored in the pedicle by rotating the entire implant 1 and thereby screwing the anchor body 4 in the cannula 104, until the expandable structure 5 is at the desired depth in the vertebral body 102, and the load resisting surfaces 6 oriented as the medical practitioner deems appropriate. The implant 1 is then in the position and in the state illustrated in FIG. 13B and can be expanded.

With the shown example, anchoring the implant 1 and expanding the expandable structure 5 may be performed as separate steps. This allows to increase the control over the torque and/or pressure exerted at the interface between the load resisting surface 6a-6d and the vertebra 100. For example, the implant 1 may be inserted in a pre-made cannula 104 in the vertebra 100 with the distal end 41 first, until the profiled part of the anchor body 4 enters the cannula 104. Up to this point in time, the implant 1 may e.g. be slid, without rotation. Upon further insertion, the anchor body 4 starts frictionally anchoring in the cannula 104. In this example, by rotating the implant 1 the anchor body 4 will tap a threaded wall in the cannula 104, and this thread-forming insertion can then be continued until the distal end 41 is at the desired depth in the cannula 104. During this, the implant 1 remains in the non-expanded state. When the implant 1 is at the desired depth and the load resisting surface(s) 6a-6d are oriented to face in the desired direction, a force is exerted on the respective drive part 16a, 16b, which as elucidated above is transferred to the expandable structure 5 to expand one or more of the sets of movable parts of the implant 1. The implant 1 is then in an expanded state, as illustrated in FIG. 12C, D and 13C. In this example, the implant is in an expanded state in which two sets are expanded and as shown, the expansion is obtained by a rotating movement of the first drive part 16a which is transferred to the first transmission 8, and a rotating movement of the second drive part 16b which is transferred to the second transmission 8′ of transmission and transformed in a translational movement of the elements 9,11,12,14. This translational movement is transformed by the elements 9,11,12,14 in the movement of the movable parts 3a-3d in the direction of expansion d as previously explained.

As can be seen in FIGS. 12 and 13, the implant 1 can occupy in the expanded state of the device at least partially the volume of the intra-osseous implant cavity 106. Since the cavity 106 is at least partially or completely filled up the need to e.g. inject bone cement is obviated. However, it will be apparent that bone cement may still be applied, for instance for other purposes than filling the cavity and repairing fractures, e.g. bone cement may be provided at the contact interface between the device 1 and the wall, to stabilize and fixate the device 1 to the wall of the cavity 106. As can be seen more clearly in FIGS. 2B and 2E for instance, the implant may comprise a tubular element 13 with one or more than one passages 130 in the lateral wall of the element 13, between the inside and the outside of the tubular element, via which bone cement or another fluid can be injected into the cavity. The passages 130 may be connected to a fluid supply, and between the opening and the fluid supply a fluid channel 131 may be present. In the shown example for instance, the part of the elongate shaft 10 which in the exposed state lies in the tubular element 13 has a smaller outer diameter than the inner diameter of the tubular element 13 and in the radial direction of the tubular element 13 is at a distance from the inner wall of the tubular element 13. Thus a fluid channel 131 is present from the second drive part 16b to the passages 130. By connecting the second drive part 16b to a fluid supply, the fluid can thus be supplied to the passages 130. As illustrated in FIG. 3C, in the non-expanded state these passages 130 are covered and closed off. More specifically, in the non-expanded state, the passages 130 are covered and lay in the bore, inside the anchor body 4. As can be seen in FIG. 5C, in the expanded state at least one of the passages 130 is laid exposed and open, to allow a fluid communication between the inside of the tubular element 13 and the cavity 106 via which bone cement is injected. In this example, the passage 130 is exposed and opened when the second transmission 8′ is moved to expand the set of movable parts coupled to this transmission 8′. In this example, by rotating the tubular element 13, the tubular element 13 slides outwards, out of the bore in the anchor body 4, towards the distal end 41. The part of the tubular element 13 in which the passages 130 are provided then comes to project out of the anchor body 4, exposing the passages 130. This allows fluid to be injected into the cavity 106.

FIG. 14 shows a third example of an expandable intra-osseous implant. Like the first example of FIG. 1, this example comprises a support structure 2, one (or more than one, such as two) sets 3 of movable parts 3a, 3b, and a transmission system 7 (visible in 14D for instance). The third example is similar to the second example of FIG. 2 and differs in the following.

As can be seen, here the two or more sets 3,3′ are not only laterally offset but also off-set in the circumferential direction and in the circumferential direction the movable parts of the first set 3 are distanced from the movable parts of the second set 3′. More specifically, in the circumferential direction the movable parts of one set are interdigitated between the movable parts of another set. Said differently, in one set 3 a movable part 6a is separated in the circumferential direction from a successive movable part 6b of that set by a movable part 6c of the other set 3′. In the circumferential direction, the load resisting surfaces are alternating between a load resisting surface 6a, 6b of a first set and a load resisting surface 6c,6d of a second set. In this third example, the load resisting surfaces 6a-6d are still offset in the longitudinal direction. As can be seen, in this example the movable parts of the second set 3′ lay in the longitudinal direction further away from the distal end 41 than the movable parts of the second set.

Instead of the first set 3 and the second set 3′ being at the same expansion sides and having directions of expansion expanding in a direction parallel to each other, in the third example, the support structure 2 comprises at least two expansion sides which are at a non-zero angle smaller than 180 degrees, to each other. Here the sides are perpendicular, or substantially perpendicular but e.g. an angle larger than 45 degrees is also possible. In the third example, a first direction of expansion of at least one first expansion side is orthogonal, or substantially orthogonal, to a second direction of expansion of at least one second expansion side. Thus, the implant is in addition to the (opposite) first expansion directions belonging to the first set 3, expandable in two second (opposite) expansion directions belonging to the second set 3′. The second expansion directions extend in opposite, but in this example parallel, directions away from, e.g. perpendicular to, the first expansion directions.

The third example thus has at least two pairs of expansion sides, each pair for a specific set, facing away from each other. The direction of expansion of the first expansion side in a set is opposite, or substantially opposite, to the direction of expansion of the second expansion side in a set. The two pairs of expansion sides are perpendicular to each other. An expansion side from one set of movable parts 3a,3b is perpendicular to both the expansion sides in the other set of movable parts 3c,3d. As can be seen in FIG. 17 for instance, the shown example implant 1 is expandable in four directions and has four expansion sides.

In the example shown in FIG. 14, when the expandable structure 5 of the device 1 is positioned in the cavity 106 and expanded, load resisting surfaces 6a-6d may come to contact with walls of the cavity 106, and in this example push against two pairs of opposite walls of the cavity 106, with the two pairs of opposite walls being perpendicular to each other. That is to say, the implant 1 pushes on a pair of walls forming the top and bottom of the cavity 106 as well as a pair of walls that form the sides of the cavity 106. In this example the outside of the bone is the vertebral end plate 109 which interfaces with the inter-vertebral disc 103, and on which the spinal load acts. The device 1 thus supports the vertebra to resist the spinal load and to prevent collapse of the vertebra 100 whilst also having the capability of resisting a crushing force perpendicular to that of the spinal load and providing increase stability and reduced risk of the implant 1 slipping or moving within the cavity 106 during or after expansion.

As can more clearly be seen in FIG. 14A and B, in this example the implant body comprises for each set of movable parts 3,3′ separate openings 43, via which the movable parts admitted in the implant body can pass to expand the implant. The openings for the first set of movable parts are both laterally and circumferentially off-set relative to the second set of movable parts. More specifically, for each movable part a separate opening is provided in the body.

FIG. 15 illustrates a non-expanded state of the third example of FIG. 14 in. As illustrated in FIG. 14C, the implant may comprise multiple, for example four, expansion sides at which the implant 1 is expandable in a respective direction of expansion. Each of the expansion sides may be provided with at least one movable part from the sets 3, 3′ of movable parts 3a-3d. The load resisting surfaces 6a, 6b, of the first set 3 of movable parts 3a, 3b and the load resisting surfaces 6c, 6d of the second set 3′ of movable parts 3c, 3d may face in perpendicular directions. For example, the movable parts 3a, 3b of the first set 3 may be movable in a direction of expansion away from the expansion sides, whilst the movable parts 3c, 3d of the second set 3′ may be movable in a second direction of expansion away from the expansion sides, the first direction of expansion extending perpendicular to the second direction of expansion.

FIG. 16 illustrates the third example in a first expanded state, in which the first set is expanded. FIG. 17 illustrates the third example in a second expanded state, in which the second set is expanded. As can be seen, the first set expands in a radial direction, relative to the longitudinal axis of the support structure, extending away, in this example perpendicular, from the direction in which the second set expands. Upon expansion, in the second state, the expansion is both circumferentially and laterally offset relative to the expansion in the first state.

FIG. 18 illustrates the third example in a third expanded state, in which both the first set and the second set are expanded. As can be seen in FIG. 18, in the third expanded state the expandable structure 5 has a cross-like shape, extending and expanded radially in four different radial directions that are all either parallel but opposite or perpendicular to the other radial directions. As can be seen in FIG. 18C in the third expanded state, both the first transmission 8 and the second transmission 8′ have been actuated as explained with reference to the second example. In some implementations, the first transmission 8 and the second transmission 8′ may be actuated independently of each other, enabling the expansion of the first set 3 of movable parts 3a, 3b of a set 3 and second set 3 of movable parts 3c, 3d to be achieved to different extents.

In the example shown in FIG. 14, for instance, the implant 1 can be dimensioned to be positioned in a vertebral body and oriented to be expandable in a vertical direction, e.g. by expanding the first set 3, towards the vertebral endplates to push the bone 100 material outwards and support the vertebral endplates against the load acting on the spine of the mammal, (e.g. to partially or completely restore the vertebral height) as well as in a horizontal direction, e.g. by expanding the second set 3′, towards the sides of the vertebra 100 to hold the implant 1 firmly in position within the cavity 106 during expansion preventing slippage and ensuring the implant 1 expands as desired by the operator when initially positioning the implant 1 within the cavity 106. The expansion in the horizontal direction can also support the vertebra 100 against collapse from a crushing force perpendicular to that of the spinal load. Said differently, when correctly positioned in a human vertebra, the implant 1 can expand in the cranial-caudal direction, in the caudal-cranial direction, and in the lateral directions. For some implementations, 6 mm or less, such as 4 mm or less, such as 3 mm or less of bone tissue may be present between the side facing load resisting surface 6c, 6d of the implant and the side of the vertebra 100. This allows an elastic or plastic deformation of this tissue by the load resisting surface 6a-6d pushing against the vertebral side upon expansion and accordingly allows to reduce the risk of bone fracture or collapse when expanding the implant 1 (e.g. to partially or completely restore the vertebral width and securely hold the implant 1 in position within the cavity 106 wiring expansion avoiding slippage potentially caused by an uneven cavity top for example).

The implant may be provided in an assembled state. However, a kit of parts for assembling an implant 1 may be provided, comprising e.g. the components shown in FIG. 14B in an unassembled state or e.g. a kit comprising the assembled interior components of the implant 1 shown in FIG. 14D separate from the support structure 2 and anchor 4, for instance.

The fourth example shown in FIGS. 19-22 is another example of an expandable intra-osseous implant. Like the first example of FIG. 1, this example comprises a support structure 2, one (or more than one, such as two) sets 3 of movable parts 3a, 3b, and a transmission system 7 (visible in 14D for instance). The fourth example is similar to the third example illustrated in FIGS. 14-18 and described above and differs from the third example in the following.

Like the third example, the sets 3,3′ are off-set in the longitudinal direction. Each set 3,3′ of movable parts is located at a different distance, seen in the proximal-distal direction, from the distal end 41. More specifically, in a first set 3 the base 17 is located further away from the proximal end 40 and closer to the distal end 41 than in a second set 3′. That is to say that the bases 17 of the movable parts of each set 3,3′ of movable parts are longitudinally offset relative to the other set. In this fourth example however, the load resisting surfaces 6a-6d of the respective movable parts of 3a-3d project in the longitudinal direction from the base 17. In this example, the load resisting surface 6a-6d has a length which is about 2 times the width of the base (i.e. the dimension parallel to the lateral walls 20,21), but in alternative implementations the length may e.g. be 1.5 times the width or more or more than 1.2 times the width. In the shown example, the base is located at the side of the load resisting surface 6a-6d facing the longitudinal axis of the implant body. In this example, the base is located at either a proximal or distal end of the load resisting surface 6a-6d. Said differently, the load resisting surface projects in the proximal to distal direction or in the distal to proximal direction. The load resisting surface 6a-6d projects at one of the longitudinal ends of the base in the fourth example. Alternatively the load resisting surface 6a-6d can project from the base 17 at both of the longitudinal ends of the base 17 for instance, that is in the proximal to distal direction and in the distal to proximal direction.

In this example, the load resisting surfaces 6a-6d of a set 3 project towards the bases 17 of the movable parts of the other set 3′. The load resisting surfaces 6a-6d of a set may partially or completely overlap in the longitudinal direction with the load resisting surfaces of the other set, and e.g. partially or completely extend in the circumferential spaces between the load resisting surfaces of the other set. Said differently, the bases 17 in a set may be off-set in the longitudinal direction relative to the bases 17 in the other set, whereas the load resisting surfaces 6a-6d of the sets are interdigitated, e.g. in circumferential direction around the longitudinal axis of the implant body alternating between a load resisting surface of a first set and a load resisting surface of a second set.

The load resisting surfaces 6a-6d can e.g. be of a length in the proximal-distal direction that completely covers the longitudinally offset of the bases of the sets 3, 3′ of movable parts. Therefore, in the shown example, there is in the non-expanded state no observable distance (off-set), seen in the proximal-distal direction, between the load bearing surfaces 6a-6d. Said differently, the load resisting surfaces 6a-6d of both sets are located in the longitudinal direction at the same location. In some implementations, shown in FIGS. 20 and 21 for example, in some or more of the states of the implant, in these examples in the first and second expanded states, the load resisting surfaces 6a-6d may not be located in the longitudinal direction at the same location. In this example, upon expansion of a set, the position of the load resisting surfaces 6a-6d of that set is shifted because they are moved in the longitudinal direction (in this example in addition to the radially outwards movement). In this example, there is still an overlap in the proximal-distal direction, between the load resisting surfaces 6a-6d of the sets 3, 3′ of movable parts as well as a difference in distance, seen in the proximal-distal direction, from the proximal end 41 of the load resisting surfaces 6a-6d of the sets 3, 3′ of movable parts.

In some examples, the projecting part of the load resisting surface 6a-6d may be bendable and bend, such as elastically deform (that is flex) under the load. In such a case, the base 17 of the respective movable part may serve as a fulcrum. Instead of flexing, bending with a plastic deformation is likewise possible. In such a case, the project parts of one set may extend in a direction opposite to the projecting part of another set, which allows to further broaden the range of

In the fourth example, the load resisting surface 6a-6d lies radially at a distance from the axis of the support structure 2 and the base 17 extends from the load resisting surface 6a-6d towards that axis. The base 17 is admitted in the implant body in the non-expanded state, and the movable part can expand by passing the base 17 through the opening 43. As can e.g. be seen in FIG. 19C, in the non-expanded state a load resisting surface 6a-6d can project in the longitudinal direction beyond the opening and cover a part of the outside of the implant body, i.e. overlap with a part of the implant body (in this example of the tubular body forming the support structure 2). The load resisting surface 6a-6d may in the non-expanded state contact the outer surface of the implant body in the region of overlap, as in FIG. 19C. Thereby, the projecting part of the load resisting surface 6a-6d is supported in the non-expanded state. This allows to reduce undesired deformation thereof during positioning of the implant, However, in another implementation there may in the radial direction be a, smaller or larger, gap between load resisting surface 6a-6d and the body of the support structure 2. For example the radial distance from the axis of the load resisting surface 6a-6d may be larger than the diameter of the support structure.

In this example, the load resisting surfaces 6a-6d are oriented parallel to the longitudinal direction of the implant 1, at a distance from the longitudinal axis. The load resisting surfaces 6a-6d are at an angle relative to each other. Said differently, in the circumferential direction around the longitudinal axis the load resisting surfaces 6a-6d are spaced from each other.

In the example of FIG. 19, the load resisting surfaces 6a-6d of the first set form together with the load resisting surfaces 6a-6d form in the non-expanded state a cylindrical outer wall of the implant which extends from the distal end 41 towards the proximal end 40. The load resisting surfaces 6a-6d forming the cylinder are curved or bend in the circumferential direction around the axis of the cylinder. The cylinder is in this example a straight cylinder and the load resisting surfaces are unbend in the axial direction of the cylinder body they form. As can e.g. be seen in FIGS. 19A, 20A, 21A and 22, the curvature of the load resisting surfaces 6a-6d and the angle between them is such that in the non-expanded state the cylinder is in the circumferential direction almost continuous with only a very small gap between the load resisting surfaces. Upon expansion, the load resisting surfaces 6a-6d of the first set and/or the second set move radially outwards and this gap increases to almost be equal to the width of the load resisting surface in the third expanded states (that is on expansion of all sets of movable parts).

FIG. 20 shows the fourth example in a first expanded state. As can be seen most clearly in FIGS. 20B and 20D in this example the first set 3 is expanded in the first expanded state. The second set is not expanded. FIG. 21 shows the fourth example in a second expanded state. In the fourth example, the load resisting surfaces lay in the non-expanded state close to the distal end 41 but do not project beyond the distal end 41. As can e.g. be seen in FIGS. 20B and 21B, upon maximal expansion of the respective movable part, the load resisting surfaces 6a-6d project from the support structure 2 beyond the distal end. As can be seen most clearly in FIGS. 21B and 21D in this example in the second expanded state the second set 3 is expanded. The first set is not expanded.

In FIG. 22 the fourth example is shown in a third expanded state, in which both the first set and the second set are expanded. As can be seen, in this example when in the third expanded state both sets are maximally expanded, the expandable structure 5 of the implant has the shape of a fragmented cylinder, with the load resisting surfaces forming cylinder segments which in the circumferential direction of the cylinder are spaced apart from each other. In this example, the cylinder segments are parallel to each other and upon maximal expansion of all movable parts radially at the same distance from the axis of the cylinder. However, alternatively, for instance, upon expansion the cylinder segments may rotate and e.g. form a fragmented cone upon expansion. Also, for example, in some implementations some or all of the cylinder segments may be closer or further away from the axis than other cylinder segments.

In the foregoing specification, the invention has been elucidated with reference to specific examples of embodiments of the invention. It will, however, be evident that various modifications and changes may be made therein without departing from the broader scope of the invention as set forth in the appended claims, and that the examples are not intended to limit the scope of the claims.

For example, although in the example an implant for spinal surgery has been described, the implant can be implemented to be used in other bones. Furthermore, if the load resisting surfaces 6 are present this may be an anchor for other medical devices. For instance, this can be implemented as an anchor for transfixation pins to which a connecting bar may be fixated in external or internal skeletal fixation. The load resisting surface 6 can alternatively or additionally be implemented as stem for, for example, an artificial femoral, hip or shoulder joint. Likewise, the load resisting surface 6 can be implemented as an anchor for an electronic medical device or for a medical device releasing a pharmaceutically active component.

Some or all of the components of the device 1 may be made of a biocompatible material. The biocompatible material may for example be a material out of the group consisting of: metals, metal compounds, metal alloys, metal composites, polymers, ceramics and combinations of materials of this group. The biocompatible material can contain a metal out of the group consisting of: titanium, tantalum, niobium, stainless steel, cobalt chrome alloys, zirconia, or a compound, alloy or composite thereof. Other suitable biocompatible materials can contain a polymer out of the group consisting of polyaryletherketone, polyether ether ketone, polyetherketoneketone. In this respect, all components of the implant may be made of the same material, different components may be made of different materials and a component may be composed of different materials or of a single material.

In some implementations, the implant 1 may be osseoincorporable. That is, device 1 may be incorporated into the bone by ingrowth of bone matter inside the implant 1, such as in the bases 17, in addition to bone on-growth on the interfaces between device 1 and the bone, such as on the load resisting surfaces described. Device 1 may form a scaffold for bone tissue in the cavity. More specifically, the solid parts of the implant may provide a seed surface for bone material, and, after implantation, form a substrate on which osteoblasts and stem cells can grow. Without wishing to be bound to theory, it is currently believed that the solid parts initially form a seed layer for a cell growth substrate. The cell growth substrate can for example be formed by substances adsorbed to the surface of the solid parts, like proteins, water molecules and/or lipids. Also, the substrate may comprise substances attached to the solid parts of the bulk block, like blood platelets. After formation of the growth substrate, the bone may grow. For example, in case of osseo-integration, osteoblasts or their progenitors, such as osteochondro-progenitor cells or mesenchymal stem cells, will grow thereon and subsequently form the bone matrix inside, thus creating an intimate bond between the vertebra 100 and the implant 1.

One, or more than one, or all of the parts may be non-degradable in-vivo or in-situ. This allows a permanent structure. For example, the anchor body 4 may be made of a non-degradable metal containing material, whereas e.g. a movable part 3a-3d made be made of a degradable material. Alternatively or additionally, one, or more than one, or all of the parts may be bio-degradable in-vivo. This allows e.g. to place a temporary implant, or an implant with temporary parts, without requiring surgery to remove the implant. Also, for instance, the bio-degradable degradable part may fill a gap between a non-degradable part and tissue to be regrown, such as bone. This allows e.g. placing an implant at a location in a space larger than the implant, expanding the implant such that the degradable part bridges the space between the non-degradable part and the edge of the gap. The degradable part can then disappear while the gap fills, e.g. by tissue regrowth. For example, the movable parts 3a-3d may be biodegradable while the anchor body, or at least a core thereof, is made of a non-degradable material, such as a non-corrosive metal. This allows to anchor the implant 1 during the healing period and if a load resisting surface 6a-6d is present keep the load resisting surface 6a-6d anchored after the healing period even after the expandable structure 5 has decomposed. In addition, for example an outer sleeve of the anchor body may be biodegradable while the core is made of a stiff, non-degradable material (e.g. Ti or biocompatible Ti-alloys). This similarly allows to firmly anchor the implant 1 during healing while, due to the degrading of the outer sleeve, after healing the anchor body 4 be easily removed. In such a case for example the expandable structure can be left in the bone, e.g. when it has completely osseo-integrated therein.

The anchor body 4 may for example be made from materials different from the movable parts 3a-3d. This allows them to have different properties, such as a rigid anchor body 4 and a flexible movable part 3a-3d or vice-versa. One, or more than one, or all of the anchor body 4 and movable parts 3a-3d may be non-degradable in-vivo or in-situ. This allows a permanent implant, e.g. suitable for an implant which serves as an anchor for a prosthesis.

In this, for instance, the non-degradable parts to be removed after healing, may have a closed-surface to avoid integration, such as osseo-integration, in the vertebra 100 while the degradable parts have an open, porous surface to allow osseo-integration. Alternatively, the non-degradable parts may integrate into the vertebra 100 and e.g. osseo-integrate. For example, the anchor body 4 may be biodegradable. This allows to initially anchor the implant 1. When the anchor body 4 degrade and the movable parts 3a-3d integrate, e.g. by osseo-integration, the adherence between the integrated parts and the vertebra 100 can take over the anchoring function. This allows e.g. to reduce prolonged locally high pressure caused by the anchor body 4 pressing into the vertebra 100 and, without being bound to theory, is believed to reduce secondary complications post-surgery.

Furthermore, one or more of the anchor body 4, the load resisting surface 6, the movable part 3a-3d, the cap 45, the transmission system 7, the elongate shaft 10 or other elements may be a monolithic body.

Other modifications, variations and alternatives are also possible. The specifications and drawings are, accordingly, to be regarded in an illustrative rather than in a restrictive sense. In the claims, any reference signs placed between parentheses shall not be construed as limiting the claim.

The terms “front,” “back,” “top,” “shielded,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein. The term “side” is not used in a strict mathematical sense and does not need to be the face of a geometrical shape. The word ‘comprising’ does not exclude the presence of other elements or steps than those listed in a claim. Furthermore, the terms “a” or “an,” as used herein, are used in the sense of “one, or more than one” and not as being limited to one. Also, the use of introductory phrases such as “at least one” and “one, or more than one” in the claims should not be construed to imply that the introduction of another claim element by the indefinite articles “a” or “an” limits any particular claim containing such introduced claim element to inventions containing only one such element, even when the same claim includes the introductory phrases “one, or more than one” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

### List of Reference Numbers

1 expandable, intra-osseous implant
2 support structure
3,3′ set of movable parts
3a,3b,3c,3d movable parts
4 anchor body
5 expandable structure
6a,6b,6c,6d load resisting surface of movable part
7 transmission system
8,8′ first, second transmission
9 first fixed element
10 elongate shaft
11 first movable element
12 second fixed element
13 tubular element
14 second movable element
15 outer surface of support structure
16 tool interface
16a,16b drive part
17 base of movable part
18 first side of movable part
19 second side of movable part
20 first wall of movable part
21 second wall of movable part
25 first surgical driver tool
26 elongated part
27 first engaging end
28 first handle
29 second surgical driver tool
30 elongated part
31 second engaging end
32 second handle
40 proximal end
41 distal end
42 space
43 opposite openings
44 prongs
45 cap
46 hole
47 cone-shaped part
48 outer surface
48a profiled area
48b unprofiled area
49 thread
100 vertebra (bone)
101 vertebral arch
102 vertebral body
103 inter-vertebral disc
104 cannula
105 anterior wall
106 intra-osseos cavity
107 pedicle
108 lower vertebral endplate
109 upper vertebral endplate
110 threaded region
130 openings
131 fluid channel
200 surgical tool set
201 hollow tubular body
202 open ends
203 retainer
204 sleeve
205 retainer handle
206 grip
207 clamp
300,301 ring-shaped member
302 slot

1. An expandable intra-osseous implant for a bone, such as a vertebra, of a human or non-human mammal, the implant comprising:
a support structure defining a longitudinal axis;
a first set of movable parts movably mounted to the support structure and in an unexpanded state of the implant circumferentially spaced around the longitudinal axis from each other;
a second set of movable parts movably mounted to the support structure and circumferentially spaced around the longitudinal axis from each other in the unexpanded state, which second set of movable parts is offset in a longitudinal direction from the first set of movable parts;
each movable part in the first set of movable parts and the second set of movable parts being movable relative to the support structure in a radial direction away from the longitudinal axis from a first position in the unexpanded state to a second position in an expanded state of the implant, and each movable part comprising a load resisting surface for resisting a load external to the implant;
a transmission for actuating movement of each movable part of the first and second sets of movable parts along a predetermined path from the first position to the second position, wherein in the expanded state said movable parts are supported against the external load to maintain the respective movable part in its second position.

2. The implant of claim 1, wherein:
the first set of movable parts comprises a first movable part which is movable from its first position in a first radial direction and a second movable part which is movable from its first position in a second radial direction extending in a direction opposite to the first radial direction;
the second set of movable parts comprises a first movable part which is movable from its first position in a third radial direction and a second movable part which is movable from its first position in a fourth radial direction extending in a direction opposite to the third radial direction.

3-5. (canceled)

6. The implant of claim 1, wherein:
the first set of movable parts comprises a first movable part and the second set of movable parts comprises a first movable part and the first position of the first movable part of the first set is circumferentially spaced around the longitudinal axis relative to the first position of first movable part of the second set.

7. (canceled)

8. The implant of claim 1, wherein the movable parts of the first set of movable parts are movable from their first position to their second position independently from a movement of the movable parts of the second set of movable parts.

9. The implant of claim 1, wherein the transmission couples the movement of at least one movable part of the first set of movable parts to a movement of at least another movable part of the first set of movable parts.

10. The implant of claim 1, wherein the transmission couples the movement of at least one movable part of the second set of movable parts to a movement of at least another movable part of the second set of movable parts.

11-12. (canceled)

13. The implant of claim 1, wherein, in the expanded state, the load resisting surface of at least one of the movable parts of the first set of movable parts is oriented parallel to the load resisting surface of at least one movable part of the second set of movable parts.

14. The implant of claim 1, wherein, in the expanded state, the load resisting surface of at least one of the movable parts of the first set of movable parts is oriented non-parallel to the load resisting surface of at least one movable part of the second set of movable parts.

15. (canceled)

16. The implant of claim 1, wherein the first set of movable parts and the second set of movable parts both comprise at least one overhanging movable part which comprises a base and of which overhanging movable part the load resisting surface projects in the longitudinal direction from the base, and wherein the base of the overhanging movable part of the first set is located further away from a proximal end of the implant and closer to a distal end of the implant than a base of the overhanging movable parts in the second set of movable parts.

17. (canceled)

18. The implant of claim 1, further comprising:
a tool interface for a surgical tool, the tool interface engaging on the transmission to actuate the movement of the movable parts with the surgical tool.

19-20. (canceled)

21. The implant of claim 1, wherein, at least one of the movable parts is a movable bulk part, the movable bulk part comprises:
a plateau with a load facing side at which the load resisting surface is located;
a base extending in the expanded state from a side of the plateau facing away from the load facing side, in a direction opposite to the direction of expansion, the base defining the volume of the movable bulk part.

22. The implant of claim 21, wherein the base comprises a first panel extending in the direction opposite to the direction of expansion from a top-side surface and a second panel extending in the direction opposite to the direction of expansion from the top side surface, the panels being spaced apart and enclosing at respective sides the volume.

23-25. (canceled)

26. The implant of claim 1, comprising:
an expansion structure to be admitted in an intra-osseous cavity, which comprises the first set of movable parts, the second set of movable parts, and the support structure; an anchor body for anchoring the implant to a part of the bone outside the cavity, the anchor body being fixated to the expansion structure for holding the expansion structure in the intra-osseous cavity in position relative to the part of the bone outside the cavity, which anchor body comprises a distal end and a proximal end, in a longitudinal direction from the distal end towards the proximal end at a distance from the distal end;
a tool interface for a surgical tool located at the proximal end, the tool interface engaging on the transmission to actuate the movement of the movable parts with the tool and the tool interface comprising:
a coupling for the tool,
the coupling being movable relative to the proximal end of the anchor body by a force exerted on the coupling by the tool, and
the coupling engaging on the transmission to transfer at least a part of said force, when exerted on the coupling, to at least one engaged movable part out of the movable parts and thereby actuate movement of the engaged movable part along the predetermined path in the direction of expansion.

27-28. (canceled)

29. The implant of claim 1, dimensioned for percutaneous placement in a vertebra.

30-41. (canceled)

42. A method of orthopaedic surgery of a bone, such as a vertebra, of a human or non-human mammal with an expandable intra-osseous implant, the method comprising:
positioning the implant in the bone, the implant comprising:
a support structure defining a longitudinal axis;
a first set of movable parts movably mounted to the support structure and in an unexpanded state of the implant circumferentially spaced around the longitudinal axis from each other;
a second set of movable parts movably mounted to the support structure and circumferentially spaced around the longitudinal axis from each other in the unexpanded state, which second set of movable parts is offset in a longitudinal direction of the implant from the first set of movable parts;
each movable part in the first set of movable parts and the second set of movable parts being movable relative to the support structure in a radial direction away from the longitudinal axis from a first position in the unexpanded state to a second position in an expanded state of the implant, and each movable part comprising a load resisting surface for resisting a load external to the implant;
a transmission for actuating movement of each movable part of the first and second sets of movable parts along a predetermined path from the first position to the second position, wherein in the expanded state said movable parts are supported against the external load to maintain the respective movable part in its second position; and
the method further comprising expanding the implant, the expanding comprising the transmission actuating said movement for at least one movable part of at least one set of movable parts.

43. A surgical tool for expanding an expandable intra-osseous implant, the implant comprising a support structure defining a longitudinal axis;
a first set of movable parts movably mounted to the support structure and in an unexpanded state of the implant circumferentially spaced around the longitudinal axis from each other;
a second set of movable parts movably mounted to the support structure and circumferentially spaced around the longitudinal axis from each other in the unexpanded state, which second set of movable parts is offset in a longitudinal direction of the implant from the first set of movable parts;
each movable part in the first set of movable parts and the second set of movable parts being movable relative to the support structure in a radial direction away from the longitudinal axis from a first position in the unexpanded state to a second position in an expanded state of the implant, and each movable part comprising a load resisting surface for resisting a load external to the implant;
a transmission for actuating movement of each movable part of the first and second sets of movable parts along a predetermined path from the first position to the second position, wherein in the expanded state said movable parts are supported against the external load to maintain the respective movable part in its second position;
the surgical tool comprising: a driver engageable with the transmission of the implant to selectively move at least one movable part along the predetermined path.

44. The surgical tool as claimed in claim 43, comprising:
a hollow tubular body with open ends, for providing access to the outside of a bone;
the surgical tool comprising an elongated part which fits into the hollow tubular body and having a tip which when the surgical tool is in an engaged state is located at a distal end of the hollow tubular body and engages with the transmission of the implant.

45. The surgical tool of claim comprising a retainer which fits into the hollow tubular body and which has a retaining state in which the retainer engages on the implant to hold the implant in position when the surgical tool engages on the transmission and drives the movement of the movable part.

46. The surgical tool of claim 45, wherein:
the retainer has a hollow tubular inside which extends, when the retainer is positioned in the hollow tubular body, parallel to the hollow tubular body, which hollow tubular inside has at each longitudinal end an opening;
the surgical tool fits with the tip positioned at the distal end in the hollow tubular inside; and
in the engaged state the surgical tool is movable relative to the retainer to drive the movement with the tip engaged with the transmission.

47. (canceled)

48. The surgical tool of claim 44 comprising:
a first surgical driver tool for engaging with the transmission to drive movement of a movable part of a first set of movable parts;
a second surgical driver tool for engaging with the transmission to drive movement of a movable part of the second set of movable parts.

49. (canceled)
